SEQUENCE LISTING (15 sequences)

SEQ ID NO: 1 - DNA, 33 bp, synthetic construct
  source 1..33: mol_type = other DNA
tcgtcggcagcgtcagatgtgtataagagacag

SEQ ID NO: 2 - DNA, 34 bp, synthetic construct
  source 1..34: mol_type = other DNA
gtctcgtgggctcggagatgtgtataagagacag

SEQ ID NO: 3 - DNA, 19 bp, synthetic construct
  source 1..19: mol_type = other DNA
ctgtctcttatacacatct

SEQ ID NO: 4 - DNA, 14 bp, synthetic construct
  source 1..14: mol_type = other DNA
tcgtcggcagcgtc

SEQ ID NO: 5 - DNA, 15 bp, synthetic construct
  source 1..15: mol_type = other DNA
gtctcgtgggctcgg

SEQ ID NO: 6 - DNA, 19 bp, synthetic construct
  source 1..19: mol_type = other DNA
  misc_feature 1: note = This sequence may be connected with two 18-atom hexa-ethyleneglycol spacers to SEQ ID NO: 8
  misc_feature 1: note = This sequence may be connected with two 18-atom hexa-ethyleneglycol spacers to SEQ ID NO: 12
agatgtgtataagagacag

SEQ ID NO: 7 - DNA, 15 bp, synthetic construct
  source 1..15: mol_type = other DNA
tcactcaagaacagc

SEQ ID NO: 8 - DNA, 29 bp, synthetic construct
  source 1..29: mol_type = other DNA
  misc_feature 29: note = This sequence may be connected with two 18-atom hexa-ethyleneglycol spacers to SEQ ID NO: 6
tcgtcggcagcgtctcactcaagaacagc

SEQ ID NO: 9 - skipped

SEQ ID NO: 10 - DNA, 30 bp, synthetic construct
  source 1..30: mol_type = other DNA
gctgttcttgagtgaccgagcccacgagac

SEQ ID NO: 11 - DNA, 29 bp, synthetic construct
  source 1..29: mol_type = other DNA
gctgttcttgagtgagacgctgccgacga

SEQ ID NO: 12 - DNA, 30 bp, synthetic construct
  source 1..30: mol_type = other DNA
  misc_feature 30: note = This sequence may be connected with two 18-atom hexa-ethyleneglycol spacers to SEQ ID NO: 6
gtctcgtgggctcggtcactcaagaacagc

SEQ ID NO: 13 - skipped

SEQ ID NO: 14 - DNA, 28 bp, synthetic construct
  source 1..28: mol_type = other DNA
aatgatacggcgaccaccgagactacac

SEQ ID NO: 15 - DNA, 24 bp, synthetic construct
  misc_difference 22: note = g could be guanine or a modified guanine, e.g., an 8-oxo-guanine
  source 1..24: mol_type = other DNA
caagcagaagacggcatacgagat

(19) United States
(12) Patent Application Publication
(10) Pub. No.: US 2024/0026348 A1
(43) Pub. Date: Jan. 25, 2024

(54) Methods of Preparing Directional Tagmentation Sequencing Libraries Using Transposon-Based Technology with Unique Molecular Identifiers for Error Correction

(71) Applicants: Illumina, Inc., San Diego, CA (US); Illumina Cambridge Limited, Cambridge (GB)

(72) Inventors: Susan C. Verity, Cambridge (GB); Robert Scott Kuersten, Madison, WI (US); Niall Anthony Gormley, Cambridge (GB); Andrew B. Kennedy, San Diego, CA (US); Sarah E. Shultzaberger, San Diego, CA (US); Andrew Slatter, Cambridge (GB); Emma Bell, Cambridge (GB); Sebastien Georg Gabriel Ricoult, Nr Saffron Walden (GB); Grace DeSantis, San Diego, CA (US); Fiona Kaper, San Diego, CA (US); Han-Yu Chuang, San Diego, CA (US); Oliver Jon Miller, Nr Saffron Walden (GB); Jason Richard Betley, Buntingford (GB); Stephen M. Gross, San Diego, CA (US); Mats Ekstrand, San Diego, CA (US)

(73) Assignees: Illumina, Inc., San Diego, CA (US); Illumina Cambridge Limited, Cambridge (GB)

(21) Appl. No.: 18/476,719

(22) Filed: Sep. 28, 2023

Related U.S. Application Data
(63) Continuation of application No. PCT/US22/22379, filed on Mar. 29, 2022.
(60) Provisional application No. 63/168,802, filed on Mar. 31, 2021.

(51) Int. Cl. C12N 15/10

(52) U.S. Cl. CPC C12N 15/1065; C12N 15/1068

(57) ABSTRACT

Materials and methods for preparing nucleic acid libraries for next-generation sequencing are described herein. A variety of approaches are described relating to the use of unique molecular identifiers with transposon-based technology in the preparation of sequencing libraries. Also described herein are sequencing materials and methods for identifying and correcting amplification and sequencing errors.

### CROSS-REFERENCE TO RELATED APPLICATION

This application is a bypass continuation of PCT/US2022/022379, filed Mar. 29, 2022, which claims the benefit of priority of U.S. Provisional Application No. 63/168,802, filed Mar. 31, 2021, which is incorporated by reference herein in its entirety for any purpose.

### SEQUENCE LISTING

This application is filed with a Sequence Listing in electronic format. The Sequence Listing is provided as a file entitled “2023-09-28_01243-0024-00US_ST26” created on Sep. 26, 2023, which is 15,273 bytes in size. The information in the electronic format of the sequence listing is incorporated herein by reference in its entirety.

### DESCRIPTION

### Field

This application relates to preparation of DNA and RNA sequencing libraries using transposon-based technology to incorporate unique molecular identifiers (UMIs) that increase sequencing sensitivity of low frequency variants.

### BACKGROUND

Next-generation sequencing (NGS) has enabled cancer researchers to assess numerous genes in single assay using highly accurate sequencing data. However, any synthesis-based method involves inherent errors. Although the error rate is low enough (less than 0.5%) to successfully accomplish many NGS-based applications, new approaches that use noninvasive or other methods for sample collection that result in a lower concentration of target nucleic acid may require a lower error rate. For example, analysis of cell free DNA (cfDNA) can be used to detect somatic variants in blood without the need for biopsy; however, the low percentage of circulating tumor DNA (ctDNA) within total cfDNA causes variant allele frequencies to exist near the limit of detection of existing methods. Artifacts that may arise from library preparation methods can be mistaken as low frequency variants, thereby decreasing the sensitivity and reliability of the methods.

Transposon-based technologies can be used to prepare whole-genome sequencing libraries. For example, the Illumina DNA Prep (RUO), previously known as Nextera DNA Flex Library Prep, supports a broad nucleic acid input range (1-500 ng), multiple sample types, and both small and large genomes. In under 4 hours, a library of 350-base pair fragments can be generated and, by treating the target nucleic acids with transposome complexes so that the nucleic acids are simultaneously fragmented and tagged (“tagmented”) for sequencing.

The libraries prepared according to transposon-based technologies may be improved by incorporation of Unique Molecular Identifiers (UMIs) to lower the rate of inherent errors in NGS data. Integration of UMIs into a sequencing library enables the UMI Error Correction App to recognize multiple reads from the same target molecule and collapse them into a single read, reducing errors in final variant calls. UMIs in combination with stranded (i.e., forked) libraries can resolve individual strand molecules in sequencing data. The present disclosure provides materials and methods for preparing UMI libraries using transposon-based technologies.

### SUMMARY

The present disclosure relates to materials, compositions, and methods for preparing nucleic acid sequencing libraries comprising UMIs using transposon-based technology.

Embodiment 1 is a method of producing a double-stranded nucleic acid library wherein each fragment in the library comprises a unique molecular identifier (UMI) wherein the method comprises: (a) applying a sample comprising double-stranded target nucleic acids to a first transposome complex comprising: (i) a first transposase, (ii) a first transposon comprising a first 3′ end transposon end sequence, a first adapter sequence, and a first UMI, and (iii) a second transposon comprising a sequence all or partially complementary to the first 3′ end transposon end sequence; (b) tagmenting the double-stranded target nucleic acids with the first transposome complex to produce tagmented double-stranded target nucleic acid fragments, wherein each tagmented double-stranded target nucleic acid fragment comprises the first adapter sequence and the first UMI, (c) releasing the tagmented double-stranded target nucleic acid fragments from the first transposome complex, (d) optionally extending the tagmented double-stranded target nucleic acid fragments, (e) optionally ligating the first transposon with the tagmented double-stranded target nucleic acid fragments or with the extended, tagmented double-stranded target nucleic acid fragments, (f) producing tagmented double-stranded target nucleic acid fragments, and (g) amplifying the tagmented double-stranded target nucleic acid fragments.

Embodiment 2 is the method of embodiment 1, wherein the first UMI in the first transposon is located between the first adapter sequence and the first 3′ transposon end sequence.

Embodiment 3 is the method of embodiment 1 or 2, wherein the first adapter sequence in the first transposon is located between the first UMI and the first 3′ transposon end sequence.

Embodiment 4 is the method of any one of embodiments 1-3, further comprising a second transposome complex comprising: (a) a second transposase, (b) a third transposon comprising a second adapter sequence and a second 3′ transposon end sequence, and (c) a fourth transposon comprising a sequence all or partially complementary to the second 3′ end transposon end sequence.

Embodiment 5 is the method of embodiment 4, wherein the tagmenting step produces tagmented double-stranded target nucleic acid fragments comprising: (a) a first strand comprising the first adapter sequence and the first UMI, and (b) a second strand comprising the second adapter sequence.

Embodiment 6 is the method of embodiment 4 or 5, wherein (a) the third transposon further comprises a second UMI, and (b) the second adapter sequence is located between the second UMI and the second 3′ transposon end sequence.

Embodiment 7 is the method of embodiment 6, wherein the tagmenting step produces double-stranded target nucleic acid fragments comprising: (a) a first strand comprising the first adapter sequence and the first UMI, and (b) a second strand comprising the second adapter sequence and the second UMI.

Embodiment 8 is a method of producing a double-stranded nucleic acid library wherein each fragment in the library comprises a UMI wherein the method comprises: (a) applying a sample comprising double-stranded target nucleic acids to a transposome complex comprising: (i) a transposase, (ii) a first transposon comprising a first 3′ end transposon end sequence and a first adapter sequence, and (iii) a second transposon comprising a sequence all or partially complementary to the first 3′ end transposon end sequence; (b) tagmenting a first strand of the double-stranded target nucleic acids with the transposome complex to produce tagmented double-stranded target nucleic acid fragments, wherein each tagmented double-stranded target nucleic acid fragment comprises the first adapter sequence, (c) releasing the tagmented double-stranded target nucleic acid fragments from the transposome complex, (d) hybridizing a polynucleotide comprising a second adapter sequence, a UMI, and a sequence all or partially complementary to the first 3′ end transposon sequence, (e) optionally extending a second strand of the tagmented double-stranded target nucleic acid fragments, (f) optionally ligating the polynucleotide with the tagmented double-stranded target nucleic acid fragments or with the extended tagmented double-stranded target nucleic acid fragments, (g) producing tagmented double-stranded target nucleic acid fragments comprising the UMI, wherein the UMI is located directly adjacent to the 3′ end of an insert DNA, and (h) amplifying the tagmented double-stranded target nucleic acid fragments comprising the UMI.

Embodiment 9 is a method of producing a double-stranded nucleic acid library wherein each fragment in the library comprises a UMI wherein the method comprises: (a) applying a sample comprising double-stranded target nucleic acids to a transposome complex comprising: (i) a transposase, (ii) a first transposon comprising a first 3′ end transposon end sequence and a first adapter sequence, and (iii) a second transposon comprising a sequence all or partially complementary to the first 3′ end transposon end sequence; (b) tagmenting a first strand of the double-stranded target nucleic acids with the transposome complex to produce tagmented double-stranded target nucleic acid fragments, wherein each tagmented double-stranded target nucleic acid fragment comprises the first adapter sequence, (c) releasing the tagmented double stranded target nucleic acid fragments from transposome complex, (d) hybridizing a first polynucleotide comprising a UMI, and a second adapter sequence, (e) optionally adding a second polynucleotide comprising regions complementary to the first polynucleotide to produce a double-stranded adapter, (f) optionally extending a second strand of the tagmented double-stranded target nucleic acid fragments, (g) optionally ligating the second polynucleotide with the second strand of the extended tagmented double-stranded target nucleic acid fragments, (h) producing tagmented double stranded target nucleic acid fragments comprising the UMI, wherein the UMI is located between the double-stranded target nucleic acid fragments and the second adapter sequence, and (i) amplifying the tagmented double-stranded target nucleic acid fragments comprising the UMI.

Embodiment 10 is the method of embodiment 9, wherein after the hybridizing step, the method further comprises (a) extending a second strand of the double-stranded target nucleic acid fragments, and (b) copying the first polynucleotide.

Embodiment 11 a method of producing a double-stranded nucleic acid library wherein each fragment in the library comprises two different UMIs wherein the method comprises (a) applying a sample comprising double-stranded target nucleic acids to: (i) a first transposome complex comprising: (1) a first transposase and (2) a first forked adapter comprising (a) a first transposon on a first strand of the double-stranded target nucleic acid fragments, and (b) a second transposon, wherein the first transposon comprises a first 3′ end transposon end sequence, a first copy of a first adapter sequence, and a first UMI, and the second transposon comprises a first copy of a second adapter sequence, and a sequence all or partially complementary to the first 3′ end transposon end sequence and the first UMI; further wherein the first copy of the first adapter sequence is single-stranded and the first copy of the second adapter sequence includes a double-stranded portion; and (ii) a second transposome complex comprising: (1) a second transposase and (2) a second forked adapter comprising (a) a third transposon on a second strand of the double-stranded target nucleic acid fragments, and (b) a fourth transposon, wherein the third transposon comprises a second 3′ end transposon end sequence, a second copy of the first adapter sequence, and a second UMI, and the third transposon comprises a second copy of the second adapter sequence, and a sequence all or partially complementary to the second 3′ end transposon end sequence and the second UMI; further wherein the second copy of the first adapter sequence is single-stranded and the second copy of the second adapter sequence includes a double-stranded portion; (b) tagmenting the double-stranded target nucleic acids with the forked adapters to produce tagmented double-stranded target nucleic acid fragments, wherein each tagmented double-stranded target nucleic acid fragment comprises the first and second copies of the first adapter sequence, the first UMI, the first and second copies of the second adapter sequence, and the second UMI, (c) releasing the tagmented double-stranded target nucleic acid fragments from the transposome complexes, (d) optionally extending the tagmented double-stranded target nucleic acid fragments, (e) ligating the second and fourth transposons with the double-stranded target nucleic acid fragments or with the extended tagmented double-stranded target nucleic acid fragments, (f) producing tagmented double-stranded target nucleic acid fragments, and (g) amplifying the tagmented double-stranded target nucleic acid fragments.

Embodiment 12 is a method of producing a double-stranded nucleic acid library wherein each fragment in the library comprises four different UMIs wherein the method comprises (a) applying a sample comprising double-stranded target nucleic acids to: (i) a first transposome complex comprising: (1) a first transposase and (2) a first forked adapter comprising (a) a first transposon on a first strand of the double-stranded target nucleic acid fragments, and (b) a second transposon, wherein the first transposon comprises a first 3′ end transposon end sequence, a first copy of a first adapter sequence, a first copy of a first UMI, and a first copy of a second adapter sequence, and the second transposon comprises a sequence all or partially complementary to the first 3′ end transposon end sequence, a first copy of a third adapter sequence, a first copy of a second UMI, and a fourth adapter sequence; further wherein the first copies of the first, second, and third adapter sequences are single-stranded and the fourth adapter sequence includes a double-stranded portion; and (i) a second transposome complex comprising: (1) a second transposase and (2) a second forked adapter comprising (a) a third transposon on a second strand of the double-stranded target nucleic acid fragments, and (b) a fourth transposon, wherein the third transposon comprises a second 3′ end transposon end sequence, a first copy of a fifth adapter sequence, a first copy of a third UMI, and a first copy of a sixth adapter sequence; the fourth transposon comprises a sequence all or partially complementary to the second 3′ end transposon end sequence, a first copy of a seventh adapter sequence, a first copy of a fourth UMI, and an eighth adapter sequence; further wherein the first copies of the fifth, sixth, and seventh adapter sequences are single-stranded and the eighth adapter sequence includes a double-stranded portion; (b) tagmenting the double-stranded target nucleic acids with the forked adapters to produce tagmented double-stranded target nucleic acid fragments, wherein each tagmented double-stranded target nucleic acid fragment comprises the first copies of the first, second, third, fifth, sixth, and seventh adapter sequences; the first copies of the first, second, third, and fourth UMIs; the sixth adapter sequence; and the eighth adapter sequence, (c) releasing the tagmented double-stranded target nucleic acid fragments from the transposome complexes, (d) optionally extending the tagmented double-stranded target nucleic acid fragments, (e) ligating the second and fourth transposons with the double-stranded target nucleic acid fragments or with the extended tagmented double-stranded target nucleic acid fragments, (f) producing tagmented double-stranded target nucleic acid fragments, and (g) amplifying the tagmented double-stranded target nucleic acid fragments.

Embodiment 13 is the method of any one of embodiments 6, 7, 11 or 12, wherein the first, second, third, and fourth UMIs may be complementary or different sequences.

Embodiment 14 is the method of any one of embodiments 1-13, wherein the double-stranded target nucleic acids are double-stranded DNA.

Embodiment 15 is the method of any one of embodiments 1-13, wherein the double-stranded target nucleic acids are ctDNA.

1 Embodiment 16 is the method of any one of embodiments 1-13, wherein the double-stranded target nucleic acids are cfDNA.

Embodiment 17 is the method of any one of embodiments 1-13, wherein the double-stranded target nucleic acids are RNA.

Embodiment 18 is the method of any one of embodiments 1-13, wherein double-stranded target nucleic acids are cDNA or DNA:RNA duplexes are generated from RNA.

Embodiment 19 is the method of any one of embodiments 1-18, wherein the first adapter sequence is a 5′ first-read sequencing adapter sequence.

Embodiment 20 is the method of any one of embodiments 1-19, wherein the second adapter sequence is a 5′ second-read sequencing adapter sequence.

Embodiment 21 is the method of any one of embodiments 1-20, wherein the first and second adapter sequences are 5′ first-read and 5′ second-read sequencing adapter sequences.

Embodiment 22 is the method of any one of embodiments 1-21, wherein the 5′ first-read and 5′ second-read sequencing adapter sequences comprise unique primer binding sites.

Embodiment 23 is the method of any one of embodiments 1, 2, 4-8, or 13-22, wherein the first UMI is on the first strand of the tagmented double-stranded target nucleic acid fragments.

Embodiment 24 is the method of any one of embodiments 1, 3, 5-7, 13-22, wherein a first copy of the first UMI is on the first strand and a second copy of the first UMI is on the second strand of the tagmented double-stranded target nucleic acid fragments.

Embodiment 25 is the method of any one of embodiments 1-7, 13-22, wherein the first UMI is on the first strand of the tagmented double-stranded target nucleic acid fragments, the second UMI is on the second strand of the tagmented double-stranded target nucleic acid fragments.

Embodiment 26 is the method of any one of embodiments 1-25, wherein the first, second, third, or fourth transposon further comprises a biotin tag.

Embodiment 27 is the method of any one of embodiments 1-26, wherein the first, second, third, or fourth transposon further comprises a first unique primer binding sequence.

Embodiment 28 is the method of embodiment 27, wherein the first, second, third, or fourth transposon further comprises a second unique primer binding sequence.

Embodiment 29 is the method of embodiment 27 or 28, wherein the unique primer binding sequence comprises A2, A14, and/or B15.

Embodiment 30 is the method of any one of embodiments 8-10 or 14-22, wherein the hybridizing step generates a forked adapter.

Embodiment 31 is the method of any one of embodiments 1-30, further comprising extending from a 3′ end of the double-stranded target nucleic acid fragments to a 5′ end of the transposons.

Embodiment 32 is the method of any one of embodiments 1-7 or 11-31, wherein the ligating step comprises ligating a 3′ end of the tagmented double-stranded target nucleic acid fragments or a 3′ end of the extended tagmented double-stranded target nucleic acid fragments with a 5′ end of the first, second, or fourth transposon.

Embodiment 33 is the method of any one of embodiments 1-32, wherein the extension and/or ligating step is optionally performed in an extension ligation mix.

Embodiment 34 is the method of any one of embodiments 8, 15-22, 26-33, wherein the polynucleotide comprises a 3′ adapter comprising: (a) a hairpin UMI, (b) a hairpin UMI and a universal hybridizing tail, (c) a splint ligation adapter, or (d) a 3′ template switch oligonucleotide.

Embodiment 35 is the method of embodiment 34, wherein the hairpin UMI is stable during the extending step and/or the ligating step, but not during the amplifying step.

Embodiment 36 is the method of embodiment 34 or 35, wherein the hairpin UMI comprises a 3 or 4 base pair stem.

Embodiment 37 is the method of any one of embodiments 34-36, wherein the universal hybridizing tail comprises nucleotides that can bind to any DNA nucleotide.

Embodiment 38 is the method of any one of the embodiments 34-37, wherein the ligating step comprises ligating a 3′ end of the second strand of the tagmented double-stranded target nucleic acid fragments with a 5′ end of the universal hybridization tail.

Embodiment 39 is the method of embodiment 34, wherein (a) the polynucleotide comprises a 3′ adapter comprising a hairpin UMI, and (b) the extending step comprises extending from a 3′ end of the second strand of the tagmented double-stranded target nucleic acid fragments to a 5′ end of the hairpin UMI.

1 Embodiment 40 is the method of embodiment 39, wherein the ligating step comprises ligating the 3′ end of second strand of the extended tagmented double-stranded target nucleic acid fragments with the 5′ end of the hairpin UMI.

Embodiment 41 is the method of embodiment 34, wherein (a) the polynucleotide comprises a splint ligation adapter, and (b) the extending step comprises extending from a 3′ end of the second strand of the tagmented double-stranded target nucleic acid fragments to a 5′ end of the splint ligation adapter.

Embodiment 42 is the method of embodiment 41, wherein the extending step comprises extending 9 bases.

Embodiment 43 is the method of embodiment 41 or 42, wherein the ligating step comprises ligating the 3′ end of the second strand of the extended tagmented double-stranded target nucleic acid fragments with a 5′ end of a first strand of the splint ligation adapter.

Embodiment 44 is the method of any one of embodiments 34, wherein (a) the polynucleotide comprises a template switch oligonucleotide, and (b) the extending step comprises extending from a 3′ end of the second strand of the tagmented double-stranded target nucleic acid fragments to a junction in the template switch oligonucleotide by copying the first strand of the tagmented double-stranded target nucleic acid fragments, (c) switching templates from the first strand to an unpaired region of the 3′ template switch oligonucleotide, and (d) copying the unpaired region of the 3′ template switch oligonucleotide from the junction to a 5′ end of the unpaired region of the 3′ template switch oligonucleotide.

Embodiment 45 is the method of embodiment 44, wherein the extending, switching, and copying are performed by a polymerase capable of DNA-directed template-switching.

Embodiment 46 is the method of embodiment 44 or 45, wherein the polymerase capable of DNA-directed template-switching comprises MMLV reverse transcriptase.

Embodiment 47 is the method of any one of the embodiments 1-33, wherein the ligating step comprises ligating a 3′ end of the tagmented double-stranded target nucleic acid fragments with a 5′ end of first, second, or fourth transposon.

Embodiment 48 is the method of any one of embodiments 1-33 or 47, further comprising selecting for amplified nucleic acid fragments within a size range after the amplifying step.

Embodiment 49 is the method of any one of embodiments 1-48, wherein the amplifying step comprises adding oligonucleotides to one or both ends of the tagmented double-stranded target nucleic acid fragments for attaching the library to a solid support.

Embodiment 50 is the method of any one of embodiments 1-49, wherein the amplifying step comprises adding at least a first-read sequencing oligonucleotide and/or a second-read sequencing oligonucleotide.

Embodiment 51 is the method of any one of embodiments 1-50, wherein the amplifying step comprises adding at least a P5 oligonucleotide and a P7 oligonucleotide.

Embodiment 52 is the method of any one of embodiments 1-51, wherein the amplifying step comprises adding at least a plurality of i5 oligonucleotides and a plurality of i7 oligonucleotides.

Embodiment 53 is the method of any one of embodiments 1-52 wherein the transposome complex, the first transposome complex and/or the second transposome complex are on a solid support.

Embodiment 54 is the method of any one of embodiments 1-53, wherein the transposome complex, the first transposome complex and/or the second transposome complex are in solution.

Embodiment 55 is a method of sequencing a double-stranded nucleic acid library produced by the method of any one of embodiments 1-54, wherein the UMIs are sequenced to provide increased sensitivity in DNA sequencing.

Embodiment 56 is the method of embodiment 55, comprising binding sequencing primers having similar melting temperatures.

Embodiment 57 is the method of embodiment 55 or 56, comprising binding sequencing primers comprising a sequence all or partially complementary to unique primer binding sequences.

Embodiment 58 is the method of any one of embodiments 55-57, comprising sequencing primers with at least an A2 sequence.

Embodiment 59 is the method of any one of embodiments 55-57, comprising sequencing primers with at least an A14 sequence and a B15 sequence.

Embodiment 60 is the method of any one of embodiments 55-59, comprising sequencing primers with at least a bridged primer.

Embodiment 61 is the method of any one of embodiments 55-60, further comprising dark cycles wherein data is not being recorded for a portion of the sequencing method.

Embodiment 62 is the method of any one of embodiments 55-60, wherein the data not being recorded is sequence data associated with the 3′ transposon end sequence.

Embodiment 63 is the method of any one of embodiments 55-60, wherein the method obviates the need for dark cycles.

Embodiment 64 is the method of embodiment 1 or 9, wherein the extension step comprises a polymerase to copy the UMI or the first UMI to produce a duplex UMI.

Embodiment 65 is a transposome complex comprising: (a) a transposase, (b) a first transposon comprising a 3′ transposon end sequence and a 5′ adapter sequence, and (c) a second transposon comprising a sequence all or partially complementary to the first 3′ end transposon end sequence.

Embodiment 66 is the transposome complex of embodiment 65, wherein the 5′ adapter sequence of the first transposon comprises an A14 sequence (SEQ ID NO: 4), an A2 sequence (SEQ ID NO: 7), and/or a B15 sequence (SEQ ID NO: 5).

Embodiment 67 is the transposome complex of embodiment 65 or 66, wherein the first transposon further comprises a UMI sequence.

Embodiment 68 is the transposome complex of any one of embodiments 65-67 wherein the first or second transposon comprises A14-ME (SEQ ID NO: 1).

Embodiment 69 is the transposome complex of any one of embodiments 65-67 wherein the first or second transposon comprises B15-ME (SEQ ID NO: 2).

Embodiment 70 is the transposome complex of any one of embodiments 65-67 wherein the 3′ transposon end sequence of the first transposon comprises ME (SEQ ID NO: 6) or ME′ (SEQ ID NO: 3).

Embodiment 71 is the transposome complex of any one of embodiments 65-67 wherein the 3′ transposon end sequence of the second transposon comprises ME (SEQ ID NO: 6) or ME′ (SEQ ID NO: 3).

Embodiment 72 is the transposome complex of embodiment 67, wherein the second transposon further comprises a 3′ adapter sequence, wherein the 3′ adapter sequence of the second transposon is either partially or completely complementary to the 5′ adapter sequence of the first transposon.

1 Embodiment 73 is the transposome complex of embodiment 67, wherein the second transposon further comprises a 3′ adapter sequence, wherein no portion of the 3′ adapter sequence of the second transposon is complementary to the 5′ adapter sequence of the first transposon.

Embodiment 74 is the transposome complex of embodiment 72 or 73, wherein the 3′ adapter sequence of the second transposon comprises an A14 sequence (SEQ ID NO: 4), an A2 sequence (SEQ ID NO: 7), a B15 sequence (SEQ ID NO: 5), an X sequence, a Y′ sequence, an A sequence, and/or a B sequence.

Embodiment 75 is the transposome complex of embodiment 72 or 74, wherein the second transposon further comprises a sequence that is complementary to the UMI sequence of the first transposon.

Embodiment 76 is the transposome complex of embodiment 73 or 74, wherein the second transposon further comprises a UMI, wherein the UMI of the second transposon comprises a different sequence from the UMI of the first transposon.

Embodiment 77 is the transposome complex of embodiment 75 or 76, further comprising an oligonucleotide complementary to the B15 sequence or A14 sequence.

Embodiment 78 is the transposome complex of embodiment 76, further comprising: (a) an A adapter sequence adjacent to the A14 sequence, (b) a B adapter sequence adjacent to the B15 sequence, (c) a X adapter sequence adjacent to the ME sequence, and/or (d) a Y′ adapter sequence adjacent to the ME′ sequence.

Embodiment 79 is the transposome complex of any one of embodiments 65-78, wherein the transposome complex is immobilized to a solid support via the first or second transposon.

Embodiment 80 is the transposome complex of embodiment 77, wherein the transposome complex is immobilized to a solid support via the complementary oligonucleotide.

Embodiment 81 is the transposome complex of embodiment 79 or 80, wherein the solid support is a bead.

Embodiment 82 is a kit comprising the transposome complex of any one of embodiments 65-81.

Embodiment 83 is a kit for generating the transposome complex of any one of embodiments 65-81.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows an embodiment wherein capture oligonucleotides are used for tagmenting DNA fragments using bead-linked transposomes (BLTs).

FIG. 2 shows incorporation of unique molecular identifiers (UMIs) using A2 adapters. The method combines BLTs with a Hyb2Y workflow to produce a tagmented DNA library suitable for sequencing with the benefit of duplex UMI error correction. The UMIs may comprise randomized sequences.

FIGS. 3A-E show sequencing of duplex UMI DNA libraries prepared as described in Example 1. FIG. 3A shows standard sequencing for Illumina DNA Prep and Illumina DNA Prep with Enrichment with primers Standard Read 1, Standard Read 2, Standard i5, and Standard i7. FIG. 3B shows a Nextera sequencing method comprising 4 custom primers and 19 dark cycles. Grey arrows indicate where the custom primers anneal. FIG. 3C show the quality of every cycle in an exemplary sequencing run represented as a percent likelihood of being equal or greater than Q30. FIG. 3D shows sequencing signal intensity using i7 and i5 primers for an exemplary sequencing run. FIG. 3E compares the percent duplex families for the BLT duplex UMI design (described in FIG. 2) with the TruSight UMI (TruSight Duplex) method.

FIG. 4 shows sequencing of a duplex UMI DNA library with bridged primer rehybridization.

FIGS. 5A and 5B show the transposome structure (FIG. 5A) and workflow (FIG. 5B) for a UMI-BLT. TsTn5=transposase.

FIGS. 6A and 6B show sequencing of a duplex UMI library with dark cycles (FIG. 6A) and without dark cycles (FIG. 6B).

FIG. 7 shows % Q30 score for sequencing runs using the following methods: IDPE, TruSeq™, non-forked UMI-BLT with dark cycles, and non-forked UMI-BLT with bridged primer rehybridization. % Q30 scores are shown for Read 1 and Read 2.

FIG. 8 shows the BLT and enrichment workflows used for preparation of a DNA library with single UMIs from cfDNA. In some embodiments, a circulating nucleic acid kit (Qiagen; catalog #: 55114) was used to extract cfDNA.

FIG. 9 shows incorporation of single UMIs using classic Nextera adapters. While this method does not allow for sample indexing, standard sequencing methods can capture the incorporated UMIs from the index read. In some embodiments, standard sequencing primers are used to read the UMIs.

FIG. 10 shows % total reads which indicate that the UMIs were successfully incorporated into tagmented DNA fragments and were evenly distributed across the tagmented library.

FIGS. 11A and 11B show that a single UMI-BLT library have greater mean target coverage and higher conversion of cfDNA to library than a TruSeq™ library (shown as “No UMI” in FIG. 11A). FIG. 11A shows deduped mean target coverage as provided by Read Collapsing analysis. FIG. 11B compares the TruSeq™ method and the Single UMI-BLT method (shown as “eBBN” in FIG. 11B).

FIG. 12 shows incorporation of duplex UMIs using forked adapter capture oligonucleotides in BLTs to produce a DNA library for sequencing that is compatible with unique dual indexes (UDIs).

FIG. 13 shows incorporation of duplex UMIs using forked adapter capture oligonucleotides in BLTs to produce a DNA library for sequencing that is compatible with UDIs.

FIG. 14 illustrates Hyb2Y and ligation with a 3′ adapter containing a hairpin-UMI and a universal hybridization 5′ tail (universal hybridizing tail). This method utilizes an A14-only Tn5. A ligation step takes place after Hyb2Y; an extension step is not needed. In some embodiments, the universal hybridizing tail comprises inosine bases capable of universal Watson-Crick base-pairing. In some embodiments, the universal hybridizing tail may hybridize to A14 and/or B15. * marks the ligation junction. In some embodiments, the universal hybridization 5′ may hybridize to A14 and B15.

FIG. 15 illustrates Hyb2Y, extension, and ligation with a 3′ adapter containing a hairpin UMI. After Hyb2Y, an extension step takes place, followed by a ligation step. In some embodiments, the hairpin stem comprises 3-4 base pairs for stability. In some embodiments, there the hairpin loop comprises about 4 bases. * marks the ligation junction.

FIG. 16 illustrates Hyb2Y, extension, and ligation with a 3′ adapter complex. This method utilizes an A14-only Tn5. In some embodiments, the splint ligation adapter comprises two portions: a splint portion and a tail portion. Each portion is about 50 nucleotides long. In some embodiments, A14′, ME, and/or X may be truncated or eliminated. * marks the ligation junction.

FIG. 17 illustrates the template switch off ME-sequence method which utilizes an A14-only Tn5. A template switch extension step takes place after the hybridization step. In some embodiments, a long template switch of about 70 nucleotides may be used. In some embodiments, the switch oligonucleotide may form secondary structure on itself (i.e., fold), which precludes it from functioning as intended in an embodiment. Switch oligonucleotide folding may be circumvented by using a TruSeq™ adapter sequence in place of ME for the P7 side (indicated with ***). In some embodiments, A14′ may be truncated or omitted. ** marks the template switch junction.

FIGS. 18A-D show addition of a 3′ UMI and adapter sequence using a polymerase template switch. Tagmentation of target DNA carried out with an A14 transposome (FIG. 18A). Hyb2Y is used to add a single-stranded polymerase template switch adapter (FIG. 18B). Insert DNA is extended using a polymerase capable of switching templates from the insert DNA to the polymerase template switch adapter (FIG. 18C). PCR is used to amplify the library from A14 and B15 using sample indexes and flow cell primers (FIG. 18D).

FIGS. 19A-D show addition of a 3′ UMI using a 5′ adapter sequence and polymerase extension and proximity. Tagmentation of target DNA carried out with an A14 transposome (FIG. 19A). Hyb2Y is used to add a 5′ double-stranded adapter (FIG. 19B). Polymerase extension and proximity 5′ ligation are used to add the UMI to the insert DNA (FIG. 19C). PCR is used to amplify the library from A14 and B15 using sample indexes and flow cell primers (FIG. 19D).

FIG. 20 compares certain embodiments of adding a 3′ UMI that is in-line with, i.e., adjacent to, the insert DNA. In certain embodiments, template switch extension is used. In certain embodiments, extension and ligation is used.

FIGS. 21A-C show certain embodiments of attaching transposome complex oligonucleotides to solid support surfaces. These embodiments provide options to help with utility of BLTs with target enrichment methods that may become compromised by the presence of 5′ biotinylated library fragments. FIG. 21A shows indirect 3′ biotin attachment of Tsm adapter though complementary base pairing in the adapter. FIG. 21B shows direct 3′ biotinylation attachment. FIG. 21C shows direct 5′ biotinylation attachment.

### DESCRIPTION OF THE SEQUENCES

Table 1 provides a listing of certain sequences referenced herein. All sequences are written either N-terminus to C-terminus or 5′ to 3′, for protein and nucleic acid sequences, respectively. Certain sequences in Table 1 represent an exemplary sequence from a library of sequences. For example, as discussed in Section II.A below, “UMI” represents a library of UMI sequences. In another example, an ME sequence may contain sequence variations when compared to the exemplary ME of SEQ ID NO: 6. In the same way, an A14-ME sequence may contain sequence variations when compared to the exemplary A14-ME of SEQ ID NO: 1. Sequence variations may include, for example, nucleic acid mutations, nucleic acid substitutions, nucleic acid deletions, nucleic acid additions, nucleic acid insertions, sequence truncations, longer sequences, shorter sequences, UMI sequences, primer sequences, index tag sequences, capture sequences, barcode sequences, cleavage sequences, anchor sequences, universal sequences, spacer sequences, transposon end sequences, sequencing-related sequences, and any combination thereof. In another example, primers and adapters that relate to sequencing may refer to libraries of primers and adapters. Libraries of i5 and i7 sequences are provided by the Illumina Adapter Sequences Document #1000000002694 v15, and is hereby incorporated by reference in its entirety. In exemplary custom primers such as SEQ ID NOS: 10 and 11, the i5 and i7 portions may contain sequence variations as provided by Illumina Adapter Sequences Document #1000000002694 v15.

TABLE 1
Description the Sequences

| | | SEQ ID |
|---|---|---|
| Description | Sequences | NO |
| Exemplary A14-ME | TCGTCGGCAGCGTCAGATGTGTATAAGAGACAG | 1 |
| Exemplary B15-ME | GTCTCGTGGGCTCGGAGATGTGTATAAGAGACAG | 2 |
| Exemplary ME′ | phos-CTGTCTCTTATACACATCT | 3 |
| Exemplary A14 | TCGTCGGCAGCGTC | 4 |
| Exemplary B15 | GTCTCGTGGGCTCGG | 5 |
| Exemplary ME | AGATGTGTATAAGAGACAG | 6 |
| Exemplary A2 | TCACTCAAGAACAGC | 7 |
| Exemplary A14-A2 | TCGTCGGCAGCGTCTCACTCAAGAACAGC | 8 |
| Custom UMI 1 Read | | |
| Exemplary A14-A2- | TCGTCGGCAGCGTCTCACTCAAGAACAGC/iSp18//iSp18/ | 9 |
| spacer-ME Custom | AGATGTGTATAAGAGACAG | |
| UMI Bridged Primer | iSp18 = an 18-atom hexa-ethyleneglycol | |
| for Insert 1 Read | spacer between two oligonucleotides; may | |
| | be used for Illumina sequencing. | |
| Exemplary A2′- | GCTGTTCTTGAGTGACCGAGCCCACGAGAC | 10 |
| B15′ Custom i7 | | |
| Read | | |
| Exemplary A2′- | GCTGTTCTTGAGTGAGACGCTGCCGACGA | 11 |
| A14′ Custom i5 | | |
| Read | | |
| Exemplary B15-A2 | GTCTCGTGGGCTCGGTCACTCAAGAACAGC | 12 |
| Custom UMI 2 Read | | |
| Exemplary B15-A2- | GTCTCGTGGGCTCGGTCACTCAAGAACAGC/iSp18//iSp18/ | 13 |
| spacer-ME Custom | AGATGTGTATAAGAGACAG | |
| Bridged Primer for | iSp18 = an 18-atom hexa-ethyleneglycol | |
| Insert 2 Read | spacer between two oligonucleotides; may | |
| | be used for Illumina sequencing. | |
| Exemplary P5 | AATGATACGGCGACCACCGAGAUCTACAC | 14 |
| oligonucleotide | | |
| (UDI/Nextera index | | |
| primer) | | |
| P7 oligonucleotide | CAAGCAGAAGACGGCATACGAG*AT | 15 |
| (UDI/Nextera index | In some embodiments, G* indicates a guanine. | |
| primer) | In some embodiments, G* indicates a modified | |
| | guanine, e.g., an 8-oxo-guanine. | |

### DESCRIPTION OF THE EMBODIMENTS

### I. Definitions

“Hybridization sequence” or “HYB,” as used herein, refers to a sequence that can hybridize to a complementary hybridization sequence. Hybridization of HYB in one library product to a HYB′ in another library product can lead to a hybridization adduct, wherein the two library products anneal to each other via hybridization of HYB/HYB′.

“Hyb2Y” or “Hyb2Y workflow,” as used herein, refers to the use of HYB/HYB′ to produce a forked adapter structure (also known as a Y-adapter structure). In some instances, but not all, this process also involves replacing one oligonucleotide with another oligonucleotide.

In the context of bead linked transposomes (BLTs), “Hyb2Y,” i.e., using HYB/HYB′ to produce a forked adapter structure, results in removing the nontransferred strand from a Tn5 transposome product complex and replacing it with another oligonucleotide that may contain additional sequences to the oligonucleotide that it replaces. In doing so, one may create a new or maintain an existing forked architecture of an adapter being used.

“Insert sequence,” as used herein, refers to a region of a target nucleic acid that is comprised in a polynucleotide. A polynucleotide may comprise multiple insert sequences.

“Stacked reads,” as used herein, relates to sequencing reads of multiple insert sequences that are generated from a single polynucleotide. These sequencing reads may be sequential. For example, a polynucleotide comprising 2 or more insert sequences and 2 or more primer sequences can be used to generate stacked reads. A “stacked reads library,” as used herein, refers to a library of polynucleotides comprising multiple insert sequences that can be used to generate stacked reads.

“Sequencing-by-synthesis” or “SBS,” as used herein refers to a sequence that is incorporated into a polynucleotide to improve binding of a read primer. In embodiments wherein polynucleotides are made from library products produced by tagmentation, SBS may be a mosaic end sequence and SBS' may be the complement of a mosaic end sequence, such as ME and ME′. SBS and SBS' sequences may also be comprised in adapters when library products are produced using TruSeq™ methods (Illumina).

### II. Preparing UMI Libraries Using Transposon Based Technology

Unique Molecular Identifiers (UMIs) are nucleic acid sequences that are incorporated into double-stranded nucleic acid libraries for identifying and correcting sequencing errors and PCR duplicates. UMIs are used to distinguish one source DNA molecule from another when many DNA molecules are sequenced together. UMIs can be useful in helping to identify sequencing and PCR artifacts, and errors from strand-specific DNA damage such as those typically found in formalin-fixed, paraffin-embedded, FFPE, tissues. UMIs allow for the reduction of noise from errors that occur during PCR amplification and sequencing, enabling the detection of single nucleotide variants (SNVs) (in cell-free DNA, cfDNA, for example) at allele frequencies of <1%.

The materials and methods described herein may be used with transposon-based technology to incorporate UMIs into double-stranded nucleic acid libraries. As used herein, a “UMI library” is a library of double-stranded nucleic acid fragments wherein each fragment comprises at least one UMI. In certain embodiments described herein, each fragment may comprise one, two, or more UMIs.

Disclosed herein are approaches for generating sequencing libraries that are combined with transposon-based technology. In some embodiments, the transposon-based technology comprises a workflow for DNA Prep suite of products by Illumina® to produce a population of double-stranded nucleic acid fragments tagged with unique adapter sequences at the ends of the fragments. A variety of HYB or HYB′ sequences are disclosed for use in transposition reactions. In some embodiments, the methods are performed in a solution mixture. In some embodiments, a solid support such as BLTs are used.

In many embodiments, a method of preparing a UMI library comprises a first step of applying a sample with double-stranded target nucleic acids to one, two, or more transposome complexes.

In some embodiments, after the first step, the method of preparing a UMI library further comprises (1) tagmenting the nucleic acids to produce nucleic acid fragments comprising UMIs and adapter sequences, (2) releasing the nucleic acid fragments from the transposome complexes, (3) ligating the transposons or extended transposons with the nucleic acid fragments, (4) producing the nucleic acid fragments comprising the UMIs. In some embodiments, the method further comprises an optional extending step after the releasing step, wherein the double-stranded target nucleic acid fragments are extended. This extending step is also known as gap-filling.

In some embodiments, after the first step, the method of preparing a UMI library further comprises (1) tagmenting the nucleic acids to produce nucleic acid fragments comprising adapter sequences, (2) releasing the nucleic acid fragments from the transposome complexes, (3) hybridizing a polynucleotide comprising an adapter sequence and a UMI for incorporation of the UMI. The polynucleotide further comprises a sequence completely or partially complementary to a 3′ end transposon sequence. The method may further comprise an optional step where a second strand of a double-stranded target nucleic acid fragment is extended. The method may further comprise an optional step where the polynucleotide or extended polynucleotide is ligated. In some embodiments, method further comprises producing double-stranded target nucleic caid fragments with UMIs, wherein the UMI is located directly adjacent to the 3′ end of the insert DNA.

In some embodiments, after the first step, the method of preparing a UMI library further comprises (1) tagmenting a first strand of the double-stranded target nucleic acids with the transposon to produce double-stranded target nucleic acid fragments comprising a first adapter sequence, (2) releasing the double-stranded target nucleic acid fragments from the transposome complex, and (3) hybridizing a first polynucleotide comprising a UMI and a second adapter sequence. In some embodiments, the method may further comprise optional steps for (1) adding a second polynucleotide comprising regions complementary to the first polynucleotide to produce a double-stranded adapter, (2) extending a second strand of the double-stranded target nucleic acid fragments, and/or (3) optionally ligating the double-stranded adapter with the double-stranded target nucleic acid fragments.

In some embodiments, after the first step, the method of preparing a UMI library further comprises (1) tagmenting double-stranded target nucleic acids with forked adapter transposons to produce double-stranded target nucleic acid fragments comprising first and second copies of a first adapter sequence, a first UMI, first and second copies of a second adapter sequence, and a second UMI; (2) releasing the double-stranded target nucleic acid fragments from transposome complexes; and (3) ligating the forked adapter transposons with double-stranded target nucleic acid fragments. In some embodiments, after the releasing step, double-stranded target nucleic acid fragments are extended, in which case, the ligating step that follows ligates the extended forked adapter transposons with the double-stranded target nucleic acid fragments.

In many embodiments, after the UMI library is produced, the method further comprises amplifying the UMI library.

In some embodiments, the UMIs are incorporated during tagmentation using transposon adapters. In some embodiments, the UMIs are incorporated after tagmentation using polynucleotide adapters. In some embodiments, the UMIs are incorporated by extending and/or ligating polynucleotide adapters. In some embodiments, the UMIs are incorporated prior to library amplification.

Aspects for each of these steps are discussed in the sections that follow.

A. Unique Molecular Identifiers (UMIs)

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to bar codes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

The UMIs may be single or double-stranded, and may be at least 5 bases, at least 6 bases, at least 7 bases, at least 8 bases, or more. In certain embodiments, the UMIs are 5-8 bases, 5-10 bases, 5-15 bases, 5-25 bases, 8-10 bases, 8-12 bases, 8-15 bases, or 8-25 bases in length, etc. Further, in certain embodiments, the UMIs are no more than 30 bases, no more than 25 bases, no more than 20 bases, no more than 15 bases in length. It should be understood that the length of the UMI sequences as provided herein may refer to the unique/distinguishable portions of the sequences and may exclude adjacent common or adapter sequences (e.g., p5, p7) that may serve as sequencing primers and that are common between multiple UMIs having different identifier sequences.

UMIs may be defined in many ways, such as described in WO 2018/136248, which is incorporated herein by reference. UMIs maybe random, pseudo-random or partially random, or nonrandom nucleotide sequences that are inserted in adapters or otherwise incorporated in source DNA molecules to be sequenced. In some embodiments, the UMIs are unique that each UMI is able to provide unique identification for any given source DNA molecule present in a sample. As described herein, transposon adapters and polynucleotide adapters may be used to incorporate UMIs into target nucleic acids to be sequenced, and the individual sequenced molecules each has a UMI that helps distinguish it from all other fragments. In some embodiments, a large number of different physical UMIs may be used to uniquely identify DNA fragments in a sample. In some embodiments, the UMI is of a sufficient length to ensure uniqueness for each and every source DNA molecule.

In some embodiments, the library of UMIs comprises nonrandom sequences. In some embodiments, nonrandom UMIs (nrUMIs) are predefined for a particular experiment or application. In certain embodiments, rules are used to generate sequences for a set or select a sample from the set to obtain a nrUMI. For instance, the sequences of a set may be generated such that the sequences have a particular pattern or patterns. In some implementations, each sequence differs from every other sequence in the set by a particular number of (e.g., 2, 3, or 4) nucleotides. That is, no nrUMI sequence can be converted to any other available nrUMI sequence by replacing fewer than the particular number of nucleotides. In some implementations, a set of UMIs used in a sequencing process includes fewer than all possible UMIs given a particular sequence length. For instance, a set of nrUMIs having 6 nucleotides may include a total of 96 different sequences, instead of a total of 4\hat{ }6=4096 possible different sequences. In some embodiments, the library of UMIs comprises 120 nonrandom sequences.

In some implementations where nrUMIs are selected from a set with fewer than all possible different sequences, the number of nrUMIs is fewer, sometimes significantly so, than the number of source DNA molecules. In such implementations, nrUMI information may be combined with other information, such as virtual UMIs, read locations on a reference sequence, and/or sequence information of reads, to identify sequence reads deriving from a same source DNA molecule.

A “virtual unique molecular index” or “virtual UMI” is a unique subsequence in a source DNA molecule. In some implementations, virtual UMIs are located at or near the ends of the source DNA molecule. One or more such unique end positions may alone or in conjunction with other information uniquely identify a source DNA molecule. Depending on the number of distinct source DNA molecules and the number of nucleotides in the virtual UMI, one or more virtual UMIs can uniquely identify source DNA molecules in a sample. In some cases, a combination of two virtual unique molecular identifiers is required to identify a source DNA molecule. Such combinations may be extremely rare, possibly found only once in a sample. In some cases, one or more virtual UMIs in combination with one or more physical UMIs may together uniquely identify a source DNA molecule. In some embodiments, the virtual UMI reside at fragmentation end points that are derived from the Nextera fragmentation process.

In some embodiments, the library of UMIs may comprise random UMIs (rUMIs) that are selected as a random sample, with or without replacement, from a set of UMIs consisting of all possible different oligonucleotide sequences given one or more sequence lengths. For instance, if each UMI in the set of UMIs has n nucleotides, then the set includes 4\hat{ }n UMIs having sequences that are different from each other. A random sample selected from the 4\hat{ }n UMIs constitutes a rUMI.

In some embodiments, the library of UMIs is pseudo-random or partially random, which may comprise a mixture of nrUMIs and rUMIs.

In many embodiments, UMIs are added to target double stranded nucleic acids using oligonucleotides or polynucleotides during or after tagmentation of said nucleic acids. In many embodiments, UMIs are added to target double stranded nucleic acids before the library amplification step.

In some embodiments, UMI reagents from the TruSight® Oncology workflow (Illumina Catalog #20024586) may be utilized in accordance with the present disclosure.

In some embodiments, the double stranded nucleic acid molecules in a UMI library each comprises one unique UMI sequence, or single UMI. In many embodiments, the UMI may be located on either side of the insert DNA. In some embodiments, adapter sequences or other nucleotide sequences may be present between the UMI and the insert DNA.

In some embodiments, the UMI library comprises duplex UMI, which may lower the limit of error detection as compared to the use of a single UMI. Duplex UMIs enable a skilled artisan to pair a plus strand with its minus strand despite errors that may arise in a sequencing reaction. Such sequencing mismatches are identified during sequencing, and the sequence of a nucleic acid fragment can still be correctly reconstituted despite having mismatches. In some embodiments, a method of producing a UMI library comprising duplex UMI comprises forked adapters, as discussed in detail in Section II.C below. In some embodiments, the forked adapters are BLT fork adapters.

In some embodiments, each double-stranded nucleic acid fragment in the UMI library comprises two, three or four UMI sequences. The UMI sequences may have complementary sequences with each other or may each have a different sequence.

In some embodiments, adapter sequences or other nucleotide sequences may be present between each UMI and the insert DNA.

In some embodiments, the UMI is located 5′ of the insert DNA. In some embodiments, the UMI is located 3′ of the insert DNA. In some embodiments, a sequence of nucleic acids representing one or more adapter sequences may be located between the UMI and the insert DNA. In some embodiments, the UMI is located between an adapter sequence and a transposon end sequence

In many embodiments, the UMI can be on the first strand, second strand, or both strands of the double-stranded target nucleic acid fragments. In some embodiments, the UMI is on the first strand. In some embodiments, a first copy of the UMI is on the first strand and a second copy of the UMI is on the second strand of the double-stranded target nucleic acid fragments. In some embodiments, a first UMI is on a first strand and a second UMI is on a second strand.

1. In-Line UMIs

A UMI may be located anywhere on a double stranded nucleic acid molecule. In many embodiments, the location of a UMI on a double stranded nucleic acid molecule will vary. In some embodiments, the UMI is located directly adjacent to the insert DNA, i.e., the UMI is an “in-line UMI.” In some embodiments, the in-line UMI is adjacent to the 3′ end of the insert DNA. In some embodiments, the in-line UMI is adjacent to the 5′ end of the insert DNA. Current BLT approaches contain an ME adjacent to target inserts, which precludes the use of Illumina ligation adapters with UMIs. While UMIs are useful for removing PCR duplicates in double-stranded nucleic acids and for detection of low-frequency variants, UDIs are useful for mitigating sample misassignment due to index hopping in library sequencing and demultiplexing. UDIs are unique i5 and i7 index sequences that are added to the ends of target nucleic acids so that both ends contain a UDI. UDIs are used with patterned flow cells, such as Illumina's NovaSeq 6000 system (See, e.g., WO 2018/204423, WO 2018/208699, WO 201/9055715, and WO 2016/176091; which are incorporated by reference herein in their entireties). One skilled in the art would appreciate that in-line UMIs allow for the compatibility of UMI libraries with standard, downstream library preparations that utilize UDIs, such as sample multiplexing PCR and sequencing chemistry recipes in Illumina's TruSeq™ and AmpliSeq™ workflows. In some embodiments, the sequencing methods used with in-line UMIs do not require custom primers or custom reads.

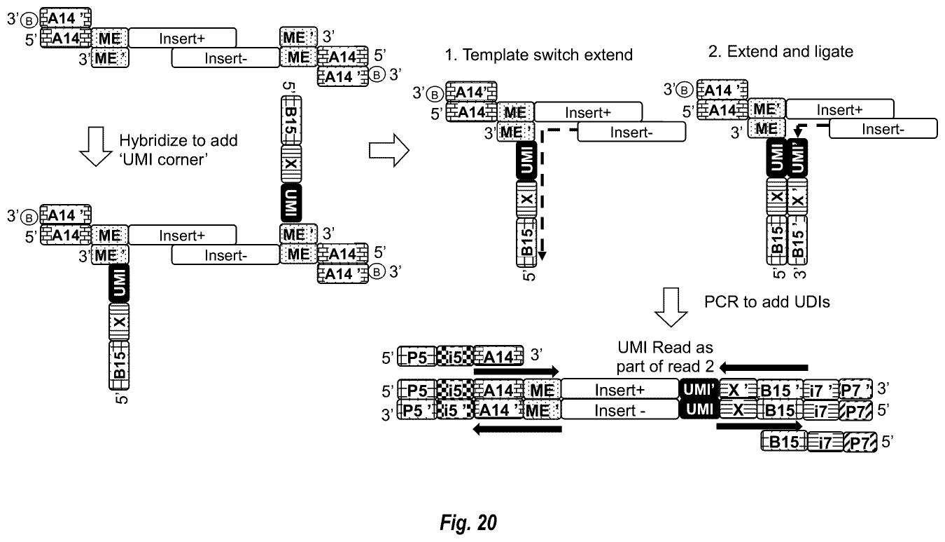

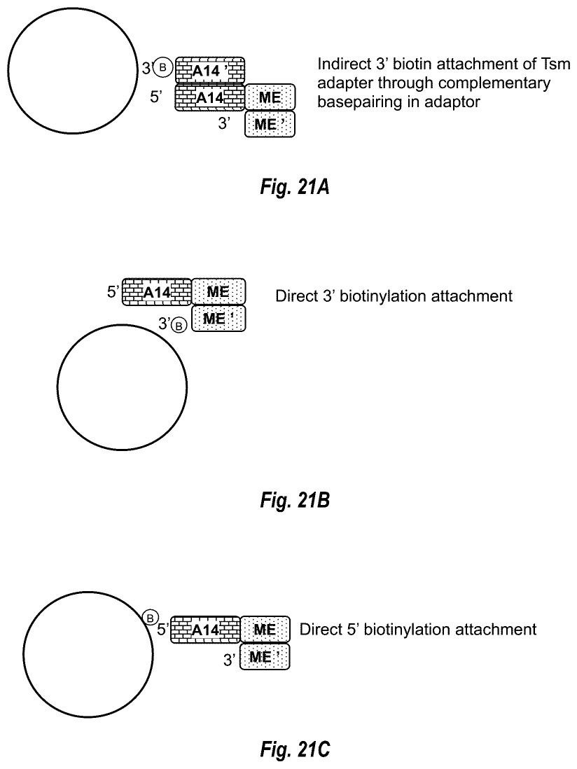

In some embodiments, a standard sequencing method is used to sequence a UMI library with in-line UMIS. In these embodiments, the UMI is adjacent to the 3′ end of the insert nucleic acids (FIG. 20). As such, each UMI and insert nucleic acid sequence is captured using Read 2 without having to sequence an ME sequence in between them. In these embodiments, the sequencing method does not comprise dark cycles. Dark cycles are discussed in Section III.A below.

In some embodiments, the “in-line UMI” is located between the insert DNA and an adapter sequence. In some embodiments, the adapter sequence is a second adapter sequence.

B. Transposome Complexes

Generally, the present transposon complexes comprise a transposase and a first and second transposon, along with one or more components that mediate targeting to one or more nucleic acid sequence of interest.

A “transposome complex,” as used herein, is comprised of at least one transposase (or other enzyme as described herein) and a transposon recognition sequence. In some such systems, the transposase binds to a transposon recognition sequence to form a functional complex that is capable of catalyzing a transposition reaction. In some aspects, the transposon recognition sequence is a double-stranded transposon end sequence. The transposase binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting in a cleavage event. Exemplary transposition procedures and systems that can be readily adapted for use with the transposases.

In some embodiments, the methods comprise one, two, or more transposome complexes. Each transposome complex may comprise a transposase and transposons which are different from other transposome complexes that may also be used in the same method.

In some embodiments, a transposome complex comprises a transposase and one, two or more transposons.

In some embodiments, a transposome complex comprises a transposase and a first transposon comprising a 3′ transposon end sequence and a 5′ adapter sequence. The 5′ adapter sequence of the first transposon may comprise an A14 sequence (SEQ ID NO: 4), an A2 sequence (SEQ ID NO: 7), and/or a B15 sequence (SEQ ID NO: 5). In some embodiments, the first transposon also comprises a UMI sequence.

In some embodiments, the transposome complex also comprises a first and a second transposon. The second transposon comprises a 5′ transposon end sequence. The 5′ transposon end sequence of the second transposon may be complementary to the 3′ transposon end sequence of the first transposon.

In some embodiments, the second transposon also comprises a 3′ adapter sequence. The 3′ adapter sequence of the second transposon may be partially or completely complementary to the 5′ adapter sequence of the first transposon.

In some embodiments, 3′ adapter sequence of the second transposon contains no portion that is complementary to the 5′ adapter sequence of the first transposon.

In some embodiments, the 3′ adapter sequence of the second transposon comprises an A14 sequence (SEQ ID NO: 4), an A2 sequence (SEQ ID NO: 7), a B15 sequence (SEQ ID NO: 5), and/or a sequence that is complementary to the UMI sequence of the first transposon.

In some embodiments, the second transposon further comprises a UMI. The UMI of the second transposon may be the same sequence or a different sequence from the UMI of the first transposon.

In some embodiments, the transposome complex comprises one, two, or more transposons, each with a sequence comprising A14-ME (SEQ ID NO: 1), and/or B15-ME (SEQ ID NO: 2).

In some embodiments, the transposon complex comprises a first transposon with a 3′ transposon end sequence comprising ME (SEQ ID NO: 6) or ME′ (SEQ ID NO: 3). In some embodiments, the transposon complex comprises a second transposon with a 3′ transposon end sequence comprising ME (SEQ ID NO: 6) or ME′ (SEQ ID NO: 3).

In some embodiments, the transposome complex comprises an additional adapter sequence adjacent to an A14 sequence (SEQ ID NO: 4), an A2 sequence (SEQ ID NO: 7), a B15 sequence (SEQ ID NO: 5), an ME sequence (SEQ ID NO: 6), and/or a ME′ sequence (SEQ ID NO: 3). Many sequences may be used as an additional adapter sequence, such as those disclosed in in Illumina Adapter Sequences Document #1000000002694 v15, which is incorporated herein by reference. In some embodiments, the additional adapter sequence is an A adapter sequence, a B adapter sequence, a X adapter sequence, or a Y′ adapter sequence.

In some embodiments, the transposome complex comprises an oligonucleotide complementary to the B15 sequence and/or the A14 sequence.

In some embodiments, the transposome complex is immobilized to solid support, such as a bead or other material. In some embodiments, the transposome complex is immobilized via the first or second transposon. In some embodiments, the transposome complex is immobilized via an oligonucleotide that is complementary to an adapter sequence (such as a B15 sequence or an A14 sequence) of the first or second transposon.

1. Transposase

A “transposase” means an enzyme that is capable of forming a functional complex with a transposon end-containing composition (e.g., transposons, transposon ends, transposon end compositions) and catalyzing insertion or transposition of the transposon end-containing composition into a double-stranded target nucleic acid. A transposase as presented herein can also include integrases from retrotransposons and retroviruses.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising RI and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. More examples include IS5, Tn10, Tn903, IS911, and engineered versions of transposase family enzymes. The methods described herein could also include combinations of transposases, and not just a single transposase.

In some embodiments, the transposase is a Tn5, Tn7, MuA, or Vibrio harveyi transposase, or an active mutant thereof In other embodiments, the transposase is a Tn5 transposase or a mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, which is incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998). In one embodiment, a transposase recognition site that forms a complex with a hyperactive Tn5 transposase is used (e.g., EZ-Tn5TM Transposase, Epicentre Biotechnologies, Madison, Wis.). In some embodiments, the Tn5 transposase is a wild-type Tn5 transposase.

As used throughout, the term transposase refers to an enzyme that is capable of forming a functional complex with a transposon-containing composition (e.g., transposons, transposon compositions) and catalyzing insertion or transposition of the transposon-containing composition into the double-stranded target nucleic acid with which it is incubated in an in vitro transposition reaction. A transposase of the provided methods also includes integrases from retrotransposons and retroviruses. Exemplary transposases that can be used in the provided methods include wild-type or mutant forms of Tn5 transposase and MuA transposase.

A “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The method of this disclosure is exemplified by employing a transposition complex formed by a hyperactive Tn5 transposase and a Tn5-type transposon end or by a MuA or HYPERMu transposase and a Mu transposon end comprising RI and R2 end sequences (See e.g., Goryshin, I. and Reznikoff, W. S., J. Biol. Chem., 273: 7367, 1998; and Mizuuchi, Cell, 35: 785, 1983; Savilahti, H, et al., EMBO J., 14: 4893, 1995; which are incorporated by reference herein in their entireties). However, any transposition system that is capable of inserting a transposon end in a random or in an almost random manner with sufficient efficiency to tag target nucleic acids for its intended purpose can be used in the provided methods. Other examples of known transposition systems that could be used in the provided methods include but are not limited to Staphylococcus aureus Tn552, Ty1, Transposon Tn7, Tn/O and IS 10, Mariner transposase, Tel, P Element, Tn3, bacterial insertion sequences, retroviruses, and retrotransposon of yeast (See, e.g., Colegio O R et al, J. Bacteriol., 183: 2384-8, 2001; Kirby C et al, Mol. Microbiol., 43: 173-86, 2002; Devine S E, and Boeke J D., Nucleic Acids Res., 22: 3765-72, 1994; International Patent Application No. WO 95/23875; Craig, N L, Science. 271: 1512, 1996; Craig, N L, Review in: Curr Top Microbiol Immunol., 204: 27-48, 1996; Kleckner N, et al., Curr Top Microbiol Immunol., 204: 49-82, 1996; Lampe D J, et al., EMBO J., 15: 5470-9, 1996; Plasterk R H, Curr Top Microbiol Immunol, 204: 125-43, 1996; Gloor, G B, Methods Mol. Biol, 260: 97-1 14, 2004; Ichikawa H, and Ohtsubo E., J Biol. Chem. 265: 18829-32, 1990; Ohtsubo, F and Sekine, Y, Curr. Top. Microbiol. Immunol. 204: 1-26, 1996; Brown P O, et al, Proc Natl Acad Sci USA, 86: 2525-9, 1989; Boeke J D and Corces V G, Annu Rev Microbiol. 43: 403-34, 1989; which are incorporated herein by reference in their entireties).

The method for inserting a transposon into a target sequence can be carried out in vitro using any suitable transposon system for which a suitable in vitro transposition system is available or can be developed based on knowledge in the art. In general, a suitable in vitro transposition system for use in the methods of the present disclosure requires, at a minimum, a transposase enzyme of sufficient purity, sufficient concentration, and sufficient in vitro transposition activity and a transposon with which the transposase forms a functional complex with the respective transposase that is capable of catalyzing the transposition reaction. Suitable transposase transposon end sequences that can be used include but are not limited to wild-type, derivative or mutant transposon end sequences that form a complex with a transposase chosen from among a wild-type, derivative or mutant form of the transposase.

In some embodiments, the transposase comprises a Tn5 transposase. In some embodiments, the Tn5 transposase is hyperactive Tn5 transposase.

In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adapter sequence in each monomer and the second population has a different adapter sequence in each monomer.

The term “transposon end” refers to a double-stranded nucleic acid molecule that exhibits only the nucleotide sequences (the “transposon end sequences”) that are necessary to form the complex with the transposase or integrase enzyme that is functional in an in vitro transposition reaction. In some embodiments, the double-stranded nucleic acid molecule is DNA. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

2. Transferred Strand and Non-Transferred Strand

The term “transferred strand” refers to the transferred portion of both transposon ends. Similarly, the term “non-transferred strand” refers to the non-transferred portion of both “transposon ends.” The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure. Additional examples of transposome structure and methods of preparing and using transposomes can be found in the disclosure of US 2010/0120098, the content of which is incorporated herein by reference in its entirety.

In some embodiments, the transposome complexes comprise a first transposon comprising a 3′ transposon end sequence and a 5′ adapter sequence. In some embodiments, the transposome complexes comprise a second transposon comprising a 5′ transposon end sequence, wherein the 5′ transposon end sequence is complementary to the 3′ transposon end sequence.

Thus, in some embodiments, the tagmenting step produces double-stranded target nucleic acid fragments comprising: (1) a first strand comprising a first adapter sequence and a first UMI, and (2) a second strand comprising a second adapter sequence. In some embodiments, the second strand may further comprise a second UMI.

3. Tagmentation

“Tagmentation,” as used herein, refers to the use of transposase to fragment and tag nucleic acids. Tagmentation includes the modification of nucleic acids by a transposome complex comprising transposase enzyme complexed with one or more adapter sequences comprising transposon end sequences (referred to herein as transposons). Tagmentation thus can result in the simultaneous fragmentation of the DNA and ligation of the adapters to the 5′ ends of both strands of duplex fragments.

In many embodiments, tagmentation may comprise a plurality of transposome complexes, each comprising a transposase complexed with a transposon comprising a transposon end sequence and an adapter sequence. In some embodiments, the tagmentation is symmetric tagmentation wherein all the adapter sequences in the plurality of transposome complexes are identical. In some embodiments, the tagmentation is standard or asymmetric tagmentation wherein the plurality of transposome complexes comprise two different sets of adapter sequences. Adapter sequences are discussed in Section II.C below. Symmetric tagmentation and asymmetric tagmentation are described in WO 2015/168161 and WO 2017/040306, which are incorporated by reference in their entireties herein.

In some embodiments, a method comprises a first transposase, a first transposon, and a second transposon. In some embodiments, the method further comprises a second transposase, a third transposon, and a fourth transposon.

In many embodiments, the tagmenting step produces double-stranded target nucleic acid fragments with adapter sequences and/or UMIs which can be arranged in several ways. The location of adapter sequences and UMIs (or the order of adapter sequences and UMIs from 5′ to 3′) depend on the transposon adapters used in the tagmentation. In some embodiments, the tagmenting step produces double-stranded target nucleic acid fragments comprising a first adapter sequence and a first UMI. In some embodiments, the first adapter sequence and first UMI are on the first strand of nucleic acid fragments.

In some embodiments, the tagmenting step produces double-stranded target nucleic acid fragments comprising a first adapter sequence, a first UMI, and a second adapter sequence. In some embodiments, the first adapter sequence and first UMI are on the first strand of nucleic acid fragments while the second adapter sequence is on the second strand of nucleic acid fragments.

In some embodiments, the tagmenting step produces double-stranded comprising a first adapter sequence, a first UMI, a second adapter sequence, and a second UMI. In some embodiments, the first adapter sequence and first UMI are on the first strand of nucleic acid fragments while the second adapter sequence and the second UMI are on the second strand of nucleic acid fragments.

In some embodiments, the tagmenting step produces double-stranded target nucleic acids with forked adapter transposons to produce double-stranded target nucleic acid fragments comprising the first and second copies of the first adapter sequence, the first UMI, the first and second copies of the second adapter sequence, and the second UMI.

In some embodiments, the tagmenting step produces double-stranded target nucleic acid fragments further comprising a third UMI and/or a fourth UMI.

In some embodiments, the tagmenting step produces double-stranded target nucleic acids comprising one or more adapter sequences without any UMIs. In some embodiments, the one or more adapter sequences is on the first strand of nucleic acid fragments.

4. Immobilized Transposome Complexes

A number of different types of immobilized transposomes can be used in these methods, as described in U.S. Pat. No. 9,683,230, which is incorporated herein in its entirety. In the methods and compositions presented herein, transposome complexes are immobilized to the solid support. In some embodiments, the transposome complexes and/or capture oligonucleotides are immobilized to the support via one or more polynucleotides, such as a polynucleotide comprising a transposon end sequence. In some embodiments, the transposome complex may be immobilized via a linker molecule coupling the transposase enzyme to the solid support. In some embodiments, both the transposase enzyme and the polynucleotide are immobilized to the solid support. When referring to immobilization of molecules (e.g., nucleic acids) to a solid support, the terms “immobilized” and “attached” are used interchangeably herein and both terms are intended to encompass direct or indirect, covalent or non-covalent attachment, unless indicated otherwise, either explicitly or by context. In some embodiments, covalent attachment may be used, but generally all that is required is that the molecules (e.g., nucleic acids) remain immobilized or attached to the support under the conditions in which it is intended to use the support, for example in applications requiring nucleic acid amplification and/or sequencing.

In some embodiments, the transposomes are immobilized using transposons comprising a biotin tag.

In some embodiments, the transposome complexes are present on the solid support at a density of at least 10³, 10⁴, 10⁵, or 10⁶ complexes per mm².

In some embodiments, the lengths of the double-stranded fragments in the immobilized library are adjusted by increasing or decreasing the density of transposome complexes on the solid support.

a) Capture Oligonucleotides

In some embodiments, capture oligonucleotides are immobilized on a solid support.

In some embodiments, the 3′ end of the target DNA binds to the capture oligonucleotides.

In some embodiments, the 3′ end of the target RNA binds to the capture oligonucleotides. In some embodiments, capture oligonucleotides may serve to immobilize the target RNA on the solid support.

In some embodiments, the capture oligonucleotides comprise a polyT sequence.

In some embodiments, the target RNA is mRNA, and the mRNA binds to capture oligonucleotides comprising polyT sequences.

In some embodiments, the capture oligonucleotides do not comprise polyT sequences.

In some embodiments, the capture oligonucleotides are immobilized to the beads via P5 or P7 sequences.

In some embodiments, the capture oligonucleotides comprise a tag that is also present in the first tag comprised in the first polynucleotide of the immobilized transposomes.

b) Solid Supports

Certain embodiments may make use of solid supports comprised of an inert substrate or matrix (e.g., glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (polynucleotides) may be directly covalently attached to the intermediate material (e.g., the hydrogel) but the intermediate material may itself be non-covalently attached to the substrate or matrix (e.g., the glass substrate). The term “covalent attachment to a solid support” is to be interpreted accordingly as encompassing this type of arrangement.

The terms “solid surface,” “solid support” and other grammatical equivalents herein refer to any material that is appropriate for or can be modified to be appropriate for the attachment of the transposome complexes. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flow cell apparatus. Exemplary flow cells are set forth in further detail below.

In some embodiments, the solid support comprises a patterned surface suitable for immobilization of transposome complexes in an ordered pattern. A “patterned surface” refers to an arrangement of different regions in or on an exposed layer of a solid support. For example, one or more of the regions can be features where one or more transposome complexes are present. The features can be separated by interstitial regions where transposome complexes are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the transposome complexes are randomly distributed upon the solid support. In some embodiments, the transposome complexes are distributed on a patterned surface. Exemplary patterned surfaces that can be used in the methods and compositions set forth herein are described in U.S. Ser. No. 13/661,524 or US 2012/0316086 A1, each of which is incorporated herein by reference.

In some embodiments, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flow cell. The term “flow cell” as used herein refers to a chamber comprising a solid surface across which one or more fluid reagents can be flowed. Examples of flow cells and related fluidic systems and detection platforms that can be readily used in the methods of the present disclosure are described, e.g., in Bentley et al., Nature 456:53-59 (2008), WO 2004/018497; U.S. Pat. No. 7,057,026; WO 1991/06678; WO 2007/123744; U.S. Pat. Nos. 7,329,492; 7,211,414; 7,315,019; 7,405,281, and US 2008/0108082, each of which is incorporated herein by reference.

In some embodiments, the solid support or its surface is non-planar, such as the inner or outer surface of a tube or vessel. In some embodiments, the solid support comprises microspheres or beads. By “microspheres” or “beads” or “particles” or grammatical equivalents herein is meant small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Selection Guide” from Bangs Laboratories, Fishers Ind. is a helpful guide. In certain embodiments, the microspheres are magnetic microspheres or beads.

The beads need not be spherical; irregular particles may be used. Alternatively or additionally, the beads may be porous. The bead sizes range from nanometers, i.e., 100 nm, to millimeters, i.e., 1 mm, with beads from 0.2 micron to 200 microns, or from 0.5 to 5 microns, although in some embodiments smaller or larger beads may be used.

The density of these surface bound transposomes can be modulated by varying the density of the first polynucleotide or by the amount of transposase added to the solid support. For example, in some embodiments, the transposome complexes are present on the solid support at a density of at least 103, 104, 105, or 106 complexes per mm².

Attachment of a nucleic acid to a support, whether rigid or semi-rigid, can occur via covalent or non-covalent linkage(s). Exemplary linkages are set forth in U.S. Pat. Nos. 6,737,236; 7,259,258; 7,375,234 and 7,427,678; and US No. 2011/0059865 A1, each of which is incorporated herein by reference. In some embodiments, a nucleic acid or other reaction component can be attached to a gel or other semisolid support that is in turn attached or adhered to a solid-phase support. In such embodiments, the nucleic acid or other reaction component will be understood to be solid-phase.

In some embodiments, the solid support comprises microparticles, beads, a planar support, a patterned surface, or wells. In some embodiments, the planar support is an inner or outer surface of a tube.

In some embodiments, a solid support has a library of tagged DNA fragments immobilized thereon prepared.

In some embodiments, solid support comprises capture oligonucleotides and a first polynucleotide immobilized thereon, wherein the first polynucleotide comprises a 3′ portion comprising a transposon end sequence and a first tag.

In some embodiments, the solid support further comprises a transposase bound to the first polynucleotide to form a transposome complex.

In some embodiments, a solid support comprises capture oligonucleotides and a second polynucleotide immobilized thereon, wherein the second polynucleotide comprises a 3′ portion comprising a transposon end sequence and a second tag.

In some embodiments, the solid support further comprises a transposase bound to the second polynucleotide to form a transposome complex.

In some embodiments, a kit comprises a solid support as described herein. In some embodiments, a kit further comprises a transposase. In some embodiments, a kit further comprises a reverse transcriptase polymerase. In some embodiments, a kit further comprises a second solid support for immobilizing DNA.

5. Solution-Phase Transposome Complexes

Transposome complexes may be solution-phase transposome complexes. These solution-phase transposome complexes may be mobile and not immobilized to a solid support. In some embodiments, solution-phase transposome complexes are used to generate tagged fragments in solution.

Further, present methods may comprise steps involving solution-phase transposome complexes. For example, a method presented herein can further comprise a step of providing transposome complexes in solution and contacting the solution-phase transposome complexes with the immobilized fragments under conditions whereby the DNA is fragmented by the transposome complexes solution; thereby obtaining immobilized nucleic acid fragments having one end in solution. In some embodiments, the transposome complexes in solution can comprise a second tag, such that the method generates immobilized nucleic acid fragments having a second tag, the second tag in solution. The first and second tags can be different or the same.

In some embodiments, the method further comprises contacting solution-phase transposome complexes with double-stranded nucleic acids under conditions whereby the DNA fragments are further fragmented by the solution-phase transposome complexes; thereby obtaining immobilized nucleic acid fragments having one end in solution.

In some embodiments, the solution-phase transposome complexes comprise a second tag, thereby generating immobilized nucleic acid fragments having a second tag in solution. In some embodiments, the first and second tags are different. In some embodiments, at least 50%, 55%, 60%, 65%, 70%, 75%, 80%, 85%, 90%, 95%, 96%, 97%, 98%, or 99% of the solution-phase transposome complexes comprise a second tag.

In some embodiments, one form of surface bound transposome is predominantly present on the solid support. For example, in some embodiments, at least 50%, 55%, 60%, 65%, 70%, 75%, 80%, 85%, 90%, 95%, 96%, 97%, 98%, or 99% of the tags present on said solid support comprise the same tag domain. In such embodiments, after an initial tagmentation reaction with surface bound transposomes, at least 50%, 55%, 60%, 65%, 70%, 75%, 80%, 85%, 90%, 95%, 96%, 97%, 98%, or 99% of the bridge structures comprise the same tag domain at each end of the bridge. A second tagmentation reaction can be performed by adding transposomes from solution that further fragment the bridges. In some embodiments, most or all of the solution phase transposomes comprise a tag domain that differs from the tag domain present on the bridge structures generated in a first tagmentation reaction. For example, in some embodiments, at least 50%, 55%, 60%, 65%, 70%, 75%, 80%, 85%, 90%, 95%, 96%, 97%, 98%, or 99% of the tags present in the solution phase transposomes comprise a tag domain that differs from the tag domain present on the bridge structures generated in the first tagmentation reaction.

In some embodiments, the length of the templates is longer than what can be suitably amplified using standard cluster chemistry. For example, in some embodiments, the length of templates is at least 100 bp, 200 bp, 300 bp, 400 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, 10000 bp, 30000 bp or 100,000 bp. In such embodiments, then a second tagmentation reaction can be performed by adding transposomes from solution that further fragment the bridges, as described in U.S. Pat. No. 9,683,230, which is incorporated herein in its entirety. The second tagmentation reaction can thus remove the internal span of the bridges, leaving short stumps anchored to the surface that can converted into clusters ready for further sequencing steps. In particular embodiments, the length of the template can be within a range defined by an upper and lower limit selected from those exemplified above.

C. Adapters

An “adapter” as used herein refers to a transposon or a polynucleotide that exhibits one or more “adapter sequences” for one or more desired intended purposes or applications. An adapter can comprise any sequence provided for any desired purpose.

An adapter may be a 5′ adapter or a 3′ adapter. A 5′ adapter is used with the intention of being ligated to the 5′ end of a target nucleic acid molecule. A 3′ adapter is with the intention of being ligated to the 3′ end of a target nucleic acid molecule.

In some embodiments, an adapter sequence comprises one or more regions suitable for hybridization with a primer for an amplification reaction. In some embodiments, an adapter sequence comprises one or more regions suitable for hybridization with a primer for a sequencing reaction. In some embodiments, an adapter sequence comprises one or more regions suitable for hybridization with a polynucleotide for incorporating UMI. In such embodiments, a HYB/HYB′ or Hyb2Y workflow may be used to incorporate the UMI.

In some embodiments, the adapter sequence comprises a UMI, a primer sequence, an index tag sequence, a capture sequence, a barcode sequence, a cleavage sequence, an anchor sequence, a universal sequence, a spacer region, a transposon end sequence, or a sequencing-related sequence, or a combination thereof. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adapter to nucleic acid fragments. In some embodiments, the adapter sequence comprises a P5 or P7 sequence (or their complement) to facilitate binding to a flow cell in certain sequencing methods. It will be appreciated that any other suitable feature can be incorporated into an adapter, and that adapter sequences may be used in any combination and arranged in any order from 5′ to 3′. In some embodiments, the transposon end sequence is a mosaic end sequence (ME).

An adapter may comprise one, two, or more read sequencing adapter sequences. In some embodiments, the adapter sequence is a 5′ first-read sequencing adapter sequence. In some embodiments, the adapter sequence is a 5′ second-read sequencing adapter sequence. In some embodiments, the first-read and/or second-read sequencing adapter sequences comprise unique primer binding sites.

In some embodiments, the adapter sequence comprises a sequence having a length from 5 bp to 200 bp. In some embodiments, the adapter sequence comprises a sequence having a length from 10 bp to 100 bp. In some embodiments, the adapter sequence comprises a sequence having a length from 20 bp to 50 bp. In some embodiments, the adapter sequence comprises a sequence having a length of 5, 6, 7, 8, 9, 10, 20, 30, 40, 50, 60, 70, 80, 90, 100, 150 or 200 bp.

While a variety of sequences may be used in an adapter, provided below are certain sequences which may be used in an adapter sequence, unique primer binding site, polynucleotide, or transposon end sequence (ME). The sequences may be used in any combination and may be arranged in an order from 5′ to 3′. Exemplary sequences for A14-ME, ME, B15-ME, ME′, A14, B15, and ME, are provided below:

A14-ME:
(SEQ ID NO: 1)
5′-TCGTCGGCAGCGTCAGATGTGTATAAGAGACAG-3′
B15-ME:
(SEQ ID NO: 2)
5′-GTCTCGTGGGCTCGGAGATGTGTATAAGAGACAG-3′
ME′:
(SEQ ID NO: 3)
5′-phos-CTGTCTCTTATACACATCT-3′
A14:
(SEQ ID NO: 4)
5′-TCGTCGGCAGCGTC-3′
B15:
(SEQ ID NO: 5)
5′-GTCTCGTGGGCTCGG-3′
ME:
(SEQ ID NO: 6)
AGATGTGTATAAGAGACAG
A2:
(SEQ ID NO: 7)
TCACTCAAGAACAGC

In some embodiments, the adapter sequence is incorporated during tagmentation. In these embodiments, a transposon with the adapter sequence is used in a tagmentation step.

In some embodiments, the adapter sequence is incorporated during an adapter ligation step. In these embodiments, a polynucleotide with the adapter sequence is used in a ligation step. In some embodiments, one, two, or more polynucleotides may be used.

1. Forked Adapters

In some embodiments, the adapter may be a forked adapter, also known as a Y-adapter. Forked adapter-based technology can be utilized for generating polynucleotides, for example, as exemplified in the workflow for TruSeq™ sample preparation kits (Illumina, Inc.). Reagents from the workflow for TruSight® Oncology kits (Illumina, Inc.) may also be used to assemble forked adapters. In many embodiments, a HYB/HYB′ workflow is used to produce a forked adapter.

As used herein, a “forked adapter” refers to an adapter comprising two strands of nucleic acid, wherein the two strands each comprise a region that is complementary to the other strand and a region that is not complementary to the other strand. In some embodiments, the two strands of nucleic acid in the forked adapter are annealed together before ligation, with the annealing based on complementary regions. In some embodiments, the complementary regions each comprise 12 nucleotides. In some embodiments, a forked adapter is ligated to both strands at the end of a double-stranded DNA fragment. In some embodiments, a forked adapter is ligated to one end of a double-stranded DNA fragment. In some embodiments, a forked adapter is ligated to both ends of a double-stranded DNA fragment. In some embodiments, the forked adapters on opposite ends of a fragment are different. In some embodiments, one strand of the forked adapter is phosphorylated at it 5′ to promote ligation to fragments. In some embodiments, one strand of the forked adapter has a phosphorothioate bond directly before a 3′ T. In some embodiments, the 3′ T is an overhang (i.e., not paired with a nucleotide in the other strand of the forked adapter). In some embodiments, the 3′ T overhang can base pair with an A-tail present on a library fragment. In some embodiments, the phosphorothioate bond blocks exonuclease digestion of the 3′ T overhang. In some embodiments, PCR with partially complementary primers is used after adapter ligation to extend ends and resolve the forks.

In some embodiments, the transposome complex has a structure of:

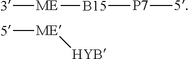

In some embodiments, the transposome complex has a structure of:

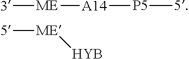

2. Transposon Adapters

In some embodiments, a UMI is incorporated during a tagmenting step. In these embodiments, the adapter used for incorporating UMI is a transposon. In some embodiments, the UMI is located between an adapter sequence and a 3′ transposon end sequence. In some embodiments, an adapter sequence is located between a UMI and 3′ end transposon end sequence. In some embodiments, adapter sequence may comprise a sequence that is completely or partially complementary to a 3′ end transposon end sequence.

In some embodiments, the transposon is a forked adapter transposon. A forked adapter may comprise two strands. In some embodiments, the first strand of the forked adapter transposon comprises a 3′ end transposon end sequence, an adapter sequence, and a UMI. In some embodiments, the second strand of the forked adapter transposon comprises an adapter sequence and a sequence completely or partially complementary to the first strand of the first forked adapter transposon. The sequence with full or partial complementarity in the first and second strands allow for the two strands to hybridize to form the forked structure.

In some embodiments, more than one forked adapter transposon may be used to incorporate more than one UMI and more than one adapter sequence into the library.

In some embodiments, two forked adapter transposons are used to incorporate two UMIs and four adapter sequences into the library. In some embodiments, tagmenting the double-stranded nucleic acids with the forked adapter transposons produces double-stranded target nucleic acid fragments with two UMIs, first and second copies of a first adapter sequence, and first and second copies of a second adapter sequence.

In some embodiments, two forked adapter transposons are used to incorporate four UMIs and four adapter sequences into the library. In some embodiments, tagmenting the double-stranded nucleic acids with forked adapter transposons produces double-stranded target nucleic acid fragments with four UMIs and four adapter sequences.

In some embodiments, the transposon further comprises one, two, three, four, or more unique primer binding sequences. In some embodiments, the unique primer binding sequences is used in a Hyb2Y workflow. In some embodiments, the unique primer binding sequence is used to anneal custom sequencing primers. In some embodiments, the unique primer binding sequence comprises A2, A14, and/or B15.

3. Polynucleotide Adapters

In some embodiments, a UMI is incorporated after tagmentation. In these embodiments, the adapter used to incorporate UMI is a polynucleotide. In some embodiments, the method comprises one, two, or more polynucleotides. In some embodiments, the polynucleotide comprises a UMI and one, two, or more adapter sequences. In some embodiments, the polynucleotide comprises regions for hybridizing via complementary sequence to other polynucleotides or transposons. For example, a polynucleotide may comprise a sequence completely or partially complementary to a 3′ end transposon sequence. In some embodiments, one or more polynucleotides are treated in a hybridizing step to generate a forked adapter.

In some embodiments, a portion of a polynucleotide may comprise a 3′ adapter. A 3′ adapter may comprise a hairpin UMI, a universal hybridizing tail, a splint ligation adapter, and/or a template switch oligonucleotide.

In some embodiments, the polynucleotide comprises a hairpin UMI. In some of these embodiments, the polynucleotide further comprises a universal hybridizing tail. In some embodiments, the hairpin UMI is stable during the extending and/or ligating step, but not during the amplifying step of the method. In some embodiments, the UMI comprises a 3 or 4 base pair stem. In some embodiments, the universal hybridizing tail comprises nucleotides, such as inosines, that can bind to any DNA molecule.

In some embodiments, the polynucleotide comprises a splint ligation adapter.

In some embodiments, the polynucleotide comprises a template switch oligonucleotide.

D. Extending and Ligating Steps after Tagmentation

In some embodiments, gaps in the nucleic acid sequence left after the tagmentation event may be filled using an extending step. In general, an extending step is followed by a ligating step. Extending and/or ligating are performed using appropriate conditions. In some embodiments, the buffer used is an extension-ligation mix buffer (e.g., extension-ligation mix buffer 3, ELM3). A polymerase such as T4 DNA pol Exo- (New England BioLabs, Catalog #M0203S) or Ttaq608 may be used in said extending and/or ligating step. Taq polymerase, or mutants, analogues, or derivatives of any of the aforementioned polymerases may also be used in this step instead.

In some embodiments, double-stranded target nucleic acid fragments are extended. In some embodiments, a second strand of the double-stranded target nucleic acid fragments is extended.

In some embodiments, the 3′ end of the double-stranded target nucleic acid fragments is extended to the 5′ end of a transposon.

In some embodiments, the extending step comprises extending from the 3′ end of a second strand of double-stranded target nucleic acid fragments to the 5′ end of a hairpin UMI.

In some embodiments, the extending step is performed with a strand displacement extension reaction, such as one comprising a Bst DNA polymerase and dNTP mix.

In some embodiments, the extending step is followed by ligation. In these embodiments, a method may comprise treating a polymerase and a ligase to extend and ligate the nucleic acid strands to produce fully double-stranded tagged fragments.

In some embodiments, the extending step comprises extending 9 bases.

In some embodiments, the extending step comprises extending from the 3′ end of the second strand of double-stranded target nucleic acid fragments to the 5′ end of a splint ligation adapter.

In some embodiments, the extending step comprises extending from the 3′ end of the second strand of double-stranded target nucleic acid fragments to a junction in the template switch oligonucleotide by copying the first strand of the double-stranded target nucleic acid fragments.

In some embodiments, there are no gaps in the nucleic acid sequence left after the transposition event. In these embodiments, a method comprises a using a ligase to ligate transposons or polynucleotides with double-stranded target nucleic acid fragment and an extending step is not used.

A wide variety of library preparation methods comprising a step of adapter ligation are known in the art, such as TruSeq and TruSight Oncology 500 (See, e.g., TruSeq® RNA Sample Preparation v2 Guide, 15026495 Rev. F, Illumina, 2014). Exemplary ligated forked adapters are discussed in WO 2007/052006, US Patent Pub. No. 2020/0080145, U.S. Pat. No. 9,868,982, and WO 2020/144373, which are incorporated by reference in their entireties herein. Adapters used with other ligation methods may be used in the present method (See, e.g., Illumina Adapter Sequences, Illumina, 2021). In particular, adapter ligation may allow for more flexible incorporation of adapters (such as adapters with longer lengths) as compared to methods of tagging fragments via tagmentation (wherein adapter sequences are incorporated into fragments during the transposition reaction). In some methods involving tagmentation, additional adapter sequences may be incorporated by PCR reactions, and the present methods may obviate the need for an additional PCR step to incorporate additional adapter sequences.

Ligation technology is commonly used to prepare NGS libraries for sequencing. In some embodiments, the ligation step uses an enzyme to connect specialized adapters to both ends of DNA fragments. In some embodiments, an A-base is added to blunt ends of each strand, preparing them for ligation to the sequencing adapters. In some embodiments, each adapter contains a T-base overhang, providing a complementary overhang for ligating the adapter to the A-tailed fragmented DNA.

Adapter ligation protocols are known to have advantages over other methods. For example, adapter ligation can be used to generate the full complement of sequencing primer hybridization sites for single, paired-end, and indexed reads. In some embodiments, adapter ligation eliminates a need for additional PCR steps to add the index tag and index primer sites.

In some embodiments, the ligating step comprises ligating the 3′ end of the double-stranded target nucleic acid fragments with the 5′ end of a transposon.

In some embodiments, the ligating step comprises ligating the 3′ end of double-stranded target nucleic acid fragments with the 5′ end of transposons.

In some embodiments, the ligating step comprises ligating the 3′ end of the second strand of the double-stranded target nucleic acid fragments with the 5′ end of the universal hybridization tail.

In some embodiments, the ligating step comprises ligating the 3′ end of the second strand of extended double-stranded target nucleic acid fragments with the 5′ end of a first strand of a splint ligation adapter.

E. Template Switching

In some embodiments, a template switch or strand exchange step may be performed after the nucleic acid fragments are released from the transposome complexes. In some embodiments, this template switching step is followed by gap-filling and ligation. In some embodiments, the method can be performed in-tube or in-flowcell.

Template switching refers to the ability of a polymerase to discontinue extending while still binding the newly synthesized strand and to reinitiate synthesis at another nucleic acid strand. In some embodiments, the steps of (1) extending, (2) template switching and (3) re-initiation of synthesis after tagmentation are performed by a polymerase capable of DNA template-switching. In some embodiments, the polymerase is a Moloney murine leukemia virus (MMLV) reverse transcriptase.

In some embodiments, templates are switched from the first strand double-stranded target nucleic acid fragments to an unpaired region of a 3′ template switch oligonucleotide. In some embodiments, a copying step follows the template switching step to copy the unpaired region of the 3′ switch oligonucleotide from the junction in the template switch oligonucleotide to the 5′ end said unpaired region.

F. Amplification

A UMI library can optionally be amplified according to any suitable amplification methodology known in the art and sequenced with one or more sequencing primers. In some embodiments, the UMI library is amplified on a solid support. In some embodiments, the solid support is the same solid support upon which the BLT tagmentation occurs. In such embodiments, the methods and compositions provided herein allow sample preparation to proceed on the same solid support from the initial sample introduction step through amplification and optionally through a sequencing step.

For example, in some embodiments, the UMI library is amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays.” The products of solid-phase amplification reactions such as those described in U.S. Pat. No. 7,985,565 and U.S. Pat. No. 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from UMI library produced according to the methods provided herein. For example, one or more clusters or colonies can be formed via solid-phase PCR whether one or both primers of each pair of amplification primers are immobilized.

In other embodiments, the UMI library is amplified in solution. For example, in some embodiments, the nucleic acid fragments are cleaved or otherwise liberated from the solid support and amplification primers are then hybridized in solution to the liberated molecules. In other embodiments, amplification primers are hybridized to the nucleic acid fragments for one or more initial amplification steps, followed by subsequent amplification steps in solution. Thus, in some embodiments an immobilized nucleic acid template can be used to produce solution-phase amplicons.

It will be appreciated that any of the amplification methodologies described herein or generally known in the art can be utilized with universal or target-specific primers to amplify the UMI library. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify the UMI library. In some embodiments, primers directed specifically to the nucleic acid of interest are included in the amplification reaction.

Other suitable methods for amplification of nucleic acids can include oligonucleotide extension and ligation, rolling circle amplification (RCA) (Lizardi et al., Nat. Genet. 19:225-232 (1998), which is incorporated herein by reference) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference) technologies. It will be appreciated that these amplification methodologies can be designed to amplify the UMI library. For example, in some embodiments, the amplification method can include ligation probe amplification or oligonucleotide ligation assay (OLA) reactions that contain primers directed specifically to the nucleic acid of interest. In some embodiments, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, CA) as exemplified by U.S. Pat. Nos. 7,582,420 and 7,611,869, each of which is incorporated herein by reference in its entirety.

Exemplary isothermal amplification methods that can be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587, each of which is incorporated herein by reference in its entirety. Other non-PCR-based methods that can be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in, for example Lage et al., Genome Research 13:294-307 (2003), each of which is incorporated herein by reference in its entirety. Isothermal amplification methods can be used with the strand-displacing Phi 29 polymerase or Bst DNA polymerase large fragment, 5′→3′ exo- for random primer amplification of genomic DNA. The use of these polymerases takes advantage of their high processivity and strand displacing activity. High processivity allows the polymerases to produce fragments that are 10-20 kb in length. As set forth above, smaller fragments can be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another nucleic acid amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993), incorporated herein by reference in its entirety. The first rounds of amplification are carried out to allow a multitude of initiations on heat denatured DNA based on individual hybridization from the randomly synthesized 3′ region. Due to the nature of the 3′ region, the sites of initiation are contemplated to be random throughout the genome. Thereafter, the unbound primers can be removed and further replication can take place using primers complementary to the constant 5′ region.

In some embodiments, the amplifying step comprises adding oligonucleotides to one or both ends of the nucleic acid fragments for attaching the library to a solid support.

In some embodiments, the amplifying step comprises adding at least a first-read sequencing oligonucleotide and/or a second-read sequencing oligonucleotide. In some embodiments, the amplifying step comprises adding at least a P5 oligonucleotide and a P7 oligonucleotide. In some embodiments, the amplifying step comprises adding at least a plurality of i5 oligonucleotides and a plurality of i7 oligonucleotides.

In some embodiments, after the amplifying step, a method may comprise selecting for amplified nucleic acid fragments within a size range after the amplifying step.

G. Methods for Producing UMI Libraries

While adapters may comprise more than one adapter sequence in any combination or order from 5′ to 3′, the present disclosure provides adapters that may be used in a variety of embodiments. The present disclosure also provides multiple methods that may be used with the adapters described herein. The methods of the present disclosure may comprise one or more of the following adapters and methods.

1. Method for Producing a UMI Library Using a Single UMI

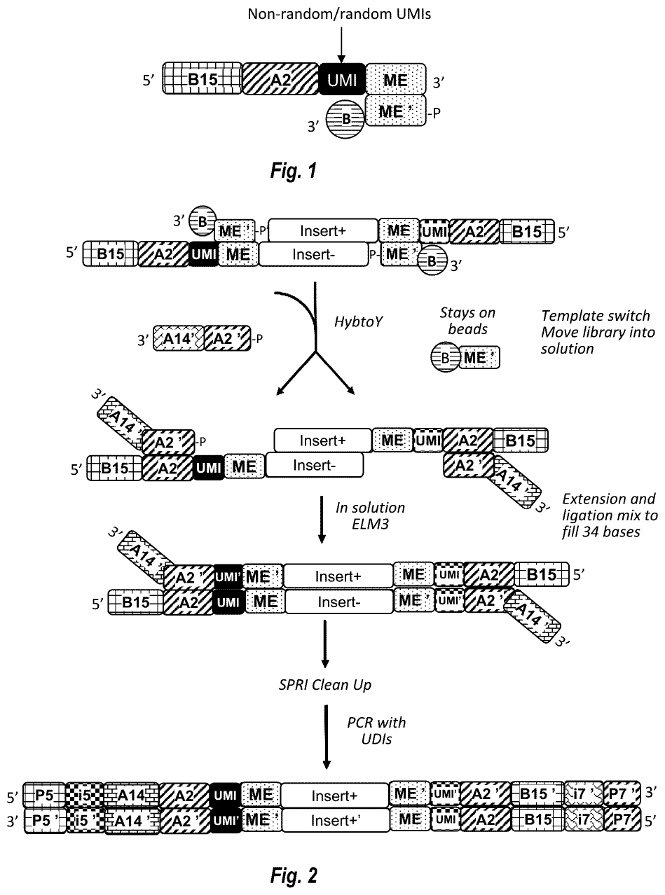

As shown in FIG. 1, an exemplary adapter comprises the following adapter sequences on its first strand from 5′ to 3′: B15, A2, UMI, and ME. In the adapter, the UMI is located between A2 and ME. The UMIs may comprise nrUMIs and/or rUMIs. On its second strand, the adapter comprises a sequence that is complementary to ME. The adapter also comprises a biotin tag so that the adapter may be used with a solid support. In other embodiments, a solid support is not used and an investigator may employ solution-phase transposome complexes.

As shown in FIG. 2 and described in Example 1, an exemplary method of producing a UMI library comprises (1) producing a double-stranded nucleic acid library wherein each fragment in the library comprises a UMI, wherein the method comprises: (a) applying a sample comprising double-stranded target nucleic acids to a first transposome complex comprising: (i) a first transposase, (ii) a first transposon comprising a first 3′ end transposon end sequence, a first adapter sequence, and a first UMI, and (iii) a second transposon comprising a sequence all or partially complementary to the first 3′ end transposon end sequence; (2) tagmenting the double-stranded target nucleic acids with the first and second transposons to produce double-stranded target nucleic acid fragments comprising the first adapter sequence and the first UMI, (3) releasing the double-stranded target nucleic acid fragments from the first transposome complex, (4) optionally extending the double-stranded target nucleic acid fragments, thereby copying the single UMI to produce a duplex UMI, (5) ligating the transposon or extended transposons with the double-stranded target nucleic acid fragments, (6) producing double-stranded target nucleic acid fragments comprising the UMIs, and (7) amplifying the double-stranded target nucleic acid fragments.

In this exemplary method, the first UMI in the first transposon is located between the first adapter sequence and the first 3′ transposon end sequence.

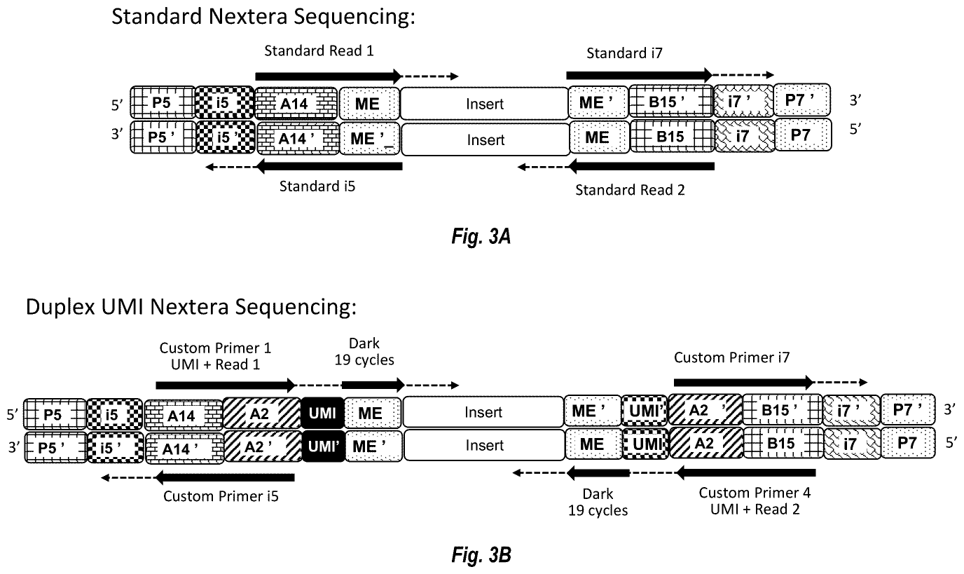

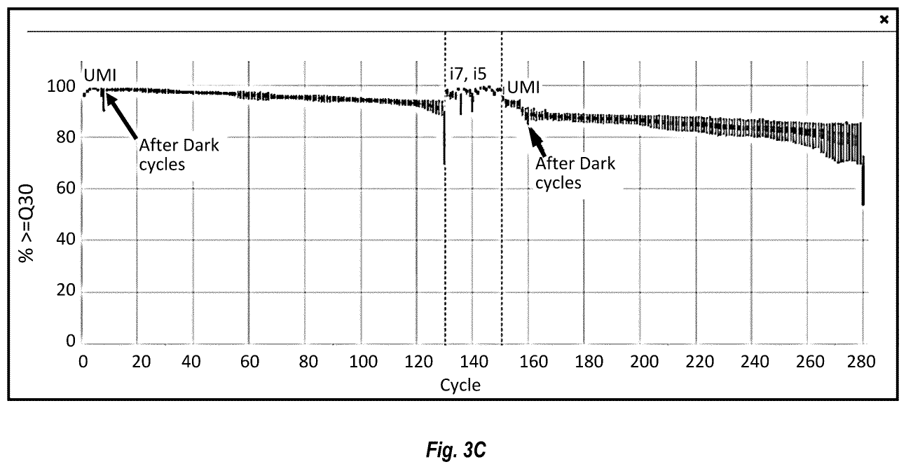

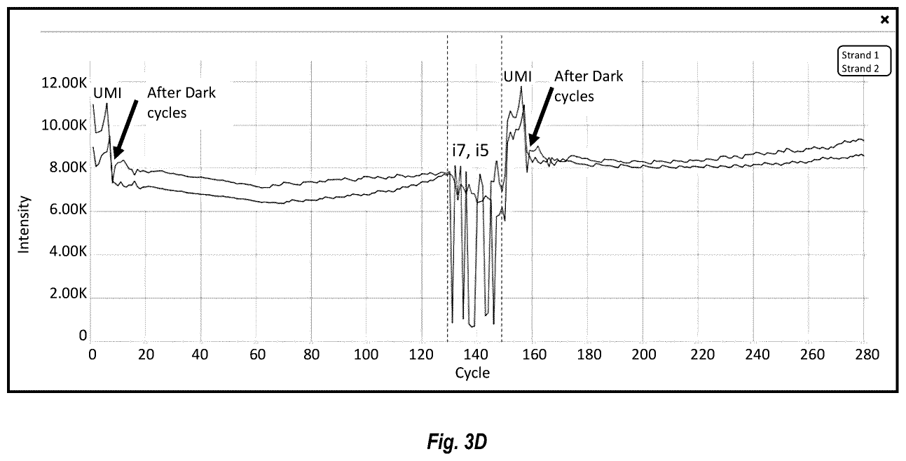

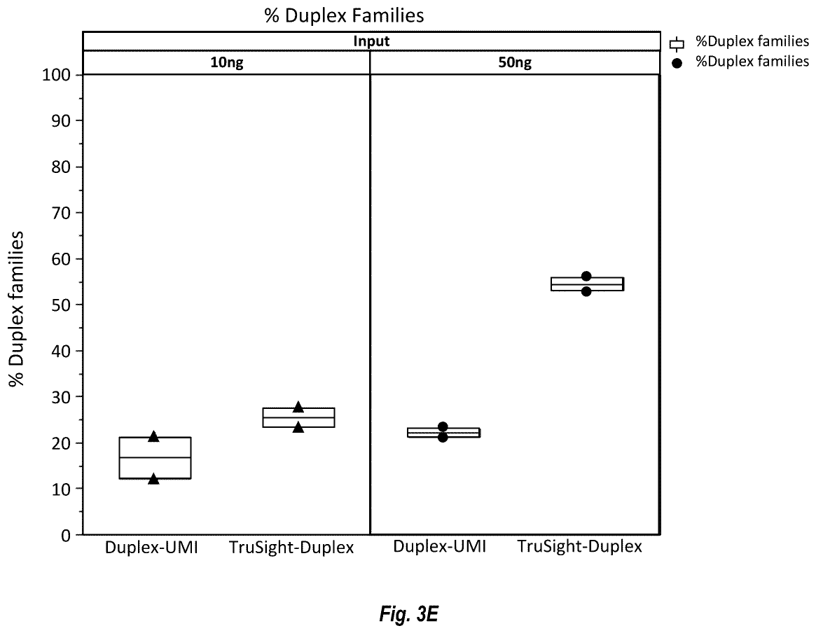

As shown in FIG. 3B and described in Example 2, an exemplary method of sequencing a UMI library comprises 19 dark cycles (discussed in Section III.A below). In this method, the 19 bases of the ME sequence are not imaged during the 19 dark cycles. This method uses the following four primers: Custom Primer 1 UMI+Read 1, Custom Primer i5, Custom Primer i7, and Custom Primer 4 UMI+Read 2.

Using this exemplary adapter and method, a UMI library is produced wherein the first UMI is on a first strand of the double-stranded target nucleic acid fragments, the second UMI is on the second strand of the double-stranded target nucleic acid fragments.

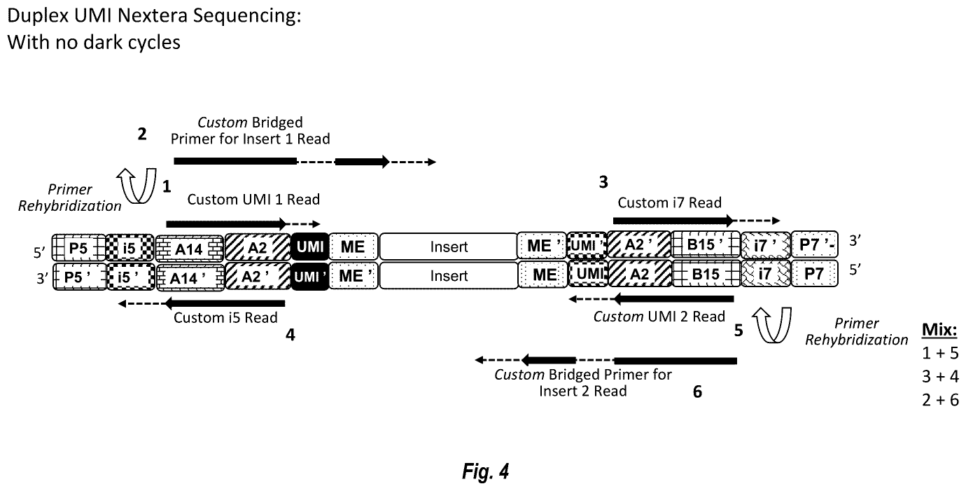

An alternative exemplary method of sequencing a UMI library may be used. As shown in FIG. 4 and described in Example 3, the exemplary method comprises the following 6 custom primers: Custom UMI 1 Read (SEQ ID NO: 8), Custom Bridged Primer for Insert 1 Read (SEQ ID NO: 9), Custom i7 Read (SEQ ID NO: 10), Custom i5 Read (SEQ ID NO: 11), Custom UMI 2 Read (SEQ ID NO: 12), and Custom Bridged Primer for Insert 2 Read (SEQ ID NO: 13). In this sequencing method, primers with SEQ ID NOS: 1 and 5 are combined, primers with SEQ ID NOS: 3 and 4 are combined, and primers with SEQ ID NOS: 2 and 6 are combined.

2. Method for Producing a UMI Library with a UMI-BLT

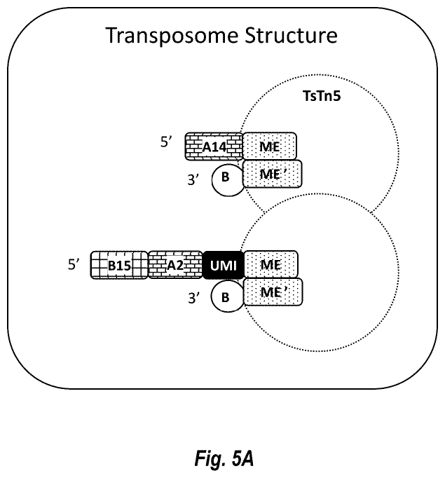

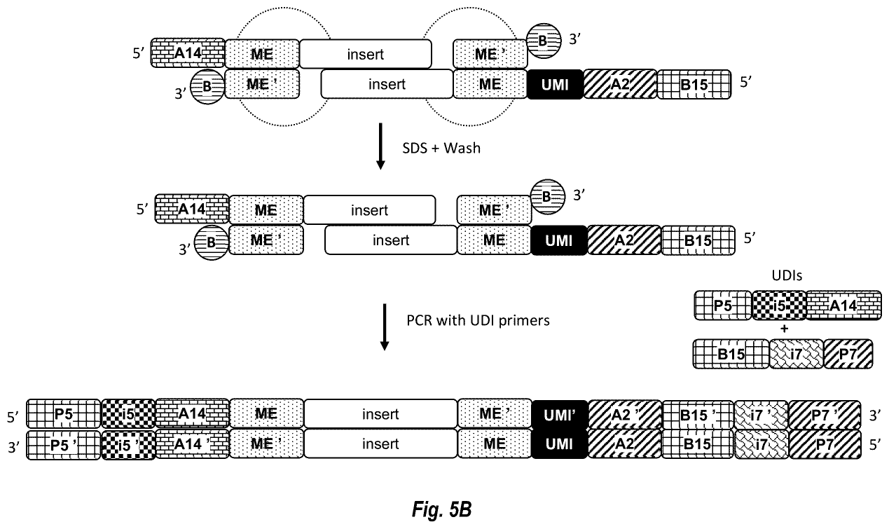

Two exemplary adapters are shown in FIG. 5A. The first adapter comprises the following sequences on its first strand from 5′ to 3′: A15 and ME. The first adapter also comprises a sequence complementary to ME on its second strand.

The second adapter comprises the following sequences on its first strand from 5′ to 3′: B15, A2, UMI, and ME. The UMI is located between A2 and ME. The second adapter also comprises a sequence complementary to ME on its second strand. The first and second adapters comprise a biotin tag.

As shown in FIG. 5B and described in Example 4, an exemplary method of producing a UMI library comprises (1) producing a double-stranded nucleic acid library wherein each fragment in the library comprises a UMI, wherein the method comprises: (a) applying a sample comprising double-stranded target nucleic acids to a first transposome complex comprising: (i) a first transposase, (ii) a first transposon comprising a first 3′ end transposon end sequence, a first adapter sequence, and a first UMI, and (iii) a second transposon comprising a sequence all or partially complementary to the first 3′ end transposon end sequence; (2) tagmenting the double-stranded target nucleic acids with the first and second transposons to produce double-stranded target nucleic acid fragments comprising the first adapter sequence and the first UMI, (3) releasing the double-stranded target nucleic acid fragments from the first transposome complex, (4) optionally extending the double-stranded target nucleic acid fragments, (5) producing double-stranded target nucleic acid fragments comprising the UMIs, and (7) amplifying the double-stranded target nucleic acid fragments.

In this exemplary method, the first UMI in the first transposon is located between the first adapter sequence and the first 3′ transposon end sequence.

This exemplary method further comprises a second transposome complex comprising (1) a second transposase, (2) a third transposon comprising a second adapter sequence and a second 3′ transposon end sequence, and (3) a fourth transposon comprising a sequence all or partially complementary to the second 3′ end transposon end sequence.

Using the exemplary adapters and method described herein, a UMI library is produced wherein the first UMI is on the first strand of the double-stranded target nucleic acid fragments.

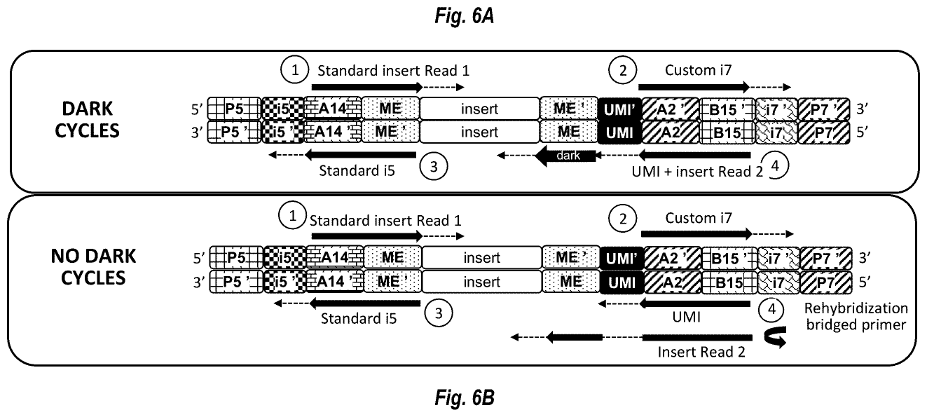

As shown in FIG. 6A and described in Example 5, an exemplary method of sequencing a UMI library comprises dark cycles and the following four primers: Standard Insert Read 1, Custom i7, Standard i5, and UMI+Insert Read 2.

An alternative exemplary method of sequencing a UMI library may be used. As shown in FIG. 6B and described in Example 6, the exemplary method comprises the following four primers: Standard Insert Read 1, Custom i7, Standard i5, UMI primer, and Insert Read 2 Bridged Primer. In the method, a bridged primer rehybridization step is used where the UMI primer is displaced by the Insert Read 2 Bridged Primer.

3. Method for Producing a UMI Library Prepared from Cell-Free DNA (cfDNA)

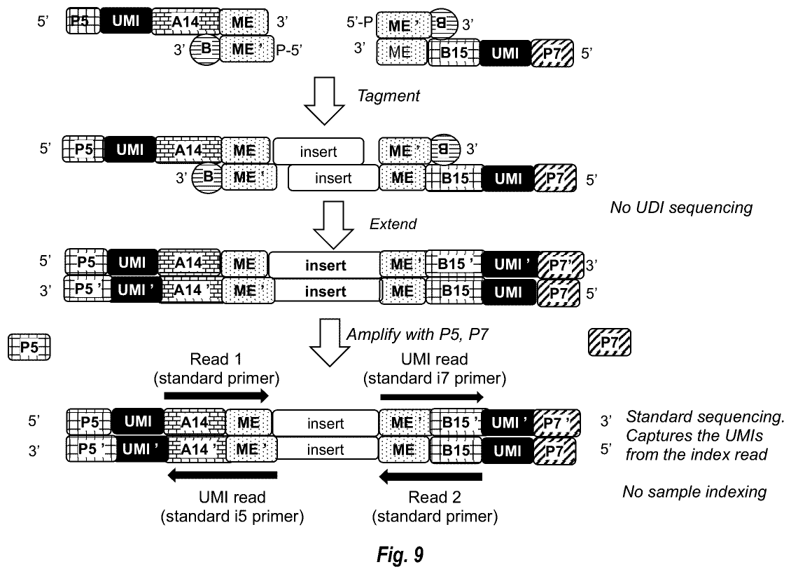

Two exemplary adapters are shown in FIG. 9. The first adapter comprises the following sequences on its first strand from 5′ to 3′: P5, UMI, A14, and ME. The first adapter also comprises a sequence complementary to ME on its second strand. The UMI is located between P5 and A14.

The second adapter comprises the following sequences on its first strand from 5′ to 3′: P7, UMI, B15, and ME. The UMI is located between P7 and B15. The second adapter also comprises a sequence complementary to ME on its second strand. The first and second adapters comprise a biotin tag.

As shown in FIG. 9 and described in Example 7, an exemplary method of producing a UMI library comprises (1) producing a double-stranded nucleic acid library wherein each fragment in the library comprises a UMI, wherein the method comprises: (a) applying a sample comprising double-stranded target nucleic acids to a first transposome complex comprising: (i) a first transposase, (ii) a first transposon comprising a first 3′ end transposon end sequence, a first adapter sequence, and a first UMI, and (iii) a second transposon comprising a sequence all or partially complementary to the first 3′ end transposon end sequence; (2) tagmenting the double-stranded target nucleic acids with the first and second transposons to produce double-stranded target nucleic acid fragments comprising the first adapter sequence and the first UMI, (3) releasing the double-stranded target nucleic acid fragments from the first transposome complex, (4) optionally extending the double-stranded target nucleic acid fragments, (5) producing double-stranded target nucleic acid fragments comprising the UMIs, and (7) amplifying the double-stranded target nucleic acid fragments. The first adapter sequence in the first transposon is located between the first UMI and the first 3′ transposon end sequence.

This exemplary method further comprises a second transposome complex comprising (1) a second transposase, (2) a third transposon comprising a second adapter sequence and a second 3′ transposon end sequence, and (3) a fourth transposon comprising a sequence all or partially complementary to the second 3′ end transposon end sequence.

This method further comprises (1) the third transposon further comprises a second UMI, and (2) the second adapter sequence is located between the second UMI and the second 3′ transposon end sequence. In this method, the tagmenting step produces double-stranded target nucleic acid fragments comprising: (1) a first strand comprising the first adapter sequence and the first UMI, and (2) a second strand comprising the second adapter sequence and the second UMI.

Using the exemplary adapters and method described herein, a UMI library is produced wherein a first copy of the first UMI is on the first strand and a second copy of the first UMI is on the second strand of the double-stranded target nucleic acid fragments.

As shown in FIG. 9 and described in Example 8, an exemplary method of sequencing a UMI library comprises the following four primers: Read 1 (standard primer), UMI read (standard i7 primer), UMI read (standard i5 primer) and Read 2 (standard primer).

An alternative exemplary method of sequencing a UMI library may be used. As shown in FIG. 6B and described in Example 6, the exemplary method comprises the following four primers: Standard Insert Read 1, Custom i7, Standard i5, UMI primer, and Insert Read 2 Bridged Primer. In the method, a bridged primer rehybridization step is used where the UMI primer is displaced by the Insert Read 2 Bridged Primer.

4. A First Method for Producing a UMI Library with UDIs and Duplex UMI

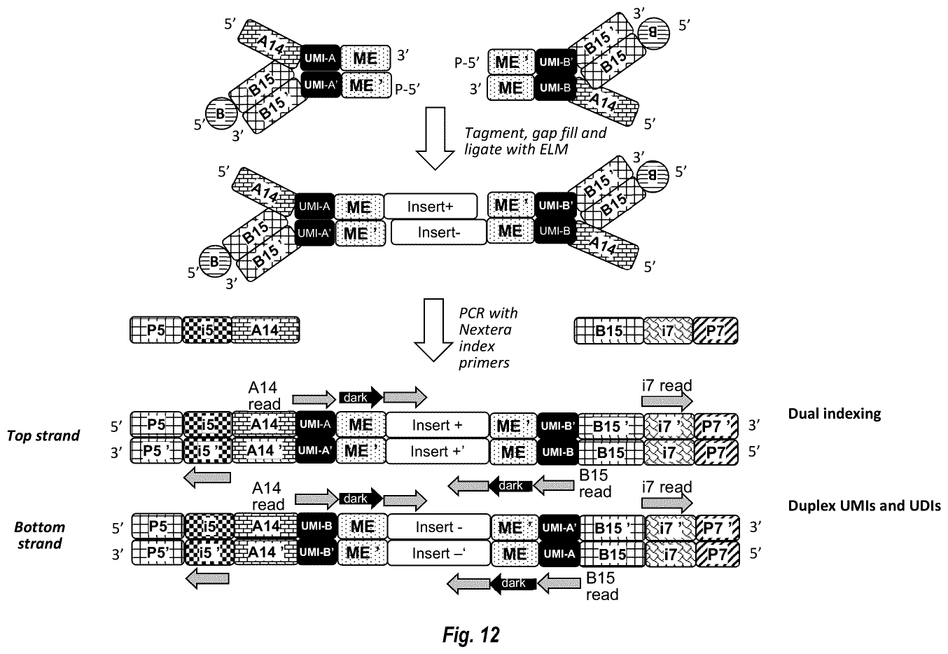

Two exemplary adapters are shown in FIG. 12. The first and second adapters are forked adapters.

The first adapter comprises the following sequences on its first strand from 5′ to 3′: A14, UMI-A, and ME. The first adapter also comprises the following sequence on its second strand from 5′ to 3′: ME′, UMI-A′, and a B15 duplex wherein B15 is hybridized to B15′. UMI-A is located between A14 and ME. UMI-A′ is located between ME′ and the B15 duplex.

The second adapter comprises the following sequences on its first strand from 5′ to 3′: A14, UMI-B, and ME. The second adapter also comprises the following sequence on its second strand from 5′ to 3′: ME′, UMI-B′, and B15 duplex. UMI-B is located between A14 and ME.

The first and second adapters each comprise a biotin tag.

As shown in FIG. 12 and described in Example 9, an exemplary method of producing a UMI library comprises (1) applying a sample comprising double-stranded target nucleic acids to a first transposome complex and a second transposome complex, (2) tagmenting the double-stranded target nucleic acids with the forked adapter transposons to produce double-stranded target nucleic acid fragments comprising the first and second copies of the first adapter sequences, the first UMI, the first and second copies of the second adapter sequences, and the second UMI, (3) releasing the double-stranded target nucleic acid fragments from the transposome complexes, (4) optionally extending the double-stranded target nucleic acid fragments, (5) ligating the forked adapter transposons or the extended forked adapter transposons with the double-stranded target nucleic acid fragments, (6) producing double-stranded target nucleic acid fragments comprising the UMIs, and (7) amplifying the double-stranded target nucleic acid fragments.

In this method, the first transposome complex comprises (1) a first transposase and (2) a first forked adapter transposon on a first strand of the double-stranded target nucleic acid fragments, wherein (i) the first strand of the first forked adapter transposon comprises a first 3′ end transposon end sequence, a first copy of a first adapter sequence, and a first UMI, and (ii) the second strand of the first forked adapter transposon comprises a first copy of a second adapter sequence, and a sequence all or partially complementary to the first strand of the first forked adapter transposon.

Further, the second transposome complex comprises (1) a second transposome complex comprising: (i) a second transposase and (ii) a second forked adapter transposon on a second strand of the double-stranded target nucleic acid fragments, wherein (a) the first strand of the second forked adapter transposon comprises a second 3′ end transposon end sequence, a second copy of the first adapter sequence, and a second UMI, and (b) the second strand of the second forked adapter transposon comprises a second copy of the second adapter, and a sequence all or partially complementary to the first strand of the second forked adapter transposon.

As shown in FIG. 6A and described in Example 5, an exemplary method of sequencing a UMI library comprises dark cycles and the following four primers: Standard Insert Read 1, Custom i7, Standard i5, and UMI+Insert Read 2.

An alternative exemplary method of sequencing a UMI library may be used. As shown in FIG. 6B and described in Example 6, the exemplary method comprises the following four primers: Standard Insert Read 1, Custom i7, Standard i5, UMI primer, and Insert Read 2 Bridged Primer. In the method, a bridged primer rehybridization step is used where the UMI primer is displaced by the Insert Read 2 Bridged Primer.

As shown in FIG. 12 and described in Example 10, an exemplary method of sequencing a UMI library comprises dark cycles and the following primers: A14 Read, B15 Read, i7 Read, and i5 Read.

5. A Second Method for Producing a UMI Library with UDIs and Duplex UMI

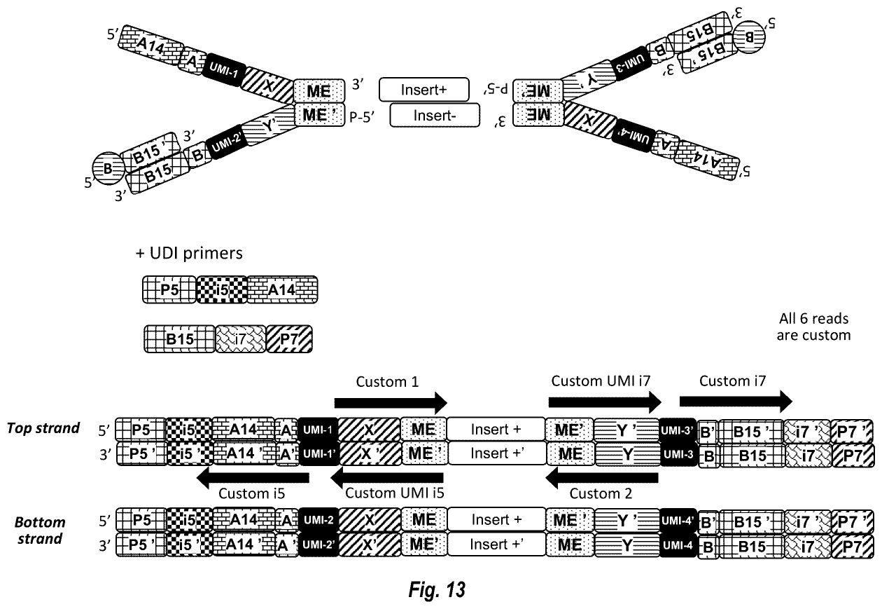

Two exemplary adapters are shown in FIG. 13. The first and second adapters are forked adapters. In order to use duplex sequencing with this method of producing a UMI library, the annealed pair of UMIs within each forked adapter are not complementary. (See FIG. 12 for comparison.)

Each adapter in this method is double stranded and contains two UMIs, with one UMI on each strand (FIG. 13). The two strands are annealed at the ME region to produce a forked adapter with noncomplementary, duplex UMI. Because the duplex UMIs do not contain complementary sequences, each adapter is annealed separately from the other.

The first adapter comprises the following sequences on its first strand from 5′ to 3′: A14, A, UMI-1, X, and ME. The first adapter also comprises the following sequence on its second strand from 5′ to 3′: ME′, Y, UMI-2′, B, and a B15 duplex wherein B15 is hybridized to B15′. UMI-1 is located between A and UMI-1. UMI-2′ is located between ME′ and B.

The second adapter comprises the following sequences on its first strand from 5′ to 3′: A14, A, UMI-4′, X, and ME. The second adapter also comprises the following sequence on its second strand from 5′ to 3′: ME′, Y′, UMI-3, B, and a B15 duplex. UMI-4′ is located between A and X. UMI-3 is located between B and Y′.

The first and second adapters each comprise a biotin tag.

As shown in FIG. 13 and described in Example 11, an exemplary method of producing a UMI library comprises (1) applying a sample comprising double-stranded target nucleic acids to a first transposome complex and a second transposome complex, (2) tagmenting the double-stranded target nucleic acids with the forked adapter transposons to produce double-stranded target nucleic acid fragments comprising the first and second copies of the first adapter sequences, the first UMI, the first and second copies of the second adapter sequences, and the second UMI, (3) releasing the double-stranded target nucleic acid fragments from the transposome complexes, (4) optionally extending the double-stranded target nucleic acid fragments, (5) ligating the forked adapter transposons or the extended forked adapter transposons with the double-stranded target nucleic acid fragments, (6) producing double-stranded target nucleic acid fragments comprising the UMIs, and (7) amplifying the double-stranded target nucleic acid fragments.

In this method, the first transposome complex comprises (1) a first transposase and (2) a first forked adapter transposon on a first strand of the double-stranded target nucleic acid fragments, wherein (i) the first strand of the first forked adapter transposon comprises a first 3′ end transposon end sequence, a first copy of a first adapter sequence, and a first UMI, and (ii) the second strand of the first forked adapter transposon comprises a first copy of a second adapter sequence, and a sequence all or partially complementary to the first strand of the first forked adapter transposon.

Further, the second transposome complex comprises (1) a second transposome complex comprising: (i) a second transposase and (ii) a second forked adapter transposon on a second strand of the double-stranded target nucleic acid fragments, wherein (a) the first strand of the second forked adapter transposon comprises a second 3′ end transposon end sequence, a second copy of the first adapter sequence, and a second UMI, and (b) the second strand of the second forked adapter transposon comprises a second copy of the second adapter, and a sequence all or partially complementary to the first strand of the second forked adapter transposon.

Further, (1) the first strand of the first forked adapter transposon further comprises a third adapter sequence, (2) the second strand of the first forked adapter transposon further comprises a fourth adapter sequence and a third UMI, and (3) the first strand of the second forked adapter transposon further comprises a sequence all or partially complementary to the third adapter sequence, (4) the second strand of the second forked adapter transposon further comprises a sequence all or partially complementary to the fourth adapter sequence and a fourth UMI, and (5) the tagmenting step produces double-stranded target nucleic acid fragments further comprising the third UMI and the fourth UMI.

As shown in FIG. 13 and described in Example 12, an exemplary method of sequencing a UMI library comprises dark cycles and the following 6 custom primers: Custom 1, Custom UMI i7, Custom i7, Custom 2, Custom UMI i5, and Custom i5.

6. A Method for Producing In-Line UMIs Using an Adapter Comprising a Hairpin UMI and a Universal Hybridizing Tail

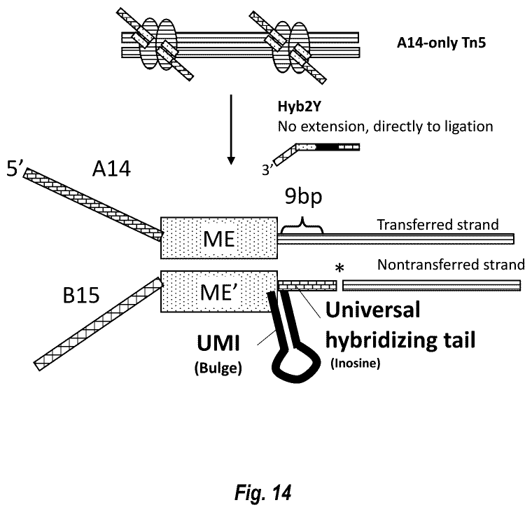

An exemplary 3′ adapter is shown in FIG. 14 and described in Example 13. The adapter comprises following from 5′ to 3′: universal hybridizing tail, hairpin UMI, ME′, and B15. The hairpin UMI comprises a 3 or 4 base pair stem structure that forms a bulge. The universal hybridizing tail comprises inosines that can bind to any DNA molecule, which allows for hybridization to the exposed 5′ bases of the transferred strand.

As described in Example 13, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double-stranded target nucleic acid fragments from the transposome complex, (4) hybridizing a polynucleotide comprising a second adapter sequence, a UMI, and a sequence all or partially complementary to the first 3′ end transposon sequence, (5) ligating the polynucleotide with the double-stranded target nucleic acid fragments, (6) producing double-stranded target nucleic acid fragments comprising the UMI, wherein the UMI is located directly adjacent to the 3′ end of the insert DNA, and (7) amplifying the double-stranded target nucleic acid fragments.

Further, the ligating step comprises ligating the 3′ end of the second strand of the double-stranded target nucleic acid fragments with the 5′ end of the universal hybridization tail.

Further, the hairpin UMI is stable during the extending step and/or the ligating step, but not during the amplifying step.

According to this method, the UMI is on the first strand of the double-stranded target nucleic acid fragments.

The exemplary adapter and method described herein produces a UMI library wherein the in-line UMI is adjacent to the 3′ end of the insert DNA (FIG. 20). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

7. Δ Method for Producing In-Line UMIs Comprising a Hairpin UMI

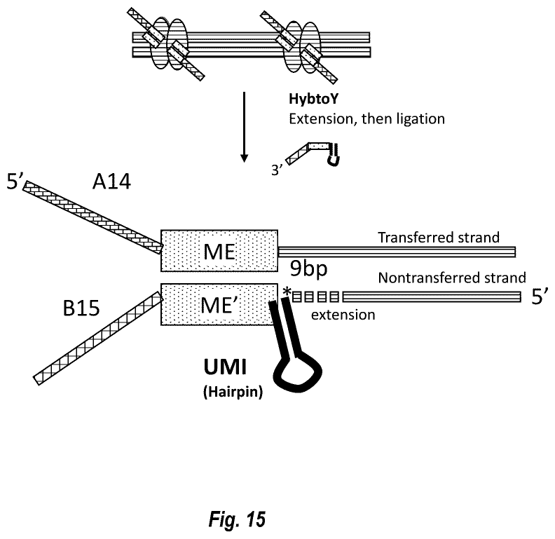

An exemplary 3′ adapter is shown in FIG. 15 and described in Example 14. The adapter is a polynucleotide comprising the following from 5′ to 3′: hairpin UMI, ME′, and B15. The hairpin UMI comprises a 3 or 4 base pair stem structure that forms a bulge.

As described in Example 14, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double-stranded target nucleic acid fragments from the transposome complex, (4) hybridizing a polynucleotide comprising a second adapter sequence, a UMI, and a sequence all or partially complementary to the first 3′ end transposon sequence, (5) extending a second strand of the double-stranded target nucleic acid fragments, (6) ligating the extended polynucleotide with the double-stranded target nucleic acid fragments, (7) producing double-stranded target nucleic acid fragments comprising the UMI, wherein the UMI is located directly adjacent to the 3′ end of the insert DNA, and (8) amplifying the double-stranded target nucleic acid fragments.

Further, the extending step comprises extending from a 3′ end of the second strand of the double-stranded target nucleic acid fragments to the 5′ end of the hairpin UMI.

Further, the ligating step comprises ligating the 3′ end of the second strand of the double-stranded target nucleic acid fragments with the 5′ end of the hairpin UMI.

Further, the hairpin UMI is stable during the extending step and/or the ligating step, but not during the amplifying step.

According to this method, the UMI is on the first strand of the double-stranded target nucleic acid fragments.

The exemplary adapter and method described herein produces a UMI library wherein the UMI is adjacent to the 3′ end of the insert DNA (FIG. 20). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

8. A First Method for Producing In-Line UMIs Comprising a Splint Ligation Adapter

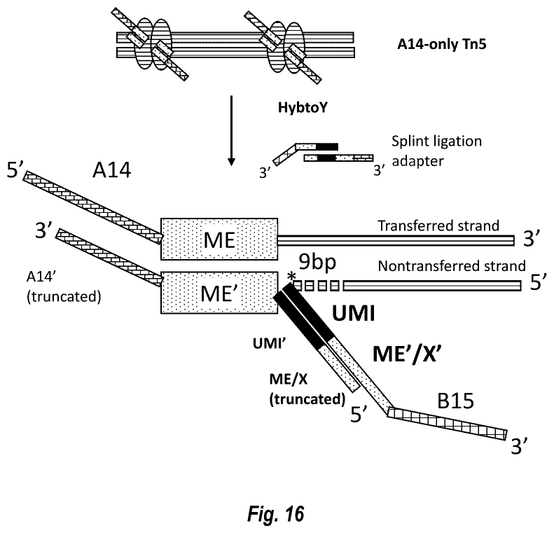

An exemplary 3′ adapter is shown in FIG. 16 and described in Example 15a. The adapter is a polynucleotide comprising 3′ splint ligation adapter complex comprising a partially double-stranded. The two portions of the adapter are the splint (see FIG. 16, 3′ splint ligation adapter, bottom strand), and the tail (see FIG. 16, 3′ splint ligation adapter, top strand). The splint portion contains the following from 5′ to 3′: ME, UMI′, ME′, truncated A14′. The tail portion comprises the following from 5′ to 3′: UMI, ME′ and B15. The complex is formed via hybridization of UMI and ME sequences.

As described in Example 15a, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double-stranded target nucleic acid fragments from the transposome complex, (4) hybridizing a polynucleotide comprising a second adapter sequence, a UMI, and a sequence all or partially complementary to the first 3′ end transposon sequence, (5) ligating the polynucleotide with the double-stranded target nucleic acid fragments, (6) producing double-stranded target nucleic acid fragments comprising the UMI, wherein the UMI is located directly adjacent to the 3′ end of the insert DNA, and (7) amplifying the double-stranded target nucleic acid fragments.

Further, the extending step comprises extending 9 bases from a 3′ end of the second strand of the double-stranded target nucleic acid fragments to the 5′ end of the splint ligation adapter.

Further, the ligating step comprises ligating the 3′ end of the second strand of the extended double-stranded target nucleic acid fragments with the 5′ end of a first strand of the splint ligation adapter.

According to this method, the UMI is on the first strand of the double-stranded target nucleic acid fragments.

The exemplary adapter and method described herein produces a UMI library wherein the UMI is adjacent to the 3′ end of the insert DNA (FIG. 20). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

9. A Second Method for Producing In-Line UMIs Comprising a Splint Ligation Adapter

An exemplary 3′ adapter is shown in FIG. 16 and described in Example 15b. The adapter is a polynucleotide comprising a 3′ splint ligation adapter complex comprising a partially double-stranded. The two portions of the adapter are the splint (see FIG. 16, 3′ splint ligation adapter, bottom strand), and the tail (see FIG. 16, 3′ splint ligation adapter, top strand). The splint portion contains the following from 5′ to 3′: X, UMI′, ME′, truncated A14′, wherein X is a 3′ TruSeq™ adapter sequence which may be full-length or truncated. The tail portion comprises the following from 5′ to 3′: UMI, X′ and B15. The complex is formed via hybridization of UMI and X sequences.

As described in Example 15b, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double-stranded target nucleic acid fragments from the transposome complex, (4) hybridizing a polynucleotide comprising a second adapter sequence, a UMI, and a sequence all or partially complementary to the first 3′ end transposon sequence, (5) ligating the polynucleotide with the double-stranded target nucleic acid fragments, (6) producing double-stranded target nucleic acid fragments comprising the UMI, wherein the UMI is located directly adjacent to the 3′ end of the insert DNA, and (7) amplifying the double-stranded target nucleic acid fragments.

Further, the extending step comprises extending 9 bases from a 3′ end of the second strand of the double-stranded target nucleic acid fragments to the 5′ end of the splint ligation adapter.

Further, the ligating step comprises ligating the 3′ end of the second strand of the extended double-stranded target nucleic acid fragments with the 5′ end of a first strand of the splint ligation adapter.

According to this method, the UMI is on the first strand of the double-stranded target nucleic acid fragments.

The exemplary adapter and method described herein produces a UMI library wherein the UMI is adjacent to the 3′ end of the insert DNA (FIG. 20). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

10. A First Method for Producing In-Line UMIs Comprising a 3′ Template Switch Oligonucleotide

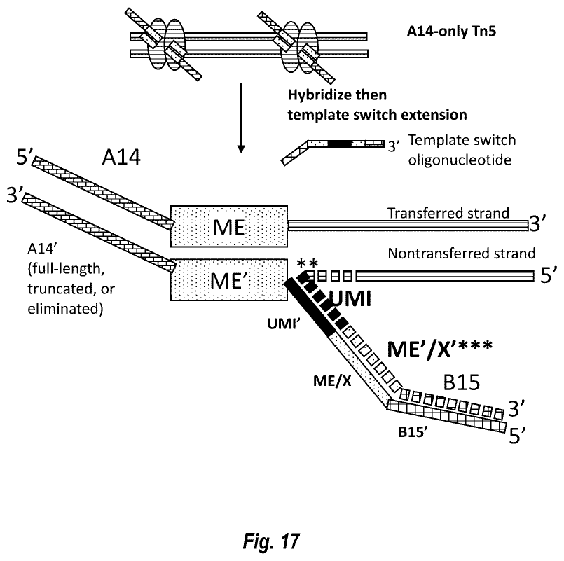

An exemplary 3′ adapter is shown in FIG. 17 and described in Example 16a. The adapter is a polynucleotide comprising a template switch oligonucleotide about 70 nucleotides in length and contains the following from 5′ to 3′: B15′, ME or X, UMI′, ME′, and A14′.

As described in Example 16a, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double-stranded target nucleic acid fragments from the transposome complex, (4) hybridizing a polynucleotide comprising a second adapter sequence, a UMI, and a sequence all or partially complementary to the first 3′ end transposon sequence, (5) ligating the polynucleotide with the double-stranded target nucleic acid fragments, (6) producing double-stranded target nucleic acid fragments comprising the UMI, wherein the UMI is located directly adjacent to the 3′ end of the insert DNA, and (7) amplifying the double-stranded target nucleic acid fragments.

Further, the extending step (1) extending from a 3′ end of the second strand of the double-stranded target nucleic acid fragments to a junction in the template switch oligonucleotide by copying the first strand of the double-stranded target nucleic acid fragments, (2) switching templates from the first strand to an unpaired region of the 3′ template switch oligonucleotide, and (3) copying the unpaired region of the 3′ template switch oligonucleotide from the junction to the 5′ end of the unpaired region of the 3′ template switch oligonucleotide.

According to this method, the UMI is on the first strand of the double-stranded target nucleic acid fragments.

The exemplary adapter and method described herein produces a UMI library wherein the UMI is adjacent to the 3′ end of the insert DNA (FIG. 20). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

11. A Second Method for Producing In-Line UMIs Comprising a Template Switch Oligonucleotide, Wherein the Oligonucleotide Comprises a Modification in A14′

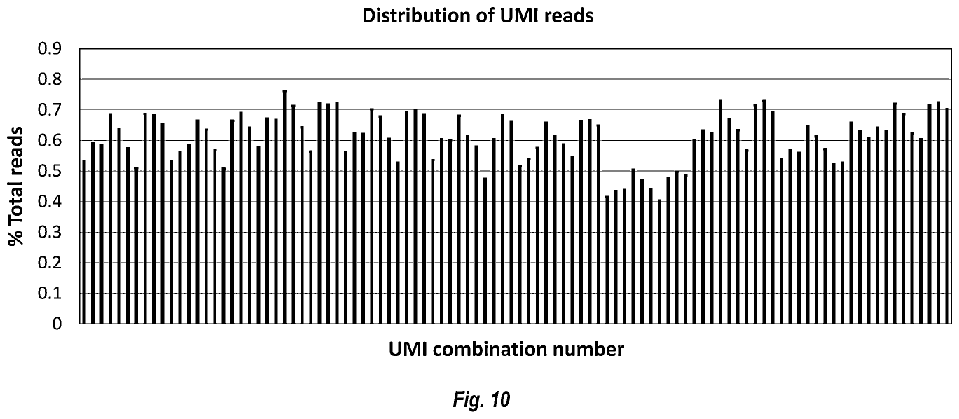

An exemplary 3′ adapter is shown in FIG. 17 and described in Example 16b. The adapter is a polynucleotide comprising a template switch oligonucleotide about 70 nucleotides in length and contains the following from 5′ to 3′: B15′, ME or X, UMI′, ME′, and optionally part of the A14′. The A14′ sequence is truncated or eliminated. Thus, the adapter is the same as the adapter discussed in FIG. 10 above, except the adapter in in FIG. 10 above has the A14′ sequence, whereas in this embodiment the A14′ sequence is truncated or eliminated.

As described in Example 16b, this exemplary method comprises the steps as disclosed in FIG. 10 above.

According to this method, the UMI is on the first strand of the double-stranded target nucleic acid fragments.

The exemplary adapter and method described herein produces a UMI library wherein the UMI is adjacent to the 3′ end of the insert DNA (FIG. 20). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

12. A Method for Producing In-Line UMIs Comprising a 5′ Double-Stranded Adapter, a Polymerase Extension Step and a Proximity Ligation Step

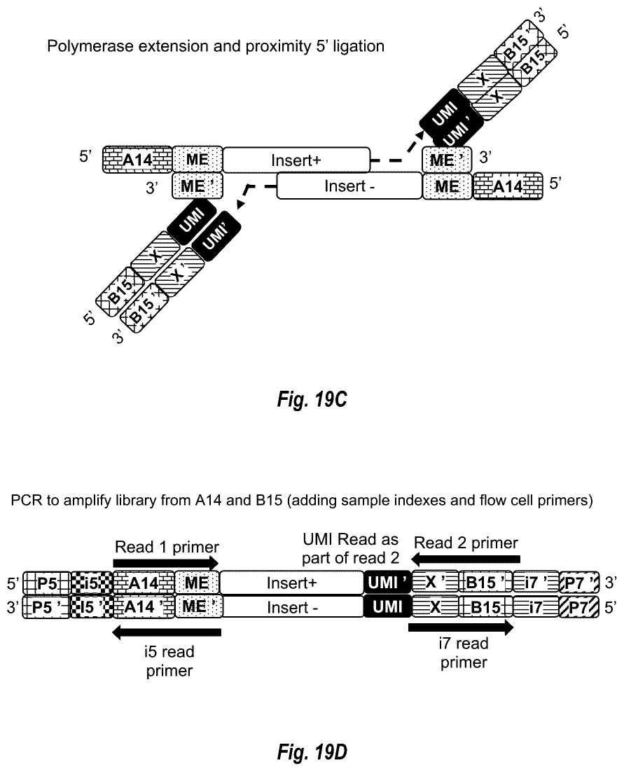

An exemplary adapter is shown in FIG. 19B. The adapter comprises a 5′ double-stranded comprising two oligonucleotides. The first oligonucleotide comprises the following from 5′ to 3′: B15, X, and UMI. The second oligonucleotide comprises the following from 5′ to 3′: UMI′, X′, and B15′. The first and second oligonucleotides are hybridized to form the double-stranded adapter.

As described in Example 16d and shown in FIGS. 19A-C, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double stranded target nucleic acid fragments from transposome complex, (4) hybridizing a first polynucleotide comprising a UMI, and a second adapter sequence, (5) adding a second polynucleotide comprising regions complementary to the first polynucleotide to produce a double-stranded adapter, (6) extending a second strand of the double-stranded target nucleic acid fragments, (7) ligating the double-stranded adapter with the double-stranded target nucleic acid fragments, (8) producing double stranded target nucleic acid fragments comprising UMI, wherein the UMI is located between the double-stranded target nucleic acid fragments and the second adapter sequence, and (9) amplifying the double-stranded target nucleic acid fragments. The ligating step above is termed “proximity ligation” because (as shown in FIG. 19B) the 5′ phosphate and the 3′OH that are being ligated are not hybridized to the same template strand.

The exemplary adapter and method described herein produces a UMI library wherein the UMI is adjacent to the 3′ end of the insert DNA (FIG. 19d). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

13. A Method for Producing In-Line UMIs Comprising a 5′ Single-Stranded Polymerase Template Switch Oligonucleotide

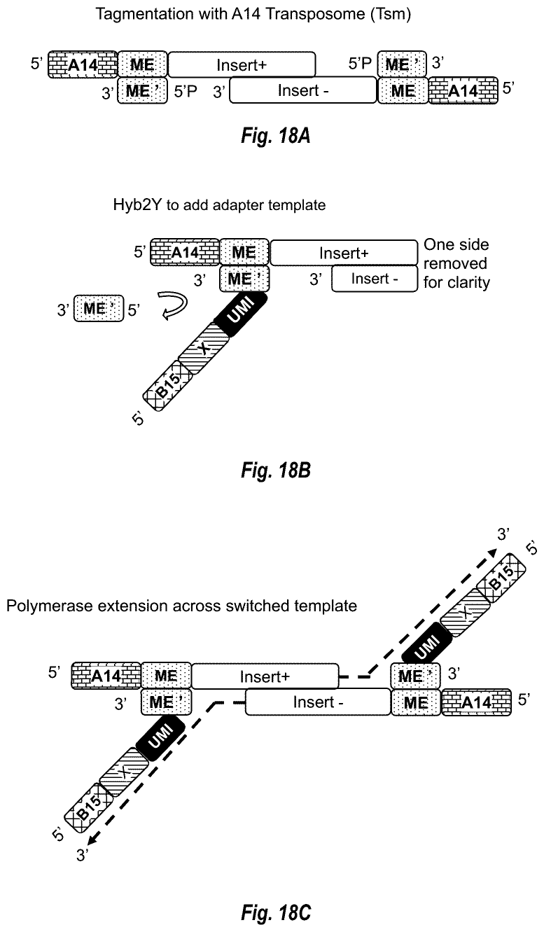

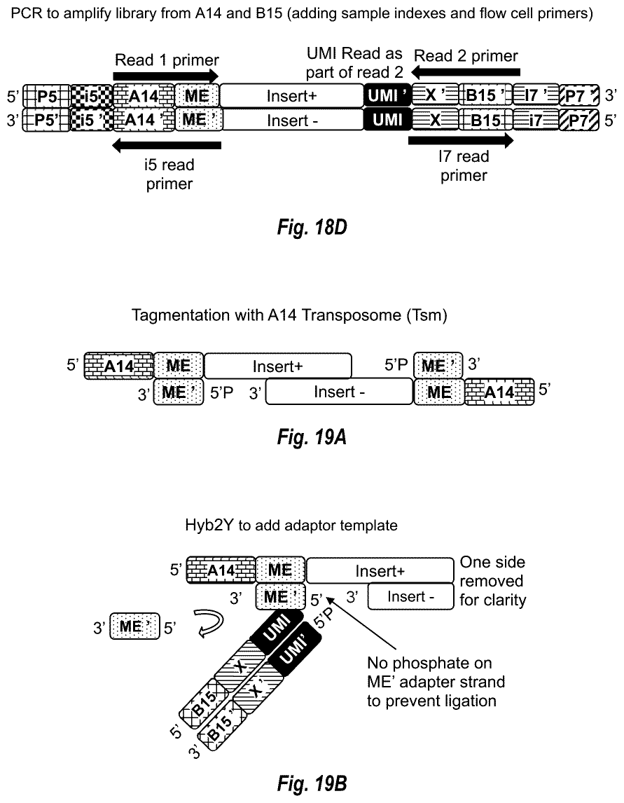

An exemplary adapter is shown in FIG. 18B. The adapter comprises a 5′ polymerase template switch oligonucleotide with the following from 5′ to 3′: B15, X, and UMI.

As described in Example 16c and shown in FIGS. 18A-C, an exemplary method of producing a UMI library with in-line UMIs comprises (1) applying a sample comprising double-stranded target nucleic acids to a transposome complex comprising: (i) a transposase, and (ii) a transposon comprising a first 3′ end transposon end sequence and a first adapter sequence; (2) tagmenting a first strand of the double-stranded target nucleic acids with the transposon to produce double-stranded target nucleic acid fragments comprising the first adapter sequence, (3) releasing the double stranded target nucleic acid fragments from transposome complex, (4) hybridizing a first polynucleotide comprising a UMI, and a second adapter sequence, (5) extending a second strand of the double-stranded target nucleic acid fragments, (6) copying the first polynucleotide, (7) producing double stranded target nucleic acid fragments comprising UMI, wherein the UMI is located between the double-stranded target nucleic acid fragments and the second adapter sequence, and (9) amplifying the double-stranded target nucleic acid fragments. The extending step described above involves a template switch from the target nucleic acid strand to the adapter strand.

The exemplary adapter and method described herein produces a UMI library wherein the UMI is adjacent to the 3′ end of the insert DNA (FIG. 18d). Using a standard sequencing method, each UMI and insert DNA sequence is captured using Read 2 without sequencing an ME sequence. The use of this exemplary adapter and method to produce a UMI library obviates the need for dark cycling when the UMI library is being sequenced.

H. Samples and Target Nucleic Acids

A biological sample used in accordance with the present disclosure can be any type that comprises target nucleic acids. However, the sample need not be completely purified, and can comprise, for example, nucleic acid mixed with protein, other nucleic acid species, other cellular components, and/or any other contaminant. In some embodiments, the biological sample comprises a mixture of nucleic acid, protein, other nucleic acid species, other cellular components, and/or any other contaminant present in approximately the same proportion as found in vivo. For example, in some embodiments, the components are found in the same proportion as found in an intact cell. In some embodiments, the biological sample has a 260/280 absorbance ratio of less than or equal to 2.0, 1.9, 1.8, 1.7, 1.6, 1.5, 1.4, 1.3, 1.2, 1.1, 1.0, 0.9, 0.8, 0.7, or 0.60. In some embodiments, the biological sample has a 260/280 absorbance ratio of at least 2.0, 1.9, 1.8, 1.7, 1.6, 1.5, 1.4, 1.3, 1.2, 1.1, 1.0, 0.9, 0.8, 0.7, or 0.60. Because the methods provided herein allow nucleic acid to be bound to solid supports, other contaminants can be removed merely by washing the solid support after surface bound tagmentation occurs. The biological sample can comprise, for example, a crude cell lysate or whole cells. For example, a crude cell lysate that is applied to a solid support in a method set forth herein, need not have been subjected to one or more of the separation steps that are traditionally used to isolate nucleic acids from other cellular components. Exemplary separation steps are set forth in Maniatis et al., Molecular Cloning: A Laboratory Manual, 2d Edition, 1989, and Short Protocols in Molecular Biology, ed. Ausubel, et al, hereby incorporated by reference.

In some embodiments, the sample that is applied to the solid support has a 260/280 absorbance ratio that is less than or equal to 1.7.

Thus, in some embodiments, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising nucleic acid.

In some embodiments, the sample is blood. In some embodiments, the sample is a cell lysate. In some embodiments, the cell lysate is a crude cell lysate. In some embodiments, the method further comprises lysing cells in the sample after applying the sample to a solid support to generate a cell lysate.

In some embodiments, the sample is a biopsy sample. In some embodiments, the biopsy sample is a liquid or solid sample. In some embodiments, a biopsy sample from a cancer patient is used to evaluate sequences of interest to determine if the subject has certain mutations or variants in predictive genes.

One advantage of the methods and compositions presented herein that a biological sample can be added to a flow cell and subsequent lysis and purification steps can all occur in the flow cell without further transfer or handling steps, simply by flowing the necessary reagents into the flow cell.

1. DNA

In some embodiments, the sample comprises a target double-stranded DNA. In some embodiments, the DNA is genomic DNA. In some embodiments, the DNA is cell-free DNA (cfDNA). In some embodiments, the DNA is circulating tumor DNA (ctDNA). In some embodiments, the DNA is a DNA:RNA duplex, which is discussed in detail in Section II.H.3 below.

2. RNA

In some embodiments, the sample comprises target RNA. In some embodiments, the sample comprises RNA and DNA. In some embodiments, the target RNA is mRNA. In some embodiments, the target RNA comprises coding, untranslated region (UTR), introns, and/or intergenic sequences

In some embodiments, the target RNA comprises a sequence complementary to at least a portion of one or more of the capture oligonucleotides.

In some embodiments, the target RNA is messenger RNA (mRNA), transfer RNA (tRNA), or ribosomal RNA (rRNA). Appropriate capture oligonucleotides could be designed based on the type of target RNA.

In some embodiments, the 3′ end of the target RNA binds to the capture oligonucleotides.

In some embodiments, the target RNA is mRNA. In some embodiments, the target RNA is polyadenylated (i.e., comprises a stretch of RNA that contains only adenine bases). In some embodiments, the mRNA comprises polyA tails. In some embodiments, the 3′ ends of the mRNA comprise polyA tails.

In some embodiments, the target mRNA comprises a polyA sequence and binds to capture oligonucleotides comprising polyT sequences.

3. DNA:RNA Duplex

In some embodiments, cDNA is synthesized from the sample comprising RNA as a first step of a library preparation. In other words, a DNA:RNA duplex may be generated in solution before tagmentation by a BLT. In some embodiments, the DNA:RNA duplex is then captured on a BLT by a capture oligonucleotide. In some embodiments, the DNA:RNA duplex bind directly to BLTs based on affinity for transposases comprised in transposome complexes.

In some embodiments, cDNA synthesis is performed by a reverse transcriptase. In some embodiments, this cDNA synthesis yield DNA:RNA duplexes, wherein a strand of DNA is generated that can hybridize to a strand of RNA. In some embodiments, a reverse transcriptase polymerase is added to a sample comprising RNA under conditions to synthesize cDNA. In some embodiments, conditions to synthesize cDNA include the presence of nucleotides and/or primers that can bind to RNA (such as polyT primers and/or randomer primers).

In some embodiments, the reverse transcriptase only prepares DNA from the RNA (without generating additional copies of the DNA to yield double-stranded DNA).

In some embodiments, DNA:RNA duplexes generated in solution can then be bound to BLTs and tagmented. As described in Section II.H.2 above on RNA, target RNA may comprise polyA tails that bind to capture oligonucleotides comprising polyT sequences.

In some embodiments, the fragments of the DNA:RNA duplexes can be used to generate sequences of coding, untranslated region (UTR), introns, and/or intergenic sequences of the target RNA.

In some embodiments, a method of preparing an immobilized library of tagged DNA:RNA fragments from target RNA comprises adding a reverse transcriptase polymerase to a sample comprising target RNA under conditions to synthesize cDNA and generate DNA:RNA duplexes; immobilizing DNA:RNA duplexes to a solid support having transposome complexes immobilized thereon, wherein the transposome complexes comprise a transposase bound to a first polynucleotide comprising a 3′ portion comprising a transposon end sequence, and a first tag; wherein the sample is applied to the solid support under conditions wherein the DNA:RNA duplexes bind to capture oligonucleotides or transposases directly; and fragmenting the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

### III. Methods of Sequencing UMI Libraries

The present disclosure further relates to sequencing of the UMI libraries produced according to the methods provided herein. The UMI libraries can be sequenced according to any suitable sequencing methodology, such as direct sequencing, including sequencing by synthesis, sequencing by ligation, sequencing by hybridization, nanopore sequencing and the like. In some embodiments, the library is sequenced on a solid support. In some embodiments, the solid support for sequencing is the same solid support upon which the surface bound tagmentation occurs. In some embodiments, the solid support for sequencing is the same solid support upon which the amplification occurs.

One exemplary sequencing methodology is sequencing-by-synthesis (SBS). In SBS, extension of a nucleic acid primer along a nucleic acid template (e.g., a target nucleic acid or amplicon thereof) is monitored to determine the sequence of nucleotides in the template. The underlying chemical process can be polymerization (e.g., as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template.

Flow cells provide a convenient solid support for housing amplified DNA fragments produced by the methods of the present disclosure. One or more amplified DNA fragments in such a format can be subjected to an SBS or other detection technique that involves repeated delivery of reagents in cycles. For example, to initiate a first SBS cycle, one or more labeled nucleotides, DNA polymerase, etc., can be flowed into/through a flow cell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, e.g., in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be adapted for application of pyrosequencing to amplicons produced according to the present disclosure are described, e.g., in WIPO Patent App. Ser. No. PCT/US11/57111, US 2005/0191698 A1, U.S. Pat. Nos. 7,595,883, and 7,244,559, each of which is incorporated herein by reference.

Some embodiments can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, e.g., in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, e.g., Deamer et al. Trends Biotechnol. 18, 147-151 (2000); Deamer et al. Acc. Chem. Res. 35:817-825 (2002); Li et al. Nat. Mater. 2:611-615 (2003), the disclosures of which are incorporated herein by reference). In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008), the disclosures of which are incorporated herein by reference).

Exemplary methods for array-based expression and genotyping analysis that can be applied to detection according to the present disclosure are described in U.S. Pat. Nos. 7,582,420; 6,890,741; 6,913,884 or U.S. Pat. No. 6,355,431 or US Patent Pub. Nos. 2005/0053980 A1; 2009/0186349 A1 or US 2005/0181440 A1, each of which is incorporated herein by reference.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more nucleic acid fragments, the system comprising components such as pumps, valves, reservoirs, fluidic lines and the like. A flow cell can be configured and/or used in an integrated system for detection of target nucleic acids. Exemplary flow cells are described, e.g., in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

In some embodiments, a method of sequencing a UMI library of the present disclosure comprises sequencing the UMIs to provide increased sensitivity in DNA sequencing. In some embodiments, the sequencing method comprises NextSeq 500/550 (Illumina).

A. Dark Cycles

In some embodiments, a custom sequencing recipe was prepared and selected using the NextSeq software to comprise dark cycles, which are used to skip the recording of a particular sequence. The sequencing chemistry of that sequence is still carried out, but the sequencing is not imaged by the instrument. Dark cycles are used to mitigate phasing/prephasing issues relating to repeatedly sequencing low diversity sequences, such as a library of ME sequences, that may globally worsen the sequencing result. After the dark cycles, the imaging of sequences is resumed so that the insert sequences of the target nucleic acids are recorded.

A custom sequencing recipe comprised modifying a standard recipe to include an appropriate number of dark cycles to span the length of the sequence to be skipped over. In other words, the number of dark cycles is equal to the number of bases intended to be skipped over. For example, if the sequence to be skipped over is an ME sequence, which is 19 bases long, 19 dark cycles are used. In some embodiments, the sequence to be skipped over is an ME sequence. In embodiments with a 19-nucleotide long ME, the number of dark cycles is 19. With a ME having a different number of nucleotides, the dark cycle is generally the number of nucleotides. To get the maximum benefit from a dark cycle, a user can skip the entire ME; however, it is also possible to skip the majority of the ME domain and sequence part of it, ignoring those nucleotides in the result.

In some embodiments, the sequencing method comprises dark cycles wherein data is not being recorded for a portion of the sequencing method. In some embodiments, the data not being recorded is sequence data associated with the 3′ transposon end sequence. In some embodiments, the sequence data not being recorded is an ME sequence. In some embodiments, the dark cycles comprise 19 cycles.

In some embodiments, the sequencing method does not comprise dark cycles. In these embodiments, the method of preparing a UMI library obviates the need for dark cycles because each UMI is adjacent to the 3′ end of the insert nucleic acids without an ME sequence between them (FIG. 20).

In some embodiments, custom primers are used to obviate the need for dark cycles. In these embodiments, the custom primers are bridged primers that comprise a sequence that aligns with ME (FIGS. 4 and 6B). In these embodiments, the ME sequence is not imaged.

B. Sequencing Primers

Sequencing primers and adapter sequences that may be used for sequencing UMI libraries with Illumina library preparation kits and sequencing platforms, e.g., Nextera, Illumina Prep, Ilumina PCR, AmpliSeq™, TruSight®, and TruSeq™, are as disclosed in Illumina Adapter Sequences Document #1000000002694 v15, and is hereby incorporated by reference in its entirety. These sequencing primers and adapters may be modified in accordance with the present disclosure. Examples of said primers and adapters include the following: Read 1, Read 2, Index 1 Read, Index 2 Read, Index 1 (i7) Adapters, Index 2 (i5) Adapters, Index Adapters 1-27, TruSeq Universal Adapter, Index PCR Primers, Multiplexing Adapters, Multiplexing Read Sequencing Primers, Multiplexing Index Read Sequencing Primers, and PCR Primer Index Sequences 1-12.

In some embodiments, the sequencing method comprises binding sequencing primers having similar melting temperatures.

1. Custom Primers

Custom primers may be used in sequencing reactions to serve different functions.

In some embodiments, UMI sequences are included in custom primers to allow for primer binding to UMIs.

In some embodiments, a custom primer may comprise sequences which serve to lengthen the primer and/or affect the melting temperature of the primer. In some embodiments, the custom sequencing primers and the standard sequencing primers that may be used in the same reaction may have similar melting temperatures.

In some embodiments, the custom primer is a bridged primer comprising one or more spacers. A spacer allows the bridged primer to align with any nucleic acid sequence.

In some embodiments, the spacer may bind to a target nucleic acid sequence. In some embodiments, the spacer comprises a universal hybridization sequences, such as inosines.

In some embodiments, the spacer may align with a target nucleic acid sequence without binding to it. In some embodiments, the spacer comprises a non-nucleic acid linker.

In some embodiments, the spacer aligns with a variable sequence. In some embodiments, the space aligns with a UMI sequence. In some embodiments, the spacer aligns with a UDI sequence.

In some embodiments, the sequencing primer comprises sequence completely or partially complementary to one or more unique primer binding sequences. In some embodiments, the sequencing primer comprises at least an A2 sequence, at least an A14 sequence, or at least a B15 sequence.

In some embodiments, the unique primer binding sequence is A2, A14, and/or B15.

a) Spacers

As used herein, a spacer region in a sequence refers to a nucleic acid sequence not carrying any structural or codifying information for known gene functions. The spacer region on a polynucleotide or an oligonucleotide is capable of aligning with varied sequences. In some embodiments, a spacer region is capable of aligning with a range of i5 sequences, which are disclosed in Illumina Adapter Sequences Document #1000000002694 v15 and are incorporated herein by reference. In some embodiments, the spacer region aligns with a UMI sequence. In some embodiments, the spacer region aligns with an ME sequence.

In some embodiments, the spacer region is a universal sequence. In some embodiments, the spacer region is a non-DNA spacer. In some embodiments, the spacer region includes universal bases, such as inosines or nitroindoles. Alternatively, the spacers may comprise a synthetic linker. Examples of synthetic linkers include C3 Spacer, hexanediol, 1′,2′-dideoxyribose (dSpacer), Photo-Cleavable Spacer (PC Spacer), Spacer 9, and Spacer 18. C3 Spacer is a C3 Spacer phosphoramidite that can be incorporated internally or at the 5′-end of the oligonucleotide. Multiple C3 Spacers can be added at either end of an oligonucleotide to introduce a long hydrophilic spacer arm for the attachment of fluorophores or other pendent groups. Hexanediol is a 6-carbon glycol spacer that is capable of blocking extension by DNA polymerases. This 3′ modification is capable of supporting synthesis of longer oligonucleotides. The dSpacer modification can be used to introduce a stable abasic site within an oligonucleotide. PC Spacer can be placed between DNA bases or between the oligonucleotide and a 5′-modified group. PC Spacer offers a 10-atom spacer arm which can be cleaved with exposure to UV light in the 300 to 350 nm spectral range. Cleavage releases the oligonucleotide with a 5′-phosphate group. Spacer 9 is a triethylene glycol spacer that can be incorporated at the 5′-end or 3′-end of an oligonucleotide or internally. Multiple insertions can be used to create long spacer arms. Spacer 18 (iSp18) is an 18-atom hexa-ethyleneglycol spacer and can be considered as the longest spacer arm that can be added as a single modification.

In some embodiments, the spacer includes an iSp18 linker. An iSp18 linker, as used herein, is a standard modification linker having C18 spacers (an 18-atom hexa-ethylene glycol spacer), and is equivalent to 4 base pairs in length. Thus, a 2×sp18 linker is equivalent to 8 base pairs in length. In some embodiments, the spacer region comprises a 2×iSp18 synthetic linker. In some embodiments, the spacer region comprises one or more C18 spacers, such as 1, 2, 3, 4, 5, 6, or more C18 spacers. In some embodiments, the spacer region comprises two C18 spacers (which are equivalent in length to 8 nucleotides). In some embodiments, the spacer is a C9 spacer equivalent in length to 2 base pairs. In some embodiments, the spacer region comprises one or more C9 spacers (triethyleneglycol spacer), such as 1, 2, 3, 4, 5, 6, or more C9 spacers. In some embodiments, the spacer is a conventional spacer used with existing indices, such as a 10-base pair spacer. In some embodiments, the spacer region is a combination of spacers, for example, a combination of one or more C18 spacers and one or more C9 spacers, or any combination of any spacer described herein. In some embodiments, the spacer region is a length equivalent to 2, 3, 4, 5, 6, 7, 8, 9, 10, 12, 15, 20, or 30 base pairs. In some embodiments, the spacer region is a length approximately equivalent to 8 or 10 base pairs or nucleotides. In some embodiments, the spacer region is specifically chosen to be the same length as the index region. In some embodiments, the index regions are 8 nucleotides long, and the spacer region comprises two C18 spacers. In some embodiments, the index regions are 10 nucleotides long and the spacer region comprises two C18 spacers and one C9 spacer.

In some embodiments, the spacer includes abasic nucleotides. An abasic nucleotide can be introduced at any position in the spacer. Examples of spacers with abasic nucleotides include dSpacer (1′,2′-dideoxyribose; DNA abasic), rSpacer (i.e., RNA abasic), and Abasic II. In some embodiments, the dSpacer is an abasic furan, tetrahydrofuran (THF), THF derivative, or apurinic/apyrimidinic (AP) nucleotide.

In some embodiments, the spacer includes wobble bases. A wobble base can be introduced at any position in the spacer. A wobble base pair is a pairing between two nucleotides that do not follow Watson-Crick base pair rules, such as guanine-uracil, hypoxanthine-uracil, hypoxanthine-adenine, and hypoxanthine-cytosine.

### IV. Kits Comprising a Transposome Complex

In some embodiments, a kit comprises components of transposome complexes disclosed herein. In some embodiments, the kit comprises the components for generating said transposome complexes, including transposases and oligonucleotides comprising transposons, 5′ and 3′ transposon end sequences, adapter sequences, UMI sequences, and/or other HYB/HYB′ sequences.

A kit may comprise any of a variety of adapters. In many embodiments, adapters may be chosen from 3′ adapters, polynucleotide adapters, forked adapters, hairpin UMI adapters, hairpin UMI and universal hybridizing tail adapters, splint ligation adapters, template switch oligonucleotide adapters, and any suitable oligonucleotide.

In some embodiments, a kit may comprise components for Hyb2Y, such as adapters and buffers

In some embodiments, a kit may comprise solid support such as beads.

In some embodiments, a kit may comprise a reverse transcriptase polymerase.

In some embodiments, a kit may comprise sequencing primers.

### EXAMPLES

The examples that follow describe methods that relate to preparing DNA sequencing libraries with UMIs. The generation of sequencing libraries using the BLT method (such as Illumina DNA Prep (Research Use Only, RUO), previously known as Nextera DNA Flex Library Prep, and Nextera XT DNA Library Preparation Kits) is a convenient and efficient approach that is compatible with NGS library preparation workflows. For many of these, it is desirable to track relative orientation and uniqueness of sequenced DNA molecules (i.e., the strandedness or directionality of the target DNA) and to be able to resolve them bioinformatically. The methods described in the examples relate to the use of UMIs to provide strandedness or directionality, which is a feature not afforded by the current generation of BLT methods. The UMIs are incorporated without using Illumina TruSeq™ methods. The following examples disclose different ways of incorporating the UMIs.

### Example 1. Preparation of a DNA Library for Sequencing Using a UMI-BLT to Enable Duplex UMI Error Correction

This example describes an asymmetrical tagmentation BLT method used to prepare a DNA sequencing library with unique dual indexes (UDIs) and duplex UMIs. This example describes a method that combines UDIs and UMIs for error correction. A single UMI is used to tagment the DNA library, and the single UMI is subsequently copied to produce a duplex UMI.

The method of this example combined the BLT method with the Hyb2Y workflow. In the tagmentation step, a first UMI was added to the first strand of target DNA and a second UMI was added to the second strand of target DNA.

In this method, an additional A2 adapter sequence was added to the transposon arm in the BLT and the Hyb2Y workflow was used to copy the UMI. The addition of the A2 sequence to the BLT adapter serves two purposes. First, it allows the annealing of a Hyb2Y oligonucleotide that can be extended to have a paired UMI on the opposite strand. Hybridization of the Hyb2Y oligonucleotide to A2 allows for a longer extension that can copy the UMI and adapter sequences rather than relying on other methods where the extension is minimal. Second, the A2 sequence enables the development of custom sequencing recipes and custom primers for sequencing that have the same annealing temperature (Tm) as the standard sequencing primers. Further, a library prepared according to this method reduces the amount of adapter dimer that is sometimes observed when forked adapter BLT designs are used. By circumventing adapter dimers, this method also increases library yield.

A. Materials

The following materials were used in this example: (1) genomic DNA (gDNA) Horizon Tru-Q 7 Reference Standard (Horizon Catalog #HD734); (2) Illumina DNA Prep with Enrichment (IDPE; Illumina Catalog #20025523 and 20025524; previously Nextera Flex for Enrichment); (3) TruSight Oncology UMI Reagents (Illumina Catalog #20024586); (4) TruSight Tumor 170 reagents (Illumina Catalog #20028821); (5) New Enrichment Blocker NHB2 (Illumina Reference #20031771); (6) Extension Ligation Mix ELM3 (Illumina Catalog #20019117); (7) NextSeq 500/550 v2.5 Kit (Illumina Catalog #20024906); and (8) custom primers.

B. BLT Library with Duplex UMIs

In this method, BLTs for tagmenting target DNA fragments were first prepared in a reaction mixture with capture oligonucleotides that comprise a UMI-BLT (FIG. 1). Target DNA for tagmentation was added to a reaction mixture with UMI-BLTs (FIG. 2). 10 ng and 50 ng of gDNA Horizon Tru-Q 7 Reference Standard were used as target DNA.

A tagmented library containing AB-Long single UMIs was prepared with BLTs that were made at similar density to eBLTs used in IDPE. The library was prepared according to IDPE protocol guidelines, using TruSight™ Tumor (TST170; Illumina) probes. Stop tagmentation buffer ST2 was added to stop the tagmentation process.

The resulting tagmented library was heated for 5 minutes at 55° C. to release the tagmented library into solution. The 3′-biotinylated ME remained bound to the beads and was not transferred. The reaction mixture was incubated at room temperature for 5 minutes and the reaction mixture was washed twice with tagment wash buffer (TWB).

Then, the Hyb2Y oligonucleotide (5′P-A2′A14′-3′ in FIG. 2) was added and annealed at 65° C. for 10 minutes. The reaction mixture was allowed to slowly cool to 37° C. Then, the supernatant of the reaction mixture was removed and mixed with the extension-ligation mix ELM3 for gap-filling.

Thirty-four bases are gap-filled by extension and ligation in ELM3 for 30 minutes at 37° C. The UMI sequence was copied during this step, which enables UMI duplex error correction by allowing one to identify and group the top strands and the bottom strands using the UMI. Then, solid phase reversible immobilization beads (SPRI) were used to clean up the reaction mixture to produce a solution with tagmented DNA. Nine cycles of PCR were performed using UDI primers to amplify the tagmented DNA. The PCR products were then purified using SPRI to capture tagmented DNA that fall within the correct size range. Finally, the library (about 500 ng of DNA) was enriched using IDPE and TST170 probes. An additional blocker was added for the hybridization of AB-Long BLT probes.

These steps produced a standard structure BLT library with duplex UMIs. The library comprised A14 and B15 oligonucleotide sequences that may be used for PCR amplification with Illumina UDIs (FIG. 2).

C. BLT Library with Single UMIs

A second BLT library was prepared. This library comprised single UMIs and were produced using A-B-short single UMIs. The library was prepared using the steps described above for A-B-long single UMIs except that no additional blocker was used for BLT hybridization.

D. Control Libraries

For comparison, a separate tagmented library was prepared using TruSight Oncology UMI Reagents according to TruSight Tumor 170 protocol guidelines.

For further comparison, a library without UMIs was prepared using NFE.

### Example 2. Sequencing a DNA Library Comprising Duplex UMI with Dark Cycles

This example describes a method of sequencing the DNA libraries of Example 1.

A. Materials

The following systems and materials were used in this example: (1) NextSeq 500 sequencing system were used (Illumina Document #15046563); and (2) sequencing primers and custom primers, where needed, specific to libraries of Example 1 (Illumina Document #15057456).

B. Methods

The libraries from Example 1 were pooled, denatured, and added to NextSeq 500 sequencing cartridges according to protocol guidelines. Custom primers were diluted and added to the relevant positions in the cartridge following NextSeq 500 and NextSeq 550 Sequencing Systems Custom Primers Guide.

A custom sequencing recipe was loaded to the sequencing instrument and selected using the NextSeq software. The recipe comprised modifying a standard recipe to include 19 dark cycles over the ME region. Dark cycles are sequencing cycles with no imaging, which corrected for phasing/prephasing issues that may globally worsen the sequencing result. Dark cycles are discussed in detail in Section III.A above. During the dark cycles, the 19 bases of the ME region were not imaged. After the dark cycles, imaging resumed and the insert sequences were imaged.

The sample sheet included settings as found in the TruSight Oncology UMI Reagents guide.

Data analysis was performed on Basespace Sequence Hub using internal UMI collapsing APP and Dragen Enrichment App.

1. Primers

The custom sequencing primers used are as shown in FIG. 3B. The 4 custom primers comprised melting temperatures (Tm) that are compatible with standard sequencing primers and can therefore be mixed and used in the same sequencing reactions. The custom primers, as shown FIG. 3B, were as follows: (1) Custom Primer 1 UMI+Read 1, (2) Custom Primer i5, (3) Custom Primer i7, and (4) Custom Primer 4 UMI+Read 2. The custom primers were designed to anneal to their respective regions as indicated by the blue arrows in FIG. 3B. Custom Primer 1 UMI+Read 1 annealed to the A14-A2 sequence. Custom Primer i5 annealed to the A14′-A2′ sequence. Custom Primer i7 annealed to the A2′-B15′ sequence. Custom Primer 4 UMI+Read 2 annealed to the B15-A2 sequence. The sequence of the insert DNA was read with Custom Primer 1 UMI+Read 1 and Custom Primer 4 UMI+Read 2.

Three custom primer ports containing a total of six primers were used for this sequencing method. The i7 and i5 custom primers were added to one custom primer port as per standard operating procedures for sequencing. The primers used and prepared according to this example may be useful for one skilled in the art who may have a limited number of available primer ports on a sequencing cartridge. For example, some sequencing platforms have only three primer ports available. This method allows for the mixing of different custom sequencing primers in a single reaction to be used at different times during the sequencing process, thereby allowing one skilled in the art to minimize the number of custom primer ports needed on a sequencing cartridge.

Optionally, the method may instead, comprise only two primers—Custom Primer 1 UMI+Read 1 and Custom Primer 2 UMI+Read 2. These two primers can be pre-mixed and require only two custom primer ports.

C. Results

FIG. 3C shows the quality score for every cycle in the sequencing run. Briefly, a quality score is a prediction of the probability of an error in base calling. A high-quality score implies that a base call is more reliable and less likely to be incorrect. For base calls with a quality score of Q30, one base call in 1,000 is predicted to be incorrect. When sequencing quality reaches Q30, virtually all of the reads will be perfect having zero errors and ambiguities. Q30 is considered a benchmark for quality in next-generation sequencing.

While FIG. 3C shows %≥Q30, FIG. 3D shows the intensity of sequencing cycle for every cycle in the sequencing run of this example. Dark cycles were used to speed up sequencing and avoid recording uninformative images of the reactions that span the adapter sequences. The dark cycles (and light cycles) reduce the quality of the subsequent sequencing (FIGS. 3C and 3D) compared to starting a new read at the insert.

In sequencing reactions with 50 ng of template input, the TruSight UMI method demonstrated superior performance. It is possible that they Hyb2Y workflow in Example 1 needed optimization to enable improved sequencing performance.

As shown in FIG. 3E, the TruSight UMI method (TruSight-Duplex) demonstrated superior performance in reactions with 50 ng of template input. This may have been caused by UMI reads being discarded at the first step of the analysis due to errors introduced into the UMI sequence by the polymerase used during the extension and ligation step in Example 1. In FIG. 3E, designs that do not have duplex UMIs were called as zero. Adapter blocking for the fork-duplex libraries were also suboptimal. Regardless, the Fork-Duplex dataset had called 20% duplex families. This number should improve with optimizations to the biochemistry in the Hyb2Y workflow of Example 1. Examples of parameters that may be optimized include oligonucleotide concentrations, time for hybridization, temperature for hybridization, and choice of sequence used for hybridization.

### Example 3. Sequencing a DNA Library Comprising Duplex UMI with Bridged Primer Rehybridization

This example describes a method of sequencing the DNA libraries of Example 1.

A. Materials

The materials are as described in Example 2 above.

B. Methods

The methods are as described in Example 2 above with the following modifications.

A custom sequencing recipe is used here that does not comprise dark cycles. The recipe further comprises an additional primer rehybridization during read 1 and read 4 (FIG. 4).

1. Primers

Custom primers in this example are as provided in Table 2 and FIG. 4. The primers for Read 1 and Read 6 are bridged primers.

TABLE 2
Custom sequencing primers

| | | Primer | Primer | SEQ ID |
|---|---|---|---|---|
| Read | Primer Sequence | name | purpose | NO |
| 1 | TCGTCGGCAGCGTCTCACTCAAGAAC | A14-A2 | Custom UMI | 8 |
| | AGC | | 1 Read | |
| 2 | TCGTCGGCAGCGTCTCACTCAAGAAC | A14-A2- | Custom | 9 |
| | AGC/iSp18// | spacer- | Bridged | |
| | iSp18/AGATGTGTATAAGAGACAG | ME | Primer for | |
| | | | Insert 1 Read | |
| 3 | GCTGTTCTTGAGTGACCGAGCCCACG | A2′-B15′ | Custom i7 | 10 |
| | AGAC | | Read | |
| 4 | GCTGTTCTTGAGTGAGACGCTGCCGA | A2′-A14′ | Custom i5 | 11 |
| | CGA | | Read | |
| 5 | GTCTCGTGGGCTCGGTCACTCAAGAA | B15-A2 | Custom UMI | 12 |
| | CAGC | | 2 Read | |
| 6 | GTCTCGTGGGCTCGGTCACTCAAGAA | B15-A2- | Custom | 13 |
| | CAGC/iSp18// | spacer- | Bridged | |
| | iSp18/AGATGTGTATAAGAGACAG | ME | Primer for | |
| | | | Insert 2 Read | |

iSp18 = an 18-atom hexa-ethyleneglycol spacer between two oligonucleotides; may be used for Illumina sequencing.

Each bridged primer comprises a sequence that anneals to the A14-A2 sequence, two spacers that span but do not anneal to the UMI sequence, and a sequence that anneals t the ME sequence. In the tagmented library, the A14-A2 and ME sequences are constant sequences while the UMI sequence varies. In this example, two copies of iSp18 are used are the two spacers in each of primers 2 and 6.

In the sequencing method of this example, primer 1 first anneals and is then removed for primer 2 to anneal. Similarly, primer 5 anneals before it is removed for primer 6 to anneal. The sequence of the insert DNA was read with Custom Bridged Primer for Insert 1 Read and Custom Bridged Primer for Insert 2 Read.

### Example 4. Preparation of a DNA Library for Sequencing Using a UMI-BLT to Enable Duplex UMI Error Correction

This example describes an asymmetrical tagmentation BLT method used to prepare a DNA sequencing library with UDIs and duplex UMIs for error correction. The materials are as described in Example 1. In the tagmentation step, a UMI was added to the first strand of target DNA; the second strand of target DNA was not tagmented with a UMI.

In this method, the transposome structure comprising UMI-BLT for tagmenting target DNA are as shown in FIG. 5A. Tagmented DNA is processed as shown in FIG. 5B. The tagmented DNA is washed with sodium dodecyl sulfate (SDS) and the transposases, TsTn5, (shown in FIGS. 5A and 5B) are removed. The tagmented DNA library is amplified by PCR using UDI primers.

### Example 5. Sequencing a DNA Library Comprising Duplex UMI with Dark Cycles

This example describes a method of sequencing the DNA library of Example 4 which comprised dark cycles (FIG. 6A).

A. Materials

The materials are as described in Example 2 above.

B. Methods

The methods are as described in Example 2 above with the following modifications.

1. Primers

In this method, 4 primers were used: (1) Standard Insert Read 1, (2) Custom i7, (3) Standard i5, and (4) UMI+Insert Read 2. The primers were designed to anneal to their respective regions as indicated by black arrows in FIG. 6A. Standard Insert Read 1 annealed to the A14-ME sequence. Custom i7 annealed to the A2′-B15′ sequence. Standard i5 annealed to the ME′-A14′ sequence. UMI+Insert Read 2 annealed to the B15-A2 sequence.

C. Results

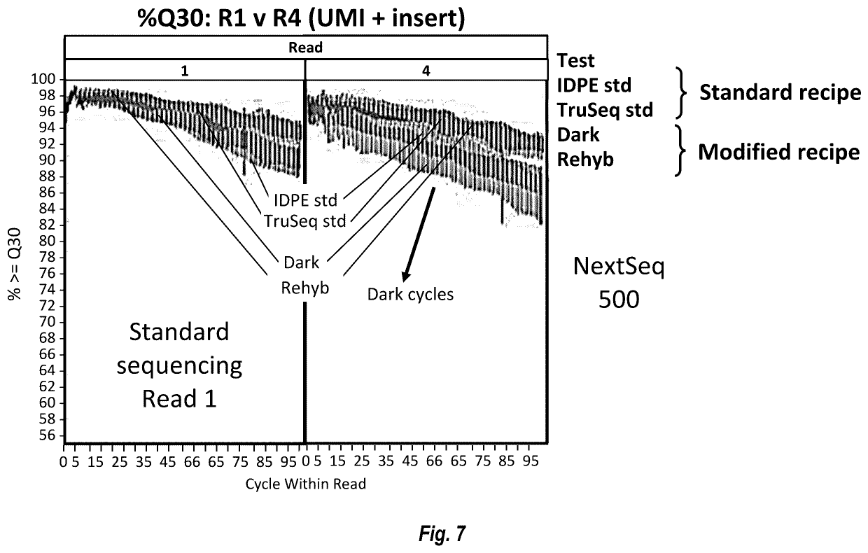

The sequencing method of this example (FIG. 6A) was compared to sequencing runs using the TruSeq™ method or IDPE standard method (FIG. 3A). % Q30 for the standard sequencing Read 1 and R4 UMI+Insert Read 2 for the current method as shown in FIG. 7 (“Dark”) indicate that although the method did not perform as well as the IDPE (“IDPE std”) and TruSeq™ (“TruSeq std”) methods, the current method was successful. A decrease in % Q30 scores was also observed after dark cycles. This sequencing method uses only three primers and may be a preferred method when used with sequencing instruments with cartridges that can support no more than three primers.

### Example 6. Sequencing a DNA Library Comprising Duplex UMI with Bridged Primer Rehybridization

This example describes a method of sequencing the DNA library of Example 4 which comprises bridged primer rehybridization instead of dark cycles (FIG. 6B).

A. Materials

The materials are as described in Example 5 above.

B. Methods

The methods are as described in Example 5 above with the following modifications.

1. Primers

In this method, 5 primers are used: (1) Standard Insert Read 1, (2) Custom i7, (3) Standard i5, (4) UMI, and (5) Insert Read 2 Bridged Primer. The primers were designed to anneal to their respective regions as indicated by black arrows in FIG. 6B. Primers (1) to (4) anneal to the regions described in the preceding paragraph. Primer 5 comprises a sequence that anneals to the A2-B13 sequence, a spacer that spans but does not anneal to the UMI sequence, and a sequence that anneals to the ME sequence. Primer 5 obviates the need for dark cycling in the sequencing method. In this method, primer 4 first anneals and is then removed for primer 5 to anneal. The sequence of the insert DNA is read with Standard Insert Read 1 and Insert Read 2 Bridged Primer.

C. Results

The sequencing method of this example (FIG. 6B) was compared to sequencing runs using the TruSeq™ method or IDPE standard method (FIG. 3A). % Q30 for the standard sequencing Read 1 and R5 Insert Read 2 Bridged Primer for the current method as shown in FIG. 7 (“Rehyb”) indicate that the method performed as well as the TruSeq™ (“TruSeq std”) and IDPE (“IDPE std”) methods and provided better sequencing quality than the method with dark cycles (“Dark;” also see Example 5).

### Example 7. Preparation of a DNA Library from Cell-Free DNA (cfDNA) with UMI BLT for Sequencing

This example describes an asymmetrical tagmentation BLT method used to prepare a DNA sequencing library with UDIs and duplex UMIs for error correction. The materials are as described in Example 1. In the tagmentation step, a first UMI was added to the first strand of target DNA and a second UMI was added to the second strand of target DNA.

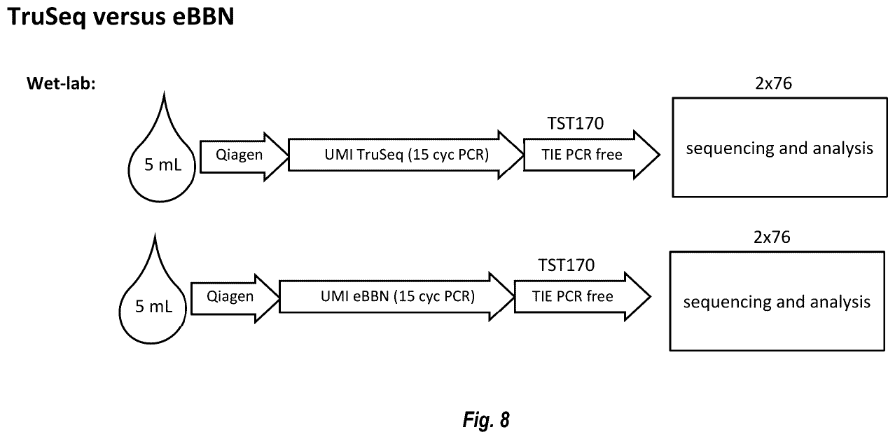

cfDNA was extracted from 5 mL of plasma from a single patient. cfDNA was extracted using Mg²⁺-free BLT Tn5. As shown in FIG. 8, cfDNA was processed using the TruSeq™ workflow as a control or was processed using the method described in this example (“eBBN” in FIG. 8).

First, the cfDNA was processed using TruSeq™ workflow as follows: (1) end repair for 30 minutes, (2) A-tailing for 30 minutes, (3) ligation of UMIs for 30 minutes, (4) ligation of adapters for 30 minutes, (5) SPRI cleanup, and (6) amplification by PCR.

A separate sample of cfDNA was processed according to the tagmentation workflow for the current method, as shown in FIG. 9, with the following steps: (1) cfDNA was tagmented with capture oligonucleotides comprising single UMI adapters for 5 minutes, (2) tagmentation was stopped, (3) the tagmented cfDNA, i.e., the UMI library, was washed using 5- to 10-minute washes, and (4) the UMI library that was produced was amplified by PCR.

In this method, the UMIs were added to the BLT capture oligonucleotides in place of the UDIs, which precludes additional indexing using UDIs. The UMIs are not on the same strand as the strand with the BLT capture moiety; the UMIs are on the transferred strand while the BLT capture moiety is on the non-transferred strand.

Ten UMI sequences were used to the i7 position and 10 UMI sequences were used in the i5 position. Tagmented DNA fragments were gap-filled and amplified by PCR using P5 and P7 primers. This method produced a standard structure BLT library with A14 and B15 oligonucleotide sequences ready for sequencing using standard sequencing primers Example 8. Sequencing a DNA Library Comprising Single UMIs

This example describes a method of sequencing the DNA library of Example 7.

A. Materials

The materials are as described in Example 2 above.

B. Methods

The methods are as described in Example 2 above with the following modifications.

1. Primers

This example comprised a standard sequencing run and standard sequencing primers Nextera Read primer 1 (NR1 read), i7 read, i5 read, and Nextera Read primer 2 (NR2 read). The primers were designed to anneal to their respective regions as indicated by black arrows in FIG. 9. Because the i7 and i5 regions have been usurped by UMIs, the UMIs were captured from the index read.

C. Results

Even distribution of UMI reads across the DNA library indicate that single UMIs were successfully incorporated in the tagmented DNA fragments (FIG. 10). A Read Collapsing analysis step was performed on the sequencing reads to group duplicate reads and collapse them into a single consensus aligned read. The resulting reads, deduped reads, have higher per-base quality and lower noise from various sources. Read Collapsing is a useful metric for quality control when UMIs are involved.

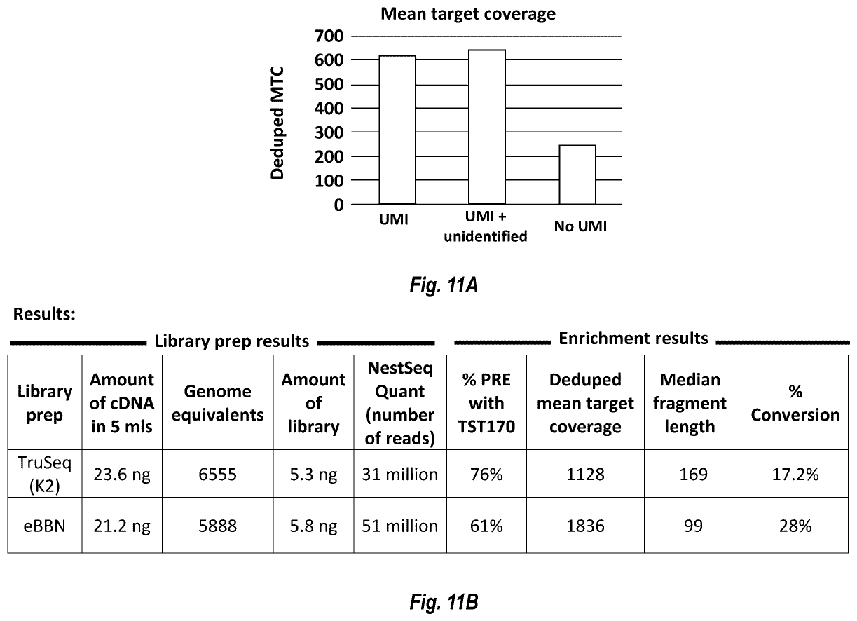

As shown in FIGS. 11A and 11B, a single UMI-BLT library (shown as “eBBN” in FIG. 11B) has greater deduped mean target coverage and higher conversion of cfDNA to library than a TruSeq™ library (shown as “No UMI” in FIG. 11A).

### Example 9. Preparation of a DNA Library Using Duplex UMI-BLT for Sequencing with UDIs and Duplex Sequence Error Correction

This example describes a symmetrical tagmentation BLT method used to prepare a DNA sequencing library with UDIs and duplex UMIs for error correction. The materials are as described in Example 1. The method comprises duplex UMIs in forked adapter capture oligonucleotides for BLT (FIG. 12). In the tagmentation step, UMIs are added to both strands of target DNA.

First, a pool of UMIs comprising 120 different UMI duplexes is formed. Each UMI duplex is prepared separately and then mixed together to form the pool of UMIs. The pool is used to prepare forked adapter capture oligonucleotides, which are then used to prepare a universal UMI BLT (universal UMI Tsm). Target DNA fragments are tagmented using the universal UMI Tsm. Gap-filling and ligation are carried out with ELM. The tagmented DNA are amplified by PCR using Nextera Index primers and are ready for sequencing.

### Example 10. Sequencing a DNA Library Comprising Duplex UMIs and UDIs

This example describes a method of sequencing the DNA library of Example 9 which comprises duplex UMIs and UDIs. This method includes the use of four standard primers and dark cycles to avoid imaging the ME regions.

A. Materials

The materials are as described in Example 2 above.

B. Methods

The methods are as described in Example 2 above with the following modifications.

1. Primers

This example comprises a sequencing run with 19 dark cycles and sequencing primers (1) A14 Read, (2) i7 Read, (3) B15 Read, and (4) i5 Read. The primers were designed to anneal to their respective regions as indicated by grey arrows in FIG. 12.

The standard A14 read and B15 read primers anneal to A14 and B15 regions. These regions comprise short nucleotide sequences (i.e., 14 base pairs), which results in the design of low Tm for the A14 read and B15 read primers. The primers benefit from modifications, such as an additional 10 base pairs, that increase their respective Tins so that they UMI sequences may be read.

### Example 11. Preparation of a DNA Library for Sequencing Enabling Indexing and Duplex Sequence Error Correction

This example describes a symmetrical tagmentation BLT method used to prepare a DNA sequencing library with UDIs and duplex UMIs for error correction. The materials are as described in Example 1. The method comprises UMIs in forked adapter capture oligonucleotides for BLT (FIG. 13). In the tagmentation step, UMIs are added to both strands of target DNA.

Steps for preparing UMIs, BLTs, and tagmented DNA are as described above in Example 9.

### Example 12. Sequencing a DNA Library

This example describes a method of sequencing the DNA library of Example 11.

A. Materials

The materials are as described in Example 2 above.

B. Methods

The methods are as described in Example 2 above with the following modifications.

1. Primers

This example comprises 6 custom sequencing primers: (1) Custom 1, (2) Custom UMIi7, (3) Custom i7, (4) Custom 2, (5) Custom UMIi5, and (6) Custom i5. The primers were designed to anneal to their respective regions as indicated by black arrows in FIG. 13.

### Example 13. Preparation of a DNA Library for Sequencing Using a 3′ Adapter Comprising a Hairpin UMI and a Universal Hybridizing Tail

This example describes an asymmetrical tagmentation BLT method used to prepare a DNA sequencing library with UMIs wherein the UMI is incorporated after tagmentation (FIG. 14). A 3′ adapter comprising a hairpin-UMI and universal hybridizing tail is used to incorporate UMI.

The materials are as described in Example 1.

The method comprises tagmenting target DNA with a 5′ sequencing adapter (a 5′ adapter), then hybridizing a 3′ sequencing adapter (a 3′ adapter) to the 5′ adapter ME sequence such that a UMI is placed directly adjacent to the 3′ end of the insert DNA. This produces an in-line UMI, which ensures compatibility with standard, downstream library preparation steps (i.e., sample multiplexing PCR) and sequencing chemistry recipes.

Tagmentation is performed on double-stranded DNA with a transposome containing only the 5′ adapter sequence, A14, and the non-transferred Tn5-mosaic-end sequence, ME, is denatured. The 3′ adapter is an oligonucleotide that contains a 3′ universal hybridizing tail, which may comprise inosine bases capable of universal Watson-Crick base pairing. The 3′ universal hybridizing tail further contains a UMI hairpin, and ME′ sequence, and the 3′ adapter sequence, B15.

The 3′ adapter is hybridized to the 5′ adapter ME using Hyb2Y. The universal hybridizing tail is hybridized to the exposed 5′ bases of the transferred strand (adjoined to the 5′ adapter). Using a 9-nucleotide universal hybridizing tail, the exposed 9 nucleotides of the transferred strand hybridize completely, and the 5′ of the universal hybridizing tail is ligated to the 3′ of the non-transferred strand by E. coli DNA ligase. Using a universal hybridizing tail of less than 9 nucleotides may require an additional extension step of the non-transferred strand prior to ligation.

Using a standard sequencing method (as described in Example 2 and shown in FIGS. 3B and 20), the library of this example may be sequenced at the beginning of read 2 or at the end of read 1, preceding and proceeding the insert DNA, respectively. The read is more likely to be captured at the beginning of read 2 due to the quality of inserts and variable insert lengths.

The universal hybridizing tail oligonucleotide provides the potential to track and resolve the unique copies of each (original) DNA molecule (unique copy index, UCI). Different copies of an original insert molecule can have different 9 nucleotide universal hybridizing tail sequences by the same UMI. Like the UMI, the UCI is in-line, with pre-defined positions in the sequencing read. Thus, it can be identified bioinformatically.

### Example 14. Preparation of a DNA Library for Sequencing Using a 3′ Adapter Comprising a Hairpin-UMI

This example describes an asymmetrical tagmentation BLT method used to prepare a DNA sequencing library with in-line UMIs wherein the UMI is incorporated after tagmentation (FIG. 15). A 3′ adapter comprising a hairpin-UMI is used to incorporate UMI.

The materials are as described in Example 1.

The 3′ adapter contains a hairpin UMI as described in Example 13, but it does not contain a universal hybridizing tail.

The 5′ adapter tagmentation and 3′ adapter hybridization steps are performed as described in Example 13. After 3′ adapter hybridization, the 3′ of the non-transferred strand is extended by a DNA polymerase until it reaches the 5′ end of the hybridized 3′ adapter. (The DNA polymerase contains no strand displacement and no 5′ to 3′ exonuclease activity.) this places the 5′ end of the UMI-hairpin in close proximity to the 3′ end of the 3′ adapter.

Using a standard sequencing method (as described in Example 2 and shown in FIGS. 3B and 20), the library of this example may be sequenced at the beginning of read 2 or at the end of read 1, preceding and proceeding the insert DNA, respectively. The read is more likely to be captured at the beginning of read 2 due to the quality of inserts and variable insert lengths.

### Example 15a. Preparation of a DNA Library for Sequencing Using a 3′ Splint Ligation Adapter

This example describes an asymmetrical tagmentation BLT method used to prepare a DNA sequencing library with in-line UMIs wherein the UMI is incorporated after tagmentation (FIG. 16). A 3′ splint ligation adapter is used to incorporate UMI.

The materials are as described in Example 1.

The 5′ adapter tagmentation and 3′ adapter hybridization steps are performed as described in Example 13.

The 3′ splint ligation adapter is a partially double-stranded complex that creates a splint for ligation between UMI-ME′-B15 and the non-transferred strand (FIG. 16). Each strand of the 3′ splint ligation adapter forms one of two portions of the adapter, and each strand is about 50 nucleotides long. The two portions of the adapter are the splint (see FIG. 16, 3′ splint ligation adapter, bottom strand), and the tail (see FIG. 16, 3′ splint ligation adapter, top strand). The adapter splint portion contains the following regions from 5′ to 3′: ME, UMI′, ME′, truncated A14′. Both the ME and A14′ sequences may be truncated to improve desired hybridization specificity and to decrease adapter oligonucleotide costs. For example, ME is truncated to prevent intramolecular hybridization with the full ME′ sequence required for 5′ to 3′ adapter binding. The adapter tail portion hybridizes to the adapter splint portion through the UMI and ME sequences, which may improve efficiency by stabilizing hybridization between the 5′ adapter and the 3′ adapter. The adapter tail portion contains the following regions from 5′ to 3′: UMI, ME′, and B15. The adapter tail portion is not truncated. The non-transferred strand of the target DNA is extended to the 5′ end of the tail of the adapter and is ligated as specified according to the ligation step described in Example 14.

Using a standard sequencing method (as described in Example 2 and shown in FIGS. 3B and 20), the library of this example may be sequenced at the beginning of read 2 or at the end of read 1, preceding and proceeding the insert DNA, respectively.

### Example 15b. Preparation of a DNA Library for Sequencing Using a 3′ Splint Ligation Adapter

This example describes an asymmetrical tagmentation BLT method used to prepare a DNA sequencing library with in-line UMIs wherein the UMI is incorporated after tagmentation (FIG. 16). A 3′ splint ligation adapter is used to incorporate UMI. This example describes a method as provided by Example 15a with the following modifications.

The 3′ splint ligation adapter is as described in Example 15a above with the following modifications. The adapter splint portion contains the following regions from 5′ to 3′: X, UMI′, ME′. Compared to the splint portion of Example 15a, the splint portion in this example does not contain A14′ so that the 3′ splint adapter can facilitate on-bead 3′ adapter addition. The X sequence is a part of the 3′ TruSeq™ adapter sequence may be truncated to improve desired hybridization specificity and to decrease adapter oligonucleotide costs. The adapter tail portion contains the following regions from 5′ to 3′: UMI, X′ and B15.

The library of this example is sequenced using a standard sequencing method (as described in Example 2 and shown in FIGS. 3B and 20) with the following modification—a custom read 2 primer is needed.

### Example 16a. Preparation of a DNA Library for Sequencing Using a 3′ Template Switch Oligonucleotide

This example describes an asymmetrical tagmentation BLT method used to prepare a DNA sequencing library with in-line UMIs wherein the UMI is incorporated after tagmentation (FIG. 17). A 3′ template switch oligonucleotide is used to incorporate UMI.

The materials are as described in Example 1.

The 3′ template switch oligonucleotide is about 70 nucleotides long and contains the following regions from 5′ to 3′: B15′, ME or X, UMI′, ME′, and A14′.

The 5′ adapter tagmentation and 3′ adapter hybridization steps are performed as described in Example 13. After hybridization, extension is performed with a polymerase capable of DNA-directed template switching, such as the murine leukemia virus (MMLV) reverse transcriptase. The non-transferred strand is extended to copy the 5′ end of the transferred strand by 9 nucleotides. Upon reaching the template switch junction (** in FIG. 17), the polymerase can switch from using the non-transferred DNA strand as a template, to the 3′ template switch oligonucleotide. In this way, the UMI, ME′/X′, and B15 sequences are copied from the 3′ template switch oligonucleotide.

Using a standard sequencing method (as described in Example 2 and shown in FIGS. 3B and 20), the library of this example may be sequenced at the beginning of read 2 or at the end of read 1, preceding and proceeding the insert DNA, respectively.

### Example 16b. Preparation of a DNA Library for Sequencing Using a 3′ Template Switch Oligonucleotide

This example describes an asymmetrical tagmentation BLT method used to prepare a DNA sequencing library with in-line UMIs wherein the UMI is incorporated after tagmentation (FIG. 17). A 3′ template switch oligonucleotide is used to incorporate UMI. This example describes a method as provided by Example 16a with the following modification in the 3′ template switch oligonucleotide.

The A14′ sequence of 3′ template switch oligonucleotide is either truncated or eliminated to facilitate on-bead addition of the 3′ template switch oligonucleotide.

Using a standard sequencing method (as described in Example 2 and shown in FIGS. 3B and 20), the library of this example may be sequenced at the beginning of read 2 or at the end of read 1, preceding and proceeding the insert DNA, respectively.

### Example 16c. Preparation of a DNA Library for Sequencing Using a 5′ Single-Stranded Polymerase Template Switch Oligonucleotide

This example describes an asymmetrical tagmentation BLT method used to prepare a DNA sequencing library with in-line UMIs wherein the UMI is incorporated after tagmentation (FIGS. 18A-D). A 5′ polymerase template switch oligonucleotide is used to incorporate UMI.

The materials are as described in Example 1. Circulating tumor DNA (ctDNA) is used as the target DNA.

The 5′ single-stranded polymerase template switch oligonucleotide is a 5′ adapter with the following regions from 5′ to 3′: B15, X, and UMI (FIG. 18B).

The tagmentation and adapter hybridization steps are performed as described in Example 13 (FIGS. 18A-B). In this example, the 5′ adapter is appended to the 5‘ of ME’ (FIG. 18B).

Then, a polymerase template switch is used to add the 5′ adapter to the DNA insert. The polymerase switches from using the insert DNA as a template to using the appended 5′ adapter as a template (FIG. 18C). Upon completion of extending, the B15, X, and UMI sequences are fused to the 3′ end of the insert DNA and can be used as a template in PCR reaction to add additional flowcell and sample index adapter elements (FIG. 18D).

The library of this example is sequenced using a standard sequencing method (as described in Example 2). The X region serves to extend the B15 region so that a suitable Tm is reached for sequencing from B15 in the absence of ME.

### Example 16d. Preparation of a DNA Library for Sequencing Using a 5′ Double-Stranded Adapter, Polymerase Extension and Proximity Ligation

This example describes an asymmetrical tagmentation BLT method used to prepare a DNA sequencing library with in-line UMIs wherein the UMI is incorporated after tagmentation (FIGS. 19A-D). A 5′ double-stranded adapter is used to incorporate UMI.

The materials are as described in Example 1. Circulating tumor DNA (ctDNA) is used as the target DNA.

In this example, the 5′ double-stranded adapter contains the following regions on its first strand from 5′ to 3′: B15, X, and UMI. The second strand contains the complementary sequences, listed here from 5′ to 3′: UMI′, X′, and B15′. While a 5′-phosphate is present on the second strand of the 5′ adapter, the ME′ on the tagmentation adapter is dephosphorylated to prevent ligation of the ME′ with the 5′ adapter (FIG. 19B).

The tagmentation and adapter hybridization steps are performed as described in Example 13 (FIGS. 19A-B). The 5′ adapter is appended to the 5‘ of ME’ (FIG. 19B). During adapter hybridization, the first and second strands of the 5′ adapter are mixed to form a double strand. Also, the ME′ on the tagmentation adapter is dephosphorylated to prevent ligation with the 5′ adapter (FIG. 19B).

Then, a polymerase, such as a T4 DNA pol Exo- (New England BioLabs, Catalog #M0203S) or Ttaq608, is used to extend across the gap from the initial transposition reaction (FIG. 19C). Taq polymerase, or mutants, analogues, or derivatives of any of the aforementioned polymerases may also be used in this step instead. The polymerase used is lacking in strand displacement or exonuclease activity. Gap extension terminates at the junction with ME′.

Then, a proximity ligation step occurs between the 3′ extension product and the second strand of the 5′ adapter (FIG. 19C).

The library of this example (FIG. 19D) is sequenced using a standard sequencing method (as described in Example 2). The X region serves to extend the B15 region so that a suitable Tm is reached for sequencing from B15 in the absence of ME. The read is more likely to be captured at the beginning of read 2 due to the quality of inserts and variable insert lengths.

### Example 17. Preparation of DNA Libraries for the Detection of Low Frequency Variants

This example describes an asymmetrical tagmentation BLT method used to prepare a DNA sequencing library for the detection of low frequency single nucleotide variants (SNVs) and structural variants (SVs).

A first DNA library is prepared using the method described in Example 7 above. A second DNA library is prepared using the TruSeq™ method.

DNA is used containing SNVs and SVs at specific amounts, i.e., 2%, 0.5% and 0.2%.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method of producing a double-stranded nucleic acid library wherein each fragment in the library comprises a unique molecular identifier (UMI) wherein the method comprises:
a. applying a sample comprising double-stranded target nucleic acids to a first transposome complex comprising:
i. a first transposase,
ii. a first transposon comprising a first 3′ end transposon end sequence, a first adapter sequence, and a first UMI, and
iii. a second transposon comprising a sequence all or partially complementary to the first 3′ end transposon end sequence;
b. tagmenting the double-stranded target nucleic acids with the first transposome complex to produce tagmented double-stranded target nucleic acid fragments, wherein each tagmented double-stranded target nucleic acid fragment comprises the first adapter sequence and the first UMI,
c. releasing the tagmented double-stranded target nucleic acid fragments from the first transposome complex,
d. optionally extending the tagmented double-stranded target nucleic acid fragments,
e. optionally ligating the first transposon with the tagmented double-stranded target nucleic acid fragments or with the extended, tagmented double-stranded target nucleic acid fragments,
f. producing tagmented double-stranded target nucleic acid fragments, and
g. amplifying the tagmented double-stranded target nucleic acid fragments.

2. The method of claim 1, wherein the first UMI in the first transposon is located between the first adapter sequence and the first 3′ transposon end sequence.

3. The method of claim 1 or 2, wherein the first adapter sequence in the first transposon is located between the first UMI and the first 3′ transposon end sequence.

4. The method of any one of claims 1-3, further comprising a second transposome complex comprising:
a. a second transposase,
b. a third transposon comprising a second adapter sequence and a second 3′ transposon end sequence, and
c. a fourth transposon comprising a sequence all or partially complementary to the second 3′ end transposon end sequence.

5. The method of claim 4, wherein the tagmenting step produces tagmented double-stranded target nucleic acid fragments comprising:
a. a first strand comprising the first adapter sequence and the first UMI, and
b. a second strand comprising the second adapter sequence.

6. The method of claim 4 or 5, wherein
a. the third transposon further comprises a second UMI, and
b. the second adapter sequence is located between the second UMI and the second 3′ transposon end sequence.

7. The method of claim 6, wherein the tagmenting step produces double-stranded target nucleic acid fragments comprising:
a. a first strand comprising the first adapter sequence and the first UMI, and
b. a second strand comprising the second adapter sequence and the second UMI.

8. A method of producing a double-stranded nucleic acid library wherein each fragment in the library comprises a UMI wherein the method comprises:
a. applying a sample comprising double-stranded target nucleic acids to a transposome complex comprising:
i. a transposase,
ii. a first transposon comprising a first 3′ end transposon end sequence and a first adapter sequence, and
iii. a second transposon comprising a sequence all or partially complementary to the first 3′ end transposon end sequence;
b. tagmenting a first strand of the double-stranded target nucleic acids with the transposome complex to produce tagmented double-stranded target nucleic acid fragments, wherein each tagmented double-stranded target nucleic acid fragment comprises the first adapter sequence,
c. releasing the tagmented double-stranded target nucleic acid fragments from the transposome complex,
d. hybridizing a polynucleotide comprising a second adapter sequence, a UMI, and a sequence all or partially complementary to the first 3′ end transposon sequence,
e. optionally extending a second strand of the tagmented double-stranded target nucleic acid fragments,
f. optionally ligating the polynucleotide with the tagmented double-stranded target nucleic acid fragments or with the extended tagmented double-stranded target nucleic acid fragments,
g. producing tagmented double-stranded target nucleic acid fragments comprising the UMI, wherein the UMI is located directly adjacent to the 3′ end of an insert DNA, and
h. amplifying the tagmented double-stranded target nucleic acid fragments comprising the UMI.

9. A method of producing a double-stranded nucleic acid library wherein each fragment in the library comprises a UMI wherein the method comprises:
a. applying a sample comprising double-stranded target nucleic acids to a transposome complex comprising:
i. a transposase,
ii. a first transposon comprising a first 3′ end transposon end sequence and a first adapter sequence, and
iii. a second transposon comprising a sequence all or partially complementary to the first 3′ end transposon end sequence;
b. tagmenting a first strand of the double-stranded target nucleic acids with the transposome complex to produce tagmented double-stranded target nucleic acid fragments, wherein each tagmented double-stranded target nucleic acid fragment comprises the first adapter sequence,
c. releasing the tagmented double stranded target nucleic acid fragments from transposome complex,
d. hybridizing a first polynucleotide comprising a UMI, and a second adapter sequence,
e. optionally adding a second polynucleotide comprising regions complementary to the first polynucleotide to produce a double-stranded adapter,
f. optionally extending a second strand of the tagmented double-stranded target nucleic acid fragments,
g. optionally ligating the second polynucleotide with the second strand of the extended tagmented double-stranded target nucleic acid fragments,
h. producing tagmented double stranded target nucleic acid fragments comprising the UMI, wherein the UMI is located between the double-stranded target nucleic acid fragments and the second adapter sequence, and
i. amplifying the tagmented double-stranded target nucleic acid fragments comprising the UMI.

10. The method of claim 9, wherein after the hybridizing step, the method further comprises
a. extending a second strand of the double-stranded target nucleic acid fragments, and
b. copying the first polynucleotide.

11. A method of producing a double-stranded nucleic acid library wherein each fragment in the library comprises two different UMIs wherein the method comprises
a. applying a sample comprising double-stranded target nucleic acids to:
i. a first transposome complex comprising:
1. a first transposase and
2. a first forked adapter comprising (a) a first transposon on a first strand of the double-stranded target nucleic acid fragments, and (b) a second transposon, wherein
the first transposon comprises a first 3′ end transposon end sequence, a first copy of a first adapter sequence, and a first UMI, and
the second transposon comprises a first copy of a second adapter sequence, and a sequence all or partially complementary to the first 3′ end transposon end sequence and the first UMI;
further wherein the first copy of the first adapter sequence is single-stranded and the first copy of the second adapter sequence includes a double-stranded portion; and
ii. a second transposome complex comprising:
1. a second transposase and
2. a second forked adapter comprising (a) a third transposon on a second strand of the double-stranded target nucleic acid fragments, and (b) a fourth transposon, wherein
the third transposon comprises a second 3′ end transposon end sequence, a second copy of the first adapter sequence, and a second UMI, and
the third transposon comprises a second copy of the second adapter sequence, and a sequence all or partially complementary to the second 3′ end transposon end sequence and the second UMI;
further wherein the second copy of the first adapter sequence is single-stranded and the second copy of the second adapter sequence includes a double-stranded portion;
b. tagmenting the double-stranded target nucleic acids with the forked adapters to produce tagmented double-stranded target nucleic acid fragments, wherein each tagmented double-stranded target nucleic acid fragment comprises the first and second copies of the first adapter sequence, the first UMI, the first and second copies of the second adapter sequence, and the second UMI,
c. releasing the tagmented double-stranded target nucleic acid fragments from the transposome complexes,
d. optionally extending the tagmented double-stranded target nucleic acid fragments,
e. ligating the second and fourth transposons with the double-stranded target nucleic acid fragments or with the extended tagmented double-stranded target nucleic acid fragments,
f. producing tagmented double-stranded target nucleic acid fragments, and
g. amplifying the tagmented double-stranded target nucleic acid fragments.

12. A method of producing a double-stranded nucleic acid library wherein each fragment in the library comprises four different UMIs wherein the method comprises
a. applying a sample comprising double-stranded target nucleic acids to:
i. a first transposome complex comprising:
1. a first transposase and
2. a first forked adapter comprising (a) a first transposon on a first strand of the double-stranded target nucleic acid fragments, and (b) a second transposon, wherein
the first transposon comprises a first 3′ end transposon end sequence, a first copy of a first adapter sequence, a first copy of a first UMI, and a first copy of a second adapter sequence, and
the second transposon comprises a sequence all or partially complementary to the first 3′ end transposon end sequence, a first copy of a third adapter sequence, a first copy of a second UMI, and a fourth adapter sequence;
further wherein the first copies of the first, second, and third adapter sequences are single-stranded and the fourth adapter sequence includes a double-stranded portion; and
ii. a second transposome complex comprising:
1. a second transposase and
2. a second forked adapter comprising (a) a third transposon on a second strand of the double-stranded target nucleic acid fragments, and (b) a fourth transposon, wherein
the third transposon comprises a second 3′ end transposon end sequence, a first copy of a fifth adapter sequence, a first copy of a third UMI, and a first copy of a sixth adapter sequence;
the fourth transposon comprises a sequence all or partially complementary to the second 3′ end transposon end sequence, a first copy of a seventh adapter sequence, a first copy of a fourth UMI, and an eighth adapter sequence;
further wherein the first copies of the fifth, sixth, and seventh adapter sequences are single-stranded and the eighth adapter sequence includes a double-stranded portion;
b. tagmenting the double-stranded target nucleic acids with the forked adapters to produce tagmented double-stranded target nucleic acid fragments, wherein each tagmented double-stranded target nucleic acid fragment comprises the first copies of the first, second, third, fifth, sixth, and seventh adapter sequences; the first copies of the first, second, third, and fourth UMIs; the sixth adapter sequence; and the eighth adapter sequence,
c. releasing the tagmented double-stranded target nucleic acid fragments from the transposome complexes,
d. optionally extending the tagmented double-stranded target nucleic acid fragments,
e. ligating the second and fourth transposons with the double-stranded target nucleic acid fragments or with the extended tagmented double-stranded target nucleic acid fragments,
f. producing tagmented double-stranded target nucleic acid fragments, and
g. amplifying the tagmented double-stranded target nucleic acid fragments.

13. The method of any one of claims 6, 7, 11 or 12, wherein the first, second, third, and fourth UMIs may be complementary or different sequences.

14. The method of any one of claims 1-13, wherein the double-stranded target nucleic acids are double-stranded DNA.

15. The method of any one of claims 1-13, wherein the double-stranded target nucleic acids are ctDNA.

16. The method of any one of claims 1-13, wherein the double-stranded target nucleic acids are cfDNA.

17. The method of any one of claims 1-13, wherein the double-stranded target nucleic acids are RNA.

18. The method of any one of claims 1-13, wherein double-stranded target nucleic acids are cDNA or DNA:RNA duplexes are generated from RNA.

19. The method of any one of claims 1-18, wherein the first adapter sequence is a 5′ first-read sequencing adapter sequence.

20. The method of any one of claims 1-19, wherein the second adapter sequence is a 5′ second-read sequencing adapter sequence.

21. The method of any one of claims 1-20, wherein the first and second adapter sequences are 5′ first-read and 5′ second-read sequencing adapter sequences.

22. The method of any one of claims 1-21, wherein the 5′ first-read and 5′ second-read sequencing adapter sequences comprise unique primer binding sites.

23. The method of any one of claims 1, 2, 4-8, or 13-22, wherein the first UMI is on the first strand of the tagmented double-stranded target nucleic acid fragments.

24. The method of any one of claims 1, 3, 5-7, 13-22, wherein a first copy of the first UMI is on the first strand and a second copy of the first UMI is on the second strand of the tagmented double-stranded target nucleic acid fragments.

25. The method of any one of claims 1-7, 13-22, wherein the first UMI is on the first strand of the tagmented double-stranded target nucleic acid fragments, the second UMI is on the second strand of the tagmented double-stranded target nucleic acid fragments.

26. The method of any one of claims 1-25, wherein the first, second, third, or fourth transposon further comprises a biotin tag.

27. The method of any one of claims 1-26, wherein the first, second, third, or fourth transposon further comprises a first unique primer binding sequence.

28. The method of claim 27, wherein the first, second, third, or fourth transposon further comprises a second unique primer binding sequence.

29. The method of claim 27 or 28, wherein the unique primer binding sequence comprises A2, A14, and/or B15.

30. The method of any one of claims 8-10 or 14-22, wherein the hybridizing step generates a forked adapter.

31. The method of any one of claims 1-30, further comprising extending from a 3′ end of the double-stranded target nucleic acid fragments to a 5′ end of the transposons.

32. The method of any one of claims 1-7 or 11-31, wherein the ligating step comprises ligating a 3′ end of the tagmented double-stranded target nucleic acid fragments or a 3′ end of the extended tagmented double-stranded target nucleic acid fragments with a 5′ end of the first, second, or fourth transposon.

33. The method of any one of claims 1-32, wherein the extension and/or ligating step is optionally performed in an extension ligation mix.

34. The method of any one of claims 8, 15-22, 26-33, wherein the polynucleotide comprises a 3′ adapter comprising:
a. a hairpin UMI,
b. a hairpin UMI and a universal hybridizing tail,
c. a splint ligation adapter, or
d. a 3′ template switch oligonucleotide.

35. The method of claim 34, wherein the hairpin UMI is stable during the extending step and/or the ligating step, but not during the amplifying step.

36. The method of claim 34 or 35, wherein the hairpin UMI comprises a 3 or 4 base pair stem.

37. The method of any one of claims 34-36, wherein the universal hybridizing tail comprises nucleotides that can bind to any DNA nucleotide.

38. The method of any one of the claims 34-37, wherein the ligating step comprises ligating a 3′ end of the second strand of the tagmented double-stranded target nucleic acid fragments with a 5′ end of the universal hybridization tail.

39. The method of claim 34, wherein
a. the polynucleotide comprises a 3′ adapter comprising a hairpin UMI, and
b. the extending step comprises extending from a 3′ end of the second strand of the tagmented double-stranded target nucleic acid fragments to a 5′ end of the hairpin UMI.

40. The method of claim 39, wherein the ligating step comprises ligating the 3′ end of second strand of the extended tagmented double-stranded target nucleic acid fragments with the 5′ end of the hairpin UMI.

41. The method of claim 34, wherein
a. the polynucleotide comprises a splint ligation adapter, and
b. the extending step comprises extending from a 3′ end of the second strand of the tagmented double-stranded target nucleic acid fragments to a 5′ end of the splint ligation adapter.

42. The method of claim 41, wherein the extending step comprises extending 9 bases.

43. The method of claim 41 or 42, wherein the ligating step comprises ligating the 3′ end of the second strand of the extended tagmented double-stranded target nucleic acid fragments with a 5′ end of a first strand of the splint ligation adapter.

44. The method of any one of claim 34, wherein
a. the polynucleotide comprises a template switch oligonucleotide, and
b. the extending step comprises extending from a 3′ end of the second strand of the tagmented double-stranded target nucleic acid fragments to a junction in the template switch oligonucleotide by copying the first strand of the tagmented double-stranded target nucleic acid fragments,
c. switching templates from the first strand to an unpaired region of the 3′ template switch oligonucleotide, and
d. copying the unpaired region of the 3′ template switch oligonucleotide from the junction to a 5′ end of the unpaired region of the 3′ template switch oligonucleotide.

45. The method of claim 44, wherein the extending, switching, and copying are performed by a polymerase capable of DNA-directed template-switching.

46. The method of claim 44 or 45, wherein the polymerase capable of DNA-directed template-switching comprises MMLV reverse transcriptase.

47. The method of any one of the claims 1-33, wherein the ligating step comprises ligating a 3′ end of the tagmented double-stranded target nucleic acid fragments with a 5′ end of first, second, or fourth transposon.

48. The method of any one of claims 1-33 or 47, further comprising selecting for amplified nucleic acid fragments within a size range after the amplifying step.

49. The method of any one of claims 1-48, wherein the amplifying step comprises adding oligonucleotides to one or both ends of the tagmented double-stranded target nucleic acid fragments for attaching the library to a solid support.

50. The method of any one of claims 1-49, wherein the amplifying step comprises adding at least a first-read sequencing oligonucleotide and/or a second-read sequencing oligonucleotide.

51. The method of any one of claims 1-50, wherein the amplifying step comprises adding at least a P5 oligonucleotide and a P7 oligonucleotide.

52. The method of any one of claims 1-51, wherein the amplifying step comprises adding at least a plurality of i5 oligonucleotides and a plurality of i7 oligonucleotides.

53. The method of any one of claims 1-52 wherein the transposome complex, the first transposome complex and/or the second transposome complex are on a solid support.

54. The method of any one of claims 1-53, wherein the transposome complex, the first transposome complex and/or the second transposome complex are in solution.

55. A method of sequencing a double-stranded nucleic acid library produced by the method of any one of claims 1-54, wherein the UMIs are sequenced to provide increased sensitivity in DNA sequencing.

56. The method of claim 55, comprising binding sequencing primers having similar melting temperatures.

57. The method of claim 55 or 56, comprising binding sequencing primers comprising a sequence all or partially complementary to unique primer binding sequences.

58. The method of any one of claims 55-57, comprising sequencing primers with at least an A2 sequence.

59. The method of any one of claims 55-57, comprising sequencing primers with at least an A14 sequence and a B15 sequence.

60. The method of any one of claims 55-59, comprising sequencing primers with at least a bridged primer.

61. The method of any one of claims 55-60, further comprising dark cycles wherein data is not being recorded for a portion of the sequencing method.

62. The method of any one of claims 55-60, wherein the data not being recorded is sequence data associated with the 3′ transposon end sequence.

63. The method of any one of claims 55-60, wherein the method obviates the need for dark cycles.

64. The method of claim 1 or 9, wherein the extension step comprises a polymerase to copy the UMI or the first UMI to produce a duplex UMI.

65. A transposome complex comprising:
a. a transposase,
b. a first transposon comprising a 3′ transposon end sequence and a 5′ adapter sequence, and
c. a second transposon comprising a sequence all or partially complementary to the first 3′ end transposon end sequence.

66. The transposome complex of claim 65, wherein the 5′ adapter sequence of the first transposon comprises an A14 sequence (SEQ ID NO: 4), an A2 sequence (SEQ ID NO: 7), and/or a B15 sequence (SEQ ID NO: 5).

67. The transposome complex of claim 65 or 66, wherein the first transposon further comprises a UMI sequence.

68. The transposome complex of any one of claims 65-67 wherein the first or second transposon comprises A14-ME (SEQ ID NO: 1).

69. The transposome complex of any one of claims 65-67 wherein the first or second transposon comprises B15-ME (SEQ ID NO: 2).

70. The transposome complex of any one of claims 65-67 wherein the 3′ transposon end sequence of the first transposon comprises ME (SEQ ID NO: 6) or ME′ (SEQ ID NO: 3).

71. The transposome complex of any one of claims 65-67 wherein the 3′ transposon end sequence of the second transposon comprises ME (SEQ ID NO: 6) or ME′ (SEQ ID NO: 3).

72. The transposome complex of claim 67, wherein the second transposon further comprises a 3′ adapter sequence, wherein
the 3′ adapter sequence of the second transposon is either partially or completely complementary to the 5′ adapter sequence of the first transposon.

73. The transposome complex of claim 67, wherein the second transposon further comprises a 3′ adapter sequence, wherein
no portion of the 3′ adapter sequence of the second transposon is complementary to the 5′ adapter sequence of the first transposon.

74. The transposome complex of claim 72 or 73, wherein the 3′ adapter sequence of the second transposon comprises an A14 sequence (SEQ ID NO: 4), an A2 sequence (SEQ ID NO: 7), a B15 sequence (SEQ ID NO: 5), an X sequence, a Y′ sequence, an A sequence, and/or a B sequence.

75. The transposome complex of claim 72 or 74, wherein the second transposon further comprises a sequence that is complementary to the UMI sequence of the first transposon.

76. The transposome complex of claim 73 or 74, wherein the second transposon further comprises a UMI, wherein the UMI of the second transposon comprises a different sequence from the UMI of the first transposon.

77. The transposome complex of claim 75 or 76, further comprising an oligonucleotide complementary to the B15 sequence or A14 sequence.

78. The transposome complex of claim 76, further comprising:
a. an A adapter sequence adjacent to the A14 sequence,
b. a B adapter sequence adjacent to the B15 sequence,
c. a X adapter sequence adjacent to the ME sequence, and/or
d. a Y′ adapter sequence adjacent to the ME′ sequence.

79. The transposome complex of any one of claims 65-78, wherein the transposome complex is immobilized to a solid support via the first or second transposon.

80. The transposome complex of claim 77, wherein the transposome complex is immobilized to a solid support via the complementary oligonucleotide.

81. The transposome complex of claim 79 or 80, wherein the solid support is a bead.

82. A kit comprising the transposome complex of any one of claims 65-81.

83. A kit for generating the transposome complex of any one of claims 65-81.
